(19) United States
(12) Patent Application Publication
(10) Pub. No.: US 2019/0364862 A1
(43) Pub. Date: Dec. 5, 2019

(54) DMD REPORTER MODELS CONTAINING HUMANIZED DUCHENNE MUSCULAR DYSTROPHY MUTATIONS

(71) Applicant: The Board of Regents of the University of Texas System, Austin, TX (US)

(72) Inventors: Leonela AMOASII, Dallas, TX (US); Chengzu LONG, New York, NY (US); Rhonda BASSEL-DUBY, Dallas, TX (US); Eric OLSON, University Park, TX (US)

(73) Assignee: The Board of Regents of the University of Texas System, Austin, TX (US)

(21) Appl. No.: 16/467,445

(22) PCT Filed: Dec. 8, 2017
(86) PCT No.: PCT/US2017/065268

§ 371 Date: Jun. 6, 2019

Related U.S. Application Data
(60) Provisional application No. 62/431,699, filed on Dec. 8, 2016.

(51) Int. Cl. A01K 67/027; C12N 15/90; C12N 9/22; C12N 15/113; C07K 14/47

(52) U.S. Cl. CPC A01K 67/0278; C12N 15/907; C12N 9/22; C12N 15/113; C07K 14/4708; A01K 2207/15; C12N 2310/20; A01K 2217/072; A01K 2217/15; A01K 2227/105; A01K 2267/0393; A01K 2267/0306; A01K 2217/052

(57) ABSTRACT

CRISPR/Cas9-mediated genome editing holds clinical potential for treating genetic diseases, such as Duchenne muscular dystrophy (DMD), which is caused by mutations in the dystrophin gene. In vivo AAV-mediated delivery of gene-editing components machinery has been shown to successfully remove mutant sequence to generate an exon skipping in the cardiac and skeletal muscle cells of postnatal mdx mice, a model of DMD. Using different modes of AAV9 delivery, the restoration of dystrophin protein expression in cardiac and skeletal muscle of mdx mice was achieved. Here, a humanized mouse model for DMD is created to help test the efficacy of genome editing to cure DMD. Additionally, to facilitate the analysis of exon skipping strategies in vivo in a non-invasive way, a reporter luciferase knock-in version of the mouse model was prepared. These humanized mouse models provide the ability to study correcting of mutations responsible for DMD in vivo.

### PRIORITY CLAIM

The present application claims benefit of priority to U.S. Provisional Application Ser. No. 62/431,699, filed Dec. 8, 2016, the entire contents of which are hereby incorporated by reference.

### FEDERAL FUNDING SUPPORT CLAUSE

This invention was made with government support under grant no. U54 HD 087351 awarded by National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Dec. 7, 2017, is named UTFD_P3125WO.txt and is 186,485 bytes in size.

### FIELD OF THE DISCLOSURE

The present disclosure relates to the fields of molecular biology, medicine and genetics. More particularly, the disclosure relates to the use of genome editing to create humanized animal models for different forms of Duchenne muscular dystrophy (DMD), each containing distinct DMD mutations.

### BACKGROUND

Muscular dystrophies (MD) are a group of more than 30 genetic diseases characterized by progressive weakness and degeneration of the skeletal muscles that control movement. Duchenne muscular dystrophy (DMD) is one of the most severe forms of MD that affects approximately 1 in 5000 boys and is characterized by progressive muscle weakness and premature death. Cardiomyopathy and heart failure are common, incurable and lethal features of DMD. The disease is caused by mutations in the gene encoding dystrophin (DMD), a large intracellular protein that links the dystroglycan complex at the cell surface with the underlying cytoskeleton, thereby maintaining integrity of the muscle cell membrane during contraction. Mutations in the dystrophin gene result in loss of expression of dystrophin causing muscle membrane fragility and progressive muscle wasting.

### SUMMARY

Despite intense efforts to find cures through a variety of approaches, including myoblast transfer, viral delivery, and oligonucleotide-mediated exon skipping, there remains no cure for any type of muscular dystrophy. The present inventors recently used clustered regularly interspaced short palindromic repeat/Cas9 (CRISPR/Cas9)-mediated genome editing to correct the dystrophin gene (DMD) mutation in postnatal mdx mice, a model for DMD. In vivo AAV-mediated delivery of gene-editing components successfully removed the mutant genomic sequence by exon skipping in the cardiac and skeletal muscle cells of mdx mice. Using different modes of AAV9 delivery, the inventors restored dystrophin protein expression in cardiac and skeletal muscle of mdx mice. The mdx mouse model and the correction exon 23 using AAV delivery of myoediting machinery has been useful to show proof-of concept of exon skipping approach using several cuts in genomic region encompassing the mutation in vivo. However, there is a lack of other models for the various known DMD mutations, and for new mutations that continue to be discovered.

In some embodiments, a composition comprises a sequence encoding a Cas9 polypeptide, a sequence encoding a first guide RNA (gRNA) targeting a first genomic target sequence, and a sequence encoding a second gRNA targeting a second genomic target sequence, wherein the first and second genomic target sequences each comprise an intronic sequence surrounding an exon of the murine dystrophin gene. In some embodiments, the exon comprises exon 50 of the murine dystrophin gene. In some embodiments, the sequence encoding a Cas9 polypeptide is isolated or derived from a sequence encoding a S. aureus Cas9 polypeptide. In some embodiments, at least one of the sequence encoding the Cas9 polypeptide, the sequence encoding the first gRNA, or the sequence encoding the second gRNA comprises an RNA sequence. In some embodiments, the RNA sequence comprises an mRNA sequence. In some embodiments, the RNA sequence comprises at least one chemically-modified nucleotide. In some embodiments, at least one of the sequence encoding the Cas9 polypeptide, the sequence encoding the first gRNA, or the sequence encoding the second gRNA comprises a DNA sequence.

In some embodiments, a first vector comprises the sequence encoding the Cas9 polypeptide and a second vector comprises at least one of the sequence encoding the first gRNA or the sequence encoding the second gRNA. In some embodiments, the first vector or the sequence encoding the Cas9 polypeptide further comprises a first polyA sequence. In some embodiments, the second vector or the sequence encoding the first gRNA or the sequence encoding the second gRNA encodes a second polyA sequence. In some embodiments, the first vector or the sequence encoding the Cas9 polypeptide further comprises a first promoter sequence. In some embodiments, the second gRNA comprises a second promoter sequence.

In some embodiments, the first promoter sequence and the second promoter sequence are identical. In some embodiments, the first promoter sequence and the second promoter sequence are not identical. In some embodiments, the first promoter sequence or the second promoter sequence comprises a CK8 promoter sequence. In some embodiments, the first promoter sequence or the second promoter sequence comprises a CK8e promoter sequence. In some embodiments, the first promoter sequence or the second promoter sequence comprises a constitutive promoter. In some embodiments, the first promoter sequence or the second promoter sequences comprises an inducible promoter.

In some embodiments, at least one of the first vector and the second vector is a non-viral vector. In some embodiments, the non-viral vector is a plasmid. In some embodiments, a liposome or nanoparticle comprises the non-viral vector. In some embodiments, at least one of the first vector and the second vector is a viral vector. In some embodiments, the viral vector is an adeno-associated viral (AAV) vector. The AAV vector may be replication-defective or conditionally replication defective. In some embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

In some embodiments, one vector comprises the sequence encoding the Cas9 polypeptide, the sequence encoding the first gRNA and the sequence encoding the second gRNA. In embodiments, the vector further comprises a polyA sequence. In embodiments, the vector further comprises a promoter sequence. In embodiments, the promoter sequence comprises a constitutive promoter. In embodiments, the promoter sequence comprises an inducible promoter. In embodiments, the promoter sequence comprises a CK8 promoter sequence. In embodiments, the promoter sequence comprises a CK8e promoter sequence.

In embodiments, the composition comprises a sequence codon optimized for expression in a mammalian cell. In embodiments, the composition comprises a sequence codon optimized for expression in a human cell or a mouse cell. In some embodiments, the sequence encoding the Cas9 polypeptide is codon optimized for expression in human cells or mouse cells. In some embodiments, a composition of the disclosure further comprises a pharmaceutically carrier.

In some embodiments, a cell comprises a composition of the disclosure. In embodiments, the cell is a murine cell. In some embodiments, the cell is an oocyte. In embodiments, a composition may comprise the cell. In embodiments, a genetically engineered mouse may comprise the cell. In some embodiments, a method for creating a genetically engineered mouse comprises contacting the cell with a mouse.

In some embodiments, a genetically engineered mouse is provided, wherein the genome of the mouse comprises a deletion of exon 50 of the dystrophin gene resulting in an out of frame shift and a premature stop codon in exon 51 of the dystrophin gene. In some embodiments, the genetically engineered mouse further comprises a reporter gene located downstream of and in frame with exon 79 of the dystrophin gene, and upstream of a dystrophin 3′-UTR, wherein the reporter gene is expressed when exon 79 is translated in frame with exon 49. In some embodiments, the reporter gene is luciferase. In some embodiments, the genetically engineered mouse further comprises a protease coding sequence upstream of and in frame with the reporter gene, and downstream of and in frame with exon 79. In some embodiments, the protease is autocatalytic. In some embodiments, the protease is 2A protease.

In some embodiments, the genetically engineered mouse is heterozygous for a deletion. In some embodiments, the genetically engineered mouse is homozygous for a deletion. In some embodiments, the mouse exhibits increased creatine kinase levels compared to a wildtype mouse. In some embodiments, the mouse does not exhibit detectable dystrophin protein in heart or skeletal muscle.

In some embodiments, a method of producing a genetically engineered mouse comprises contacting a fertilized oocyte with CRISPR/Cas9 elements and two single guide RNA (sgRNA) targeting sequences flanking exon 50 of the dystrophin gene, thereby creating a modified oocyte, wherein deletion of exon 50 by CRISPR/Cas9 results in an out of frame shift and a premature stop codon in exon 51 of the dystrophin gene; and transferring the modified oocyte into a recipient female. In some embodiments, the oocyte comprises a dystrophin gene having a reporter gene located downstream of and in frame with exon 79 of the dystrophin gene, and upstream of a dystrophin 3′-UTR, wherein the reporter gene is expressed when exon 79 is translated in frame with exon 49. In some embodiments, the reporter gene is luciferase. In some embodiments, the oocyte comprises a protease coding sequence upstream of and in frame with the reporter gene, and downstream of and in frame with exon 79. In embodiments, the protease is autocatalytic. In embodiments, the protease is 2A protease. In embodiments, the mouse is heterozygous for a deletion. In embodiments, the mouse is homozygous for a deletion. In embodiments, wherein the mouse exhibits increased creatine kinase levels compared to a wildtype mouse. In embodiments, the mouse does not exhibit detectable dystrophin protein in heart or skeletal muscle.

In some embodiments, an isolated cell is obtained from a genetically engineered mouse of the disclosure. In some embodiments, the cell comprises a reporter gene located downstream of and in frame with exon 79 of the dystrophin gene, and upstream of a dystrophin 3′-UTR, wherein the reporter gene is expressed when exon 79 is translated in frame with exon 49. In some embodiments, the reporter gene is luciferase. In some embodiments, the cell comprises a protease coding sequence upstream of and in frame with the reporter gene, and downstream of and in frame with exon 79. In some embodiments, the protease is autocatalytic. In some embodiments, the protease is 2A protease. In some embodiments, the cell is heterozygous for a deletion. In some embodiments, the cell is homozygous for a deletion.

In some embodiments, a genetically engineered mouse is produced by a method comprising the steps of contacting a fertilized oocyte with CRISPR/Cas9 elements and two single guide RNA (sgRNA) targeting sequences flanking exon 50 of the dystrophin gene, thereby creating a modified oocyte, wherein deletion of exon 50 by CRISPR/Cas9 results in an out of frame shift and a premature stop codon in exon 51 of the dystrophin gene; and transferring the modified oocyte into a recipient female.

In some embodiments, a method of screening a candidate substance for DMD exon-skipping activity comprises contacting a mouse according to any of claim 43, 46, 47, or 74 with the candidate substance; and assessing in frame transcription and/or translation of exon 79 of the dystrophin gene, wherein the presence of in frame transcription and/or translation of exon 79 indicates the candidate substance exhibits exon-skipping activity.

In some embodiments, a method of producing a genetically engineered mouse comprises contacting a fertilized oocyte with CRISPR/Cpf1 elements and two single guide RNA (sgRNA) targeting sequences flanking exon 50 of the dystrophin gene, thereby creating a modified oocyte, wherein deletion of exon 50 by CRISPR/Cpf1 results in an out of frame shift and a premature stop codon in exon 51 of the dystrophin gene; and transferring the modified oocyte into a recipient female.

In some embodiments, a genetically engineered mouse is produced by a method comprising the steps of contacting a fertilized oocyte with CRISPR/Cpf1 elements and two single guide RNA (sgRNA) targeting sequences flanking exon 50 of the dystrophin gene, thereby creating a modified oocyte, wherein deletion of exon 50 by CRISPR/Cpf1 results in an out of frame shift and a premature stop codon in exon 51 of the dystrophin gene; and transferring the modified oocyte into a recipient female.

It is contemplated that any method or composition described herein can be implemented with respect to any other method or composition described herein.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-E. “Humanized”-ΔEx50 mouse model. (FIG. 1A) Outline of the CRISPR/Cas9 strategy used for generation of the mice. (FIG. 1B) RT-PCR analysis to validate the depletion of exon 50. (FIG. 1C) Sequence analysis of RT-PCR band to validate the depletion of exon and generation of an out of frame sequence (Nucleic Acid=tataaggaaa aaccaagcac tcagccagtg aagctgccag tcagactgtt actctagtga cac, SEQ ID NO: 805; Amino Acid=YKEKPSTQPVKLPVRL; SEQ ID NO: 806). (FIG. 1D) Serum creatine kinase (CK), a marker of muscle dystrophy that reflects muscle damage and membrane leakage was measured in wild type (WT), ΔEx50 and mdx mice. (FIG. 1E) Hematoxylin and eosin (H&E) and dystrophin staining of skeletal and cardiac muscle. Scale bar: 50 μm.

FIGS. 2A-B. Luciferase reporter mouse model. (FIG. 2A) Schematic of strategy for creation of dystrophin reporter mice. Dystrophin (Dmd) gene with exons is indicated in blue. Using CRISPR/Cas9 mutagenesis, the inventors inserted a Luciferase reporter with the protease 2A cleavage site at the 3′ end of the dystrophin coding region. (FIG. 2B) Bioluminescence imaging of wild-type (WT) and Dmd knock-in luciferase reporter mice.

FIGS. 3A-D. Luciferase Dmd-mutant reporter mouse model. (FIG. 3A) Schematic outline of strategy for generating Δex50-luciferase reporter mice. (FIG. 3B) Genotyping results of ΔEx50-Dmd-KI-luciferase reporter mice. Schematic view of genotyping strategy forward (Fw) and reverse (Rv) primers. (FIG. 3C) Bioluminescence imaging of wild-type (WT), Dmd knock-in luciferase reporter and Δex50-Dmd knock-in luciferase reporter mice. (FIG. 3D) Western blot analysis of dystrophin (DMD), Luciferin and vinculin (VCL) expression in skeletal muscle and heart tissues.

FIGS. 4A-D. Strategy for CRISPR/Cas9-mediated genome editing in ΔEx50-KI-luciferase mice. (FIG. 4A) Scheme showing the CRISPR/Cas9-mediated genome editing approach to correct the reading frame in ΔEx50-KI-luciferase mice by skipping exon 51. Gray exons are out of frame. (FIG. 4B) Illustration of sgRNA binding position and sequence for sgRNA-ex51-SA. PAM sequence for sgRNA is indicated in red. Black arrow indicates the cleavage site. (FIG. 4C) Genomic deep sequencing analysis of PCR amplicons generated across the exon 51 target site in 10T1/2 cells. Sequence of representative indels aligned with sgRNA sequence (indicated in blue) revealing insertions (highlighted in green) and deletions (highlighted in red). The line indicates the predicted exon splicing enhancers (ESEs) sequence located at the site of sgRNA. Black arrow indicates the cleavage site. (FIG. 4C) The muscle creatine kinase 8 (CK8e) promoter was used to express SpCas9. The U6, H1 and 7SK promoters for RNA polymerase III were used to express sgRNAs.

FIGS. 5A-D. In Vivo Investigation of Correction of dystrophin expression by intra-muscular injection of AAV9s. (FIG. 5A) TA muscles of ΔEx50-KI-luciferase mice were injected with AAV9s encoding sgRNA and Cas9. ΔEx50-KI-luciferase mice were analyzed weekly by bioluminescence. (FIG. 5B) Bioluminescence imaging of wild-type (WT), Dmd KI-luciferase reporter and ΔEx50-KI-luciferase reporter mice injected with AAV9s encoding sgRNA and Cas9 1 week and 3 weeks after injection. (FIG. 5C) Dystrophin immunohistochemistry of entire tibialis anterior muscle of wild-type (WT), Dmd KI-luciferase reporter and ΔEx50-KI-luciferase reporter mice injected with AAV9s encoding sgRNA and Cas9. (FIG. 5D) Dystrophin immunohistochemistry of tibialis anterior muscle of wild-type (WT), Dmd KI-luciferase reporter and ΔEx50-KI-luciferase reporter mice injected with AAV9s encoding sgRNA and Cas9.

### DETAILED DESCRIPTION

DMD is a new mutation syndrome with more than 4,000 independent mutations that have been identified in humans (world-wide web at dmd.nl). The majority of patient's mutations carry deletions that cluster in a hotspot, and thus a therapeutic approach for skipping certain exon applies to large group of patients. The rationale of the exon skipping approach is based on the genetic difference between DMD and Becker muscular dystrophy (BMD) patients. In DMD patients, the reading frame of dystrophin mRNA is disrupted resulting in prematurely truncated, non-functional dystrophin proteins. BMD patients have mutations in the DMD gene that maintain the reading frame allowing the production of internally deleted, but partially functional dystrophins leading to much milder disease symptoms compared to DMD patients.

One the most common hot spots in DMD is the between exons 45 and 51, where skipping of exon 51 would apply to the largest group (i.e., 13-14% of DMD mutations). To further assess the efficiency and optimize CRISPR/Cas9-mediated exon skipping in vivo, a mimic of the human “hot spot” region was generated in a mouse model by deleting exon 50 using CRISPR/Cas9 system directed by two single guide RNAs (sgRNAs). The ΔEx50 mouse model exhibits dystrophic myofibers and increased serum creatine kinase level, thus providing a representative model of DMD. To accelerate the analysis of exon skipping strategies in vivo and in a non-invasive way, a reporter mouse was generated by insertion of a Luciferase expression cassette into the 3′ end of the Dmd gene so that Luciferase would be translated in-frame with exon 79 of dystrophin. Then, the same 2 sgRNA were used to delete exon 50 in the Dmd-Luciferase line, generating a ΔEx50-Dmd-Luciferase mouse. Deletion of exon 50 in the Dmd-Luciferase line resulted in the decrease of bioluminescence signal in skeletal muscle and heart. These and other aspects of the disclosure are reproduced below.

### I. DUCHENNE MUSCULAR DYSTROPHY

A. Background Duchenne muscular dystrophy (DMD) is a recessive X-linked form of muscular dystrophy, affecting around 1 in 5000 boys, which results in muscle degeneration and premature death. The disorder is caused by a mutation in the gene dystrophin, (see GenBank Accession No. NC 000023.11), located on the human X chromosome, which codes for the protein dystrophin (GenBank Accession No. AAA53189; SEQ ID NO. 383), the sequence of which is reproduced below:

| | |
|---|---|
| 1 | mlwweevedc yeredvqkkt ftkwvnaqfs kfgkqhienl fsdlqdgrrl ldllegltgq |
| 61 | klpkekgstr vhalnnvnka lrvlqnnnvd lvnigstdiv dgnhkltlgl iwniilhwqv |
| 121 | knvmknimag lqqtnsekil lswvrqstrn ypqvnvinft tswsdglaln alihshrpdl |
| 181 | fdwnsvvcqq satqrlehaf niaryqlgie klldpedvdt typdkksilm yitslfqvlp |
| 241 | qqvsieaiqe vemlprppkv tkeehfqlhh qmhysqqitv slaqgyerts spkprfksya |
| 301 | ytqaayvtts dptrspfpsq hleapedksf gsslmesevn ldryqtalee vlswllsaed |
| 361 | tlqaqgeisn dvevvkdqfh thegymmdlt ahqgrvgnil qlgskligtg klsedeetev |
| 421 | qeqmnllnsr weclrvasme kqsnlhrvlm dlqnqklkel ndwltkteer trkmeeeplg |
| 481 | pdledlkrqv qqhkvlqedl eqeqvrvnsl thmvvvvdes sgdhataale eqlkvlgdrw |
| 541 | anicrwtedr wvllqdillk wqrlteeqcl fsawlseked avnkihttgf kdqnemlssl |
| 601 | qklavlkadl ekkkqsmgkl yslkqdllst lknksvtqkt eawldnfarc wdnlvqklek |
| 661 | staqisqavt ttqpsltqtt vmetvttvtt reqilvkhaq eelpppppqk krqitvdsei |
| 721 | rkrldvdite lhswitrsea vlqspefaif rkegnfsdlk ekvnaierek aekfrklqda |
| 781 | srsaqalveq mvnegvnads ikqaseqlns rwiefcqlls erlnwleyqn niiafynqlq |
| 841 | qleqmtttae nwlkiqpttp septaiksql kickdevnrl sglqpqierl kiqsialkek |
| 901 | gqgpmfldad fvaftnhfkq vfsdvqarek elqtifdtlp pmryqetmsa irtwvqqset |
| 961 | klsipqlsvt dyeimeqrlg elqalqsslq eqqsglyyls ttvkemskka pseisrkyqs |
| 1021 | efeeiegrwk klssqlvehc qkleeqmnkl rkiqnhiqtl kkwmaevdvf lkeewpalgd |
| 1081 | seilkkqlkq crllvsdiqt iqpslnsvne ggqkikneae pefasrlete lkelntqwdh |
| 1141 | mcqqvyarke alkgglektv slqkdlsemh ewmtqaeeey lerdfeyktp delqkaveem |
| 1201 | krakeeaqqk eakvklltes vnsviaqapp vaqealkkel eflttnyqwl ctrlngkckt |
| 1261 | leevwacwhe llsylekank wlnevefklk ttenipggae eisevldsle nlmrhsednp |
| 1321 | nqirilaqtl tdggvmdeli neeletfnsr wrelheeavr rqklleqsiq saqetekslh |
| 1381 | liqesltfid kqlaayiadk vdaaqmpqea qkiqsdltsh eisleemkkh nqgkeaaqry |
| 1441 | lsqidvaqkk lqdvsmkfrl fqkpanfelr lqeskmilde vkmhlpalet ksveqevvqs |
| 1501 | qlnhcvnlyk slsevkseve mviktgrqiv qkkqtenpke ldervtalkl hynelgakvt |
| 1561 | erkqqlekcl klsrkmrkem nvltewlaat dmeltkrsav egmpsnldse vawgkatqke |
| 1621 | iekqkvhlks itevgealkt vlgkketlve dklsllnsnw iavtsraeew lnllleyqkh |
| 1681 | metfdqnvdh itkwiiqadt lldesekkkp qqkedvlkrl kaelndirpk vdstrdqaan |
| 1741 | lmanrgdhcr klvepqisel nhrfaaishr iktgkasipl keleqfnsdi qkllepleae |
| 1801 | iqqgvnlkee dfnkdmnedn egtvkellqr gdnlqqritd erkreeikik qqllqtkhna |
| 1861 | lkdlrsqrrk kaleishqwy qykrqaddll kclddiekkl aslpeprder kikeidrelq |
| 1921 | kkkeelnavr rqaeglsedg aamaveptqi qlskrwreie skfaqfrrln faqihtvree |
| 1981 | tmmvmtedmp leisyvpsty lteithvsqa lleveqllna pdlcakdfed lfkqeeslkn |
| 2041 | ikdslqqssg ridiihskkt aalqsatpve rvklqealsq ldfqwekvnk mykdrqgrfd |
| 2101 | rsvekwrrfh ydikifnqwl teaeqflrkt qipenwehak ykwylkelqd gigqrqtyyr |
| 2161 | tlnatgeeii qqssktdasi lqeklgslnl rwqevckqls drkkrleeqk nilsefqrdl |
| 2221 | nefvlwleea dniasiplep gkeqqlkekl eqvkllveel plrqgilkql netggpvlvs |
| 2281 | apispeeqdk lenklkqtnl qwikvsralp ekqgeieaqi kdlgqlekkl edleeqlnhl |
| 2341 | llwlspirnq leiynqpnqe gpfdvqetei avqakqpdve eilskgqhly kekpatqpvk |
| 2401 | rkledlssew kavnrllqel rakqpdlapg lttigasptq tvtlytqpvv tketaiskle |
| 2461 | mpsslmlevp aladfnrawt eltdwlslld qviksqrvmv gdledinemi ikqkatmqdl |
| 2521 | eqrrpqleel itaaqnlknk tsnqeartii tdrieriqnq wdevqehlqn rrqqlnemlk |
| 2581 | dstqwleake eaeqvlgqar akleswkegp ytvdaiqkki tetkqlakdl rqwqtnvdva |
| 2641 | ndlalkllrd ysaddtrkvh miteninasw rsihkrvser eaaleethrl lqqfpldlek |
| 2701 | flawlteaet tanvlqdatr kerlledskg vkelmkqwqd lqgeieahtd vyhnldensq |
| 2761 | kilrslegsd davllqrrld nmnfkwselr kkslnirshl eassdqwkrl hlslqellvw |
| 2821 | lqlkddelsr qapiggdfpa vqkqndvhra fkrelktkep vimstletvr iflteqpleg |
| 2881 | leklyqepre lppeeraqnv trllrkqaee vnteweklnl hsadwqrkid etlerlqelq |
| 2941 | eatdeldlkl rqaevikgsw qpvgdllids lqdhlekvka lrgeiaplke nvshvndlar |
| 3001 | qlttlgiqls pynlstledl ntrwkllqva vedrvrqlhe ahrdfgpasq hflstsvqgp |
| 3061 | weraispnkv pyyinhetqt tcwdhpkmte lyqsladlnn vrfsayrtam klrrlqkalc |
| 3121 | ldllslsaac daldqhnlkq ndqpmdilqi inclttiydr leqehnnlvn vplcvdmcln |
| 3181 | wllnvydtgr tgrirvlsfk tgiislckah ledkyrylfk qvasstgfcd qrrlglllhd |
| 3241 | siqiprqlge vasfggsnie psvrscfqfa nnkpeieaal fldwmrlepq smvwlpvlhr |
| 3301 | vaaaetakhq akcnickecp iigfryrslk hfnydicqsc ffsgrvakgh kmhypmveyc |
| 3361 | tpttsgedvr dfakvlknkf rtkryfakhp rmgylpvqtv legdnmetpv tlinfwpvds |
| 3421 | apasspqlsh ddthsriehy asrlaemens ngsylndsis pnesiddehl liqhycqsln |
| 3481 | qdsplsqprs paqilisles eergeleril adleeenrnl qaeydrlkqq hehkglsplp |
| 3541 | sppemmptsp qsprdaelia eakllrqhkg rlearmqile dhnkqlesql hrlrqlleqp |
| 3601 | qaeakvngtt vsspstslqr sdssqpmllr vvgsqtsdsm geedllsppq dtstgleevm |
| 3661 | eqlnnsfpss rgrntpgkpm redtm |

In humans, dystrophin mRNA contains 79 exons. Dystrophin mRNA is known to be alternatively spliced, resulting in various isoforms. Exemplary dystrophin isoforms are listed in Table 1.

TABLE 1
Dystrophin isoforms

| | | Nucleic | | | |
|---|---|---|---|---|---|
| | | Acid | | Protein | |
| | | SEQ | | SEQ | |
| Sequence | Nucleic Acid | ID | Protein | ID | |
| Name | Accession No. | NO: | Accession No. | NO: | Description |
| DMD | NC_000023.11 | None | None | None | Sequence from |
| Genomic | (positions | | | | Human X |
| Sequence | 31119219 to | | | | Chromosome (at |
| | 33339609) | | | | positions Xp21.2 to |
| | | | | | p21.1) from |
| | | | | | Assembly |
| | | | | | GRCh38.p7 |
| | | | | | (GCF_000001405.33) |
| Dystrophin | NM_000109.3 | 384 | NP_000100.2 | 385 | Transcript Variant: |
| Dp427c | | | | | transcript Dp427c is |
| isoform | | | | | expressed |
| | | | | | predominantly in |
| | | | | | neurons of the cortex |
| | | | | | and the CA regions |
| | | | | | of the hippocampus. |
| | | | | | It uses a unique |
| | | | | | promoter/exon 1 |
| | | | | | located about 130 kb |
| | | | | | upstream of the |
| | | | | | Dp427m transcript |
| | | | | | promoter. The |
| | | | | | transcript includes |
| | | | | | the common exon 2 |
| | | | | | of transcript Dp427m |
| | | | | | and has a similar |
| | | | | | length of 14 kb. The |
| | | | | | Dp427c isoform |
| | | | | | contains a unique N- |
| | | | | | terminal MED |
| | | | | | sequence, instead of |
| | | | | | the |
| | | | | | MLWWEEVEDCY |
| | | | | | sequence of isoform |
| | | | | | Dp427m. The |
| | | | | | remainder of isoform |
| | | | | | Dp427c is identical |
| | | | | | to isoform Dp427m. |
| Dystrophin | NM_004006.2 | 386 | NP_003997.1 | 387 | Transcript Variant: |
| Dp427m | | | | | transcript Dp427m |
| isoform | | | | | encodes the main |
| | | | | | dystrophin protein |
| | | | | | found in muscle. As |
| | | | | | a result of alternative |
| | | | | | promoter use, exon 1 |
| | | | | | encodes a unique N- |
| | | | | | terminal |
| | | | | | MLWWEEVEDCY |
| | | | | | aa sequence. |
| Dystrophin | NM_004009.3 | 388 | NP_004000.1 | 389 | Transcript Variant: |
| Dp427p1 | | | | | transcript Dp427p1 |
| isoform | | | | | initiates from a |
| | | | | | unique |
| | | | | | promoter/exon 1 |
| | | | | | located in what |
| | | | | | corresponds to the |
| | | | | | first intron of |
| | | | | | transcript Dp427m. |
| | | | | | The transcript adds |
| | | | | | the common exon 2 |
| | | | | | of Dp427m and has a |
| | | | | | similar length (14 |
| | | | | | kb). The Dp427p1 |
| | | | | | isoform replaces the |
| | | | | | MLWWEEVEDCY- |
| | | | | | start of Dp427m with |
| | | | | | a unique N-terminal |
| | | | | | MSEVSSD aa |
| | | | | | sequence. |
| Dystrophin | NM_004011.3 | 390 | NP_004002.2 | 391 | Transcript Variant: |
| Dp260- | | | | | transcript Dp260-1 |
| 1 isoform | | | | | uses exons 30-79, |
| | | | | | and originates from a |
| | | | | | promoter/exon 1 |
| | | | | | sequence located in |
| | | | | | intron 29 of the |
| | | | | | dystrophin gene. As |
| | | | | | a result, Dp260-1 |
| | | | | | contains a 95 bp |
| | | | | | exon 1 encoding a |
| | | | | | unique N-terminal 16 |
| | | | | | aa |
| | | | | | MTEIILLIFFPAYFL |
| | | | | | N-sequence that |
| | | | | | replaces amino acids |
| | | | | | 1-1357 of the full- |
| | | | | | length dystrophin |
| | | | | | product (Dp427m |
| | | | | | isoform). |
| Dystrophin | NM_004012.3 | 392 | NP_004003.1 | 393 | Transcript Variant: |
| Dp260- | | | | | transcript Dp260-2 |
| 2 isoform | | | | | uses exons 30-79, |
| | | | | | starting from a |
| | | | | | promoter/exon 1 |
| | | | | | sequence located in |
| | | | | | intron 29 of the |
| | | | | | dystrophin gene that |
| | | | | | is alternatively |
| | | | | | spliced and lacks N- |
| | | | | | terminal amino acids |
| | | | | | 1-1357 of the full |
| | | | | | length dystrophin |
| | | | | | (Dp427m isoform). |
| | | | | | The Dp260-2 |
| | | | | | transcript encodes a |
| | | | | | unique N-terminal |
| | | | | | MSARKLRNLSYK |
| | | | | | K sequence. |
| Dystrophin | NM_004013.2 | 394 | NP_004004.1 | 395 | Transcript Variant: |
| Dp140 | | | | | Dp140 transcripts |
| isoform | | | | | use exons 45-79, |
| | | | | | starting at a |
| | | | | | promoter/exon 1 |
| | | | | | located in intron 44. |
| | | | | | Dp140 transcripts |
| | | | | | have along (1 kb) 5′ |
| | | | | | UTR since |
| | | | | | translation is initiated |
| | | | | | in exon 51 |
| | | | | | (corresponding to aa |
| | | | | | 2461 of dystrophin). |
| | | | | | In addition to the |
| | | | | | alternative promoter |
| | | | | | and exon 1, |
| | | | | | differential splicing |
| | | | | | of exons 71-74 and |
| | | | | | 78 produces at least |
| | | | | | five Dp140 isoforms. |
| | | | | | Of these, this |
| | | | | | transcript (Dp140) |
| | | | | | contains all of the |
| | | | | | exons. |
| Dystrophin | NM_004014.2 | 396 | NP_004005.1 | 397 | Transcript Variant: |
| Dp116 | | | | | transcript Dp116 |
| isoform | | | | | uses exons 56-79, |
| | | | | | starting from a |
| | | | | | promoter/exon 1 |
| | | | | | within intron 55. As |
| | | | | | a result, the Dp116 |
| | | | | | isoform contains a |
| | | | | | unique N-terminal |
| | | | | | MLHRKTYHVK aa |
| | | | | | sequence, instead of |
| | | | | | aa 1-2739 of |
| | | | | | dystrophin. |
| | | | | | Differential splicing |
| | | | | | produces several |
| | | | | | Dp116-subtypes. The |
| | | | | | Dp116 isoform is |
| | | | | | also known as S- |
| | | | | | dystrophin or apo- |
| | | | | | dystrophin-2. |
| Dystrophin | NM_004015.2 | 398 | NP_004006.1 | 399 | Transcript Variant: |
| Dp71 | | | | | Dp71 transcripts use |
| isoform | | | | | exons 63-79 with a |
| | | | | | novel 80- to 100-nt |
| | | | | | exon containing an |
| | | | | | ATG start site for a |
| | | | | | new coding sequence |
| | | | | | of 17 nt. The short |
| | | | | | coding sequence is |
| | | | | | in-frame with the |
| | | | | | consecutive |
| | | | | | dystrophin sequence |
| | | | | | from exon 63. |
| | | | | | Differential splicing |
| | | | | | of exons 71 and 78 |
| | | | | | produces at least four |
| | | | | | Dp71 isoforms. Of |
| | | | | | these, this transcript |
| | | | | | (Dp71) includes both |
| | | | | | exons 71 and 78. |
| Dystrophin | NM_004016.2 | 400 | NP_004007.1 | 401 | Transcript Variant: |
| Dp71b | | | | | Dp71 transcripts use |
| isoform | | | | | exons 63-79 with a |
| | | | | | novel 80- to 100-nt |
| | | | | | exon containing an |
| | | | | | ATG start site for a |
| | | | | | new coding sequence |
| | | | | | of 17 nt. The short |
| | | | | | coding sequence is |
| | | | | | in-frame with the |
| | | | | | consecutive |
| | | | | | dystrophin sequence |
| | | | | | from exon 63. |
| | | | | | Differential splicing |
| | | | | | of exons 71 and 78 |
| | | | | | produces at least four |
| | | | | | Dp71 isoforms. Of |
| | | | | | these, this transcript |
| | | | | | (Dp71b) lacks exon |
| | | | | | 78 and encodes a |
| | | | | | protein with a |
| | | | | | different C-terminus |
| | | | | | than Dp71 and |
| | | | | | Dp71a isoforms. |
| Dystrophin | NM_004017.2 | 402 | NP_004008.1 | 403 | Transcript Variant: |
| Dp71a | | | | | Dp71 transcripts use |
| isoform | | | | | exons 63-79 with a |
| | | | | | novel 80- to 100-nt |
| | | | | | exon containing an |
| | | | | | ATG start site for a |
| | | | | | new coding sequence |
| | | | | | of 17 nt. The short |
| | | | | | coding sequence is |
| | | | | | in-frame with the |
| | | | | | consecutive |
| | | | | | dystrophin sequence |
| | | | | | from exon 63. |
| | | | | | Differential splicing |
| | | | | | of exons 71 and 78 |
| | | | | | produces at least four |
| | | | | | Dp71 isoforms. Of |
| | | | | | these, this transcript |
| | | | | | (Dp71a) lacks exon |
| | | | | | 71. |
| Dystrophin | NM_004018.2 | 404 | NP_004009.1 | 405 | Transcript Variant: |
| Dp71ab | | | | | Dp71 transcripts use |
| isoform | | | | | exons 63-79 with a |
| | | | | | novel 80- to 100-nt |
| | | | | | exon containing an |
| | | | | | ATG start site for a |
| | | | | | new coding sequence |
| | | | | | of 17 nt. The short |
| | | | | | coding sequence is |
| | | | | | in-frame with the |
| | | | | | consecutive |
| | | | | | dystrophin sequence |
| | | | | | from exon 63. |
| | | | | | Differential splicing |
| | | | | | of exons 71 and 78 |
| | | | | | produces at least four |
| | | | | | Dp71 isoforms. Of |
| | | | | | these, this transcript |
| | | | | | (Dp71ab) lacks both |
| | | | | | exons 71 and 78 and |
| | | | | | encodes a protein |
| | | | | | with a C-terminus |
| | | | | | like isoform Dp71b. |
| Dystrophin | NM_004019.2 | 406 | NP_004010.1 | 407 | Transcript Variant: |
| Dp40 | | | | | transcript Dp40 uses |
| isoform | | | | | exons 63-70. The 5′ |
| | | | | | UTR and encoded |
| | | | | | first 7 aa are identical |
| | | | | | to that in transcript |
| | | | | | Dp71, but the stop |
| | | | | | codon lies at the |
| | | | | | splice junction of the |
| | | | | | exon/intron 70. The |
| | | | | | 3′ UTR includes nt |
| | | | | | from intron 70 which |
| | | | | | includes an |
| | | | | | alternative |
| | | | | | polyadenylation site. |
| | | | | | The Dp40 isoform |
| | | | | | lacks the normal C- |
| | | | | | terminal end of full- |
| | | | | | length dystrophin (aa |
| | | | | | 3409-3685). |
| Dystrophin | NM_004020.3 | 408 | NP_004011.2 | 409 | Transcript Variant: |
| Dp140c | | | | | Dp140 transcripts |
| isoform | | | | | use exons 45-79, |
| | | | | | starting at a |
| | | | | | promoter/exon 1 |
| | | | | | located in intron 44. |
| | | | | | Dp140 transcripts |
| | | | | | have along (1 kb) 5′ |
| | | | | | UTR since |
| | | | | | translation is initiated |
| | | | | | in exon 51 |
| | | | | | (corresponding to aa |
| | | | | | 2461 of dystrophin). |
| | | | | | In addition to the |
| | | | | | alternative promoter |
| | | | | | and exon 1, |
| | | | | | differential splicing |
| | | | | | of exons 71-74 and |
| | | | | | 78 produces at least |
| | | | | | five Dp140 isoforms. |
| | | | | | Of these, this |
| | | | | | transcript (Dp140c) |
| | | | | | lacks exons 71-74. |
| Dystrophin | NM_004021.2 | 410 | NP_004012.1 | 411 | Transcript Variant: |
| Dp140b | | | | | Dp140 transcripts |
| isoform | | | | | use exons 45-79, |
| | | | | | starting at a |
| | | | | | promoter/exon 1 |
| | | | | | located in intron 44. |
| | | | | | Dp140 transcripts |
| | | | | | have along (1 kb) 5′ |
| | | | | | UTR since |
| | | | | | translation is initiated |
| | | | | | in exon 51 |
| | | | | | (corresponding to aa |
| | | | | | 2461 of dystrophin). |
| | | | | | In addition to the |
| | | | | | alternative promoter |
| | | | | | and exon 1, |
| | | | | | differential splicing |
| | | | | | of exons 71-74 and |
| | | | | | 78 produces at least |
| | | | | | five Dp140 isoforms. |
| | | | | | Of these, this |
| | | | | | transcript (Dp140b) |
| | | | | | lacks exon 78 and |
| | | | | | encodes a protein |
| | | | | | with a unique C- |
| | | | | | terminus. |
| Dystrophin | NM_004022.2 | 412 | NP_004013.1 | 413 | Transcript Variant: |
| Dp140ab | | | | | Dp140 transcripts |
| isoform | | | | | use exons 45-79, |
| | | | | | starting at a |
| | | | | | promoter/exon 1 |
| | | | | | located in intron 44. |
| | | | | | Dp140 transcripts |
| | | | | | have along (1 kb) 5′ |
| | | | | | UTR since |
| | | | | | translation is initiated |
| | | | | | in exon 51 |
| | | | | | (corresponding to aa |
| | | | | | 2461 of dystrophin). |
| | | | | | In addition to the |
| | | | | | alternative promoter |
| | | | | | and exon 1, |
| | | | | | differential splicing |
| | | | | | of exons 71-74 and |
| | | | | | 78 produces at least |
| | | | | | five Dp140 isoforms. |
| | | | | | Of these, this |
| | | | | | transcript (Dp140ab) |
| | | | | | lacks exons 71 and |
| | | | | | 78 and encodes a |
| | | | | | protein with a unique |
| | | | | | C-terminus. |
| Dystrophin | NM_004023.2 | 414 | NP_004014.1 | 415 | Transcript Variant: |
| Dp140bc | | | | | Dp140 transcripts |
| isoform | | | | | use exons 45-79, |
| | | | | | starting at a |
| | | | | | promoter/exon 1 |
| | | | | | located in intron 44. |
| | | | | | Dp140 transcripts |
| | | | | | have along (1 kb) 5′ |
| | | | | | UTR since |
| | | | | | translation is initiated |
| | | | | | in exon 51 |
| | | | | | (corresponding to aa |
| | | | | | 2461 of dystrophin). |
| | | | | | In addition to the |
| | | | | | alternative promoter |
| | | | | | and exon 1, |
| | | | | | differential splicing |
| | | | | | of exons 71-74 and |
| | | | | | 78 produces at least |
| | | | | | five Dp140 isoforms. |
| | | | | | Of these, this |
| | | | | | transcript (Dp140bc) |
| | | | | | lacks exons 71-74 |
| | | | | | and 78 and encodes a |
| | | | | | protein with a unique |
| | | | | | C-terminus. |
| Dystrophin | XM_006724469.3 | 416 | XP_006724532.1 | 417 | |
| isoform | | | | | |
| X2 | | | | | |
| Dystrophin | XM_011545467.1 | 418 | XP_011543769.1 | 419 | |
| isoform | | | | | |
| X5 | | | | | |
| Dystrophin | XM_006724473.2 | 420 | XP_006724536.1 | 421 | |
| isoform | | | | | |
| X6 | | | | | |
| Dystrophin | XM_006724475.2 | 422 | XP_006724538.1 | 423 | |
| isoform | | | | | |
| X8 | | | | | |
| Dystrophin | XM_017029328.1 | 424 | XP_016884817.1 | 425 | |
| isoform | | | | | |
| X4 | | | | | |
| Dystrophin | XM_006724468.2 | 426 | XP_006724531.1 | 427 | |
| isoform | | | | | |
| X1 | | | | | |
| Dystrophin | XM_017029331.1 | 428 | XP_016884820.1 | 429 | |
| isoform | | | | | |
| X13 | | | | | |
| Dystrophin | XM_006724470.3 | 430 | XP_006724533.1 | 431 | |
| isoform | | | | | |
| X3 | | | | | |
| Dystrophin | XM_006724474.3 | 432 | XP_006724537.1 | 433 | |
| isoform | | | | | |
| X7 | | | | | |
| Dystrophin | XM_011545468.2 | 434 | XP_011543770.1 | 435 | |
| isoform | | | | | |
| X9 | | | | | |
| Dystrophin | XM_017029330.1 | 436 | XP_016884819.1 | 437 | |
| isoform | | | | | |
| X11 | | | | | |
| Dystrophin | XM_017029329.1 | 438 | XP_016884818.1 | 439 | |
| isoform | | | | | |
| X10 | | | | | |
| Dystrophin | XM_011545469.1 | 440 | XP_011543771.1 | 441 | |
| isoform | | | | | |
| X12 | | | | | |

The murine dystrophin protein has the following amino acid sequence (Uniprot Accession No. P11531, SEQ. ID. NO. 786):

| | |
|---|---|
| 1 | MWWVDCYRDV KKTTKWNASK GKHDNSDDGK RDGTGKKKGS TRVHANNVNK ARVKNNVDVN |
| 61 | GSTDVDGNHK TGWNHWVKNV MKTMAGTNSK SWVRSTRNYV NVNTSSWSDG ANAHSHRDDW |
| 121 | NSVVSHSATR HANAKCGKDD VATTYDKKSM YTSVVSAVMR TSSKVTRHHH MHYSTVSAGY |
| 181 | TSSSKRKSYA TAAYVATSDS TSYSHARDKS DSSMTVNDSY TAVSWSADTR AGSNDVVKHA |
| 241 | HGMMDTSHGV GNVGSVGKGK SDAVMNNSRW CRVASMKSKH KVMDNKKDDW TKTRTKKMGD |
| 301 | DKCVHKVDVR VNSTHMVVVV DSSGDHATAA KVGDRWANCR WTDRWVDKWH TCSTWSKDAM |
| 361 | KNTSGKDNMM SSHKSTKDKK KTMKSSNDSA KNKSVTKMWM NARWDNTKKS SASAVTTTST |
| 421 | TTVMTVTMVT TRMVKHAKKR TVDSRKRDVD THSWTRSAVS SAVYRKGNSD KVNAARKAKR |
| 481 | KDASRSAAVM ANGVNASRAS NSRWTCSRVN WYTNTYNMTT TANKTSTTST AKSKCKDVNR |
| 541 | SAKSKKGGMD ADVATNHNHD GVRAKKTDTM RYTMSSRTWS SKSVYSVTYM RGKASSKNGN |
| 601 | YSDTVKMAKK ASCKYSGHWK KSSVSCKHMN KRKNHKTKWM AVDVKWAGDA KKKCRVGDTS |
| 661 | NSVNGGKKSA ASRTRNTWDH CRVYTRKAKA GDKTVSKDSM HWMTAYRDYK TDTAVMKRAK |
| 721 | AKTKVKTTVN SVAHASAAKK TTTNYWCTRN GKCKTVWACW HSYKANKWNV KKTMNVAGTV |
| 781 | SNMHHSNNRA TTDGGVMDNT NSRWRHAVRK KSSAKSHSDK AAYTDKVDAA MAKSDTSHSM |
| 841 | KKHNGKDANR VSDVAKKDVS MKRKANRSKM DVKMHATKSV VSSHCVNYKS SVKSVMVKTG |
| 901 | RVKKTNKDRV TAKHYNGAKV TRKKCKSRKM RKMNVTWAAT DTTKRSAVGM SNDSVAWGKA |
| 961 | TKKKAHKSVT GSKMVGKKTV DKSNSNWAVT SRVWNYKHMT DNTKWHADDS KKKKDKRKAM |
| 1021 | NDMRKVDSTR DAAKMANRGD HCRKVVSNRR AASHRKTGKA SKNSDKAGVN KDNKDMSDNG |
| 1081 | TVNRGDNRTD RKRKKTKHNA KDRSRRKKAS HWYYKRADDK CDKKASRDRK KDRKKKNAVR |
| 1141 | RAGSNGAAMA VTSKRWRSNA RRNAHTHTMV VTTDMDVSYV STYTSHASVD HNTCAKDDKS |
| 1201 | KNKDNSGRDH KKKTAASATS MKVKVAVAMD GKHRMYKRGR DRSVKWRHHY DMKVNWNVKK |
| 1261 | TNNWHAKYKW YKDGGRAVVR TNATGSSKTD VNKGSSRWHD CKARRKRKNV SRDNVWADNA |
| 1321 | TGDKVKARGK NTGGAVVSAR DKKKKTNWKV SRAKGVHKDR DHWSRNYNSA GDKVTVHGKA |
| 1381 | DVRSKGHYKK STVKRKDRSW AVNHRRTKDR AGSTTGASAS TVTVTSVVTK TVSKMSSVAA |
| 1441 | DNRAWTTDWS DRVKSRVMVG DDNMKKATDR RTAANKNKTS NARTTDRRWD VNRRNMKDST |
| 1501 | WAKAVGVRGK DSWKGHTVDA KKTTKAKDRR SVDVANDAKR DYSADDTRKV HMTNNTSWGN |
| 1561 | HKRVSAATHR DKSWTATTAN VDASRKKDSR GVRMKWDGTH TDYHNDNGKR SGSDARRDNM |
| 1621 | NKWSKKSNRS HASSDWKRHS VWKDDSRAGG DAVKNDHRAK RKTKVMSTTV RTGKYRRANV |
| 1681 | TRRKAVNAWD KNRSADWRKD ARAADDKRAV KGSWVGDDSD HKVKARGAKN VNRVNDAHTT |
| 1741 | GSYNSTDNTR WRVAVDRVRH AHRDGASHST SVGWRASNKV YYNHTTTCWD HKMTYSADNN |
| 1801 | VRSAYRTAMK RRKACDSSAA CDADHNKNDM DNCTTYDRHN NVNVCVDMCN WNVYDTGRTG |
| 1861 | RRVSKTGSCK AHDKYRYKVA SSTGCDRRGH DSRGVASGGS NSVRSCANNK AADWMRSMVW |
| 1921 | VHRVAAATAK HAKCNCKCGR YRSKHNYDCS CSGRVAKGHK MHYMVYCTTT SGDVRDAKVK |
| 1981 | NKRTKRYAKH RMGYVTVGDN MTVTNWVDSA ASSSHDDTHS RHYASRAMNS NGSYNDSSNS |
| 2041 | DDHHYCSNDS SRSASSRGRA DNRNAYDRKH HKGSSMMTSS RDAAAKRHKG RARMDHNKSH |
| 2101 | RRAAKVNGTT VSSSTSRSDS SMRVVGSTSS MGDSDTSTGV MNNSSSRGRN AGKMRDTM |

Dystrophin is an important component within muscle tissue that provides structural stability to the dystroglycan complex (DGC) of the cell membrane. While both sexes can carry the mutation, females are rarely affected with the skeletal muscle form of the disease.

Mutations vary in nature and frequency. Large genetic deletions are found in about 60-70% of cases, large duplications are found in about 10% of cases, and point mutants or other small changes account for about 15-30% of cases. Bladen et al. (2015), who examined some 7000 mutations, catalogued a total of 5,682 large mutations (80% of total mutations), of which 4,894 (86%) were deletions (1 exon or larger) and 784 (14%) were duplications (1 exon or larger). There were 1,445 small mutations (smaller than 1 exon, 20% of all mutations), of which 358 (25%) were small deletions and 132 (9%) small insertions, while 199 (14%) affected the splice sites. Point mutations totaled 756 (52% of small mutations) with 726 (50%) nonsense mutations and 30 (2%) missense mutations. Finally, 22 (0.3%) mid-intronic mutations were observed. In addition, mutations were identified within the database that would potentially benefit from novel genetic therapies for DMD including stop codon read-through therapies (10% of total mutations) and exon skipping therapy (80% of deletions and 55% of total mutations).

B. Symptoms Symptoms usually appear in boys between the ages of 2 and 3 and may be visible in early infancy. Even though symptoms do not appear until early infancy, laboratory testing can identify children who carry the active mutation at birth. Progressive proximal muscle weakness of the legs and pelvis associated with loss of muscle mass is observed first. Eventually this weakness spreads to the arms, neck, and other areas. Early signs may include pseudohypertrophy (enlargement of calf and deltoid muscles), low endurance, and difficulties in standing unaided or inability to ascend staircases. As the condition progresses, muscle tissue experiences wasting and is eventually replaced by fat and fibrotic tissue (fibrosis). By age 10, braces may be required to aid in walking but most patients are wheelchair dependent by age 12. Later symptoms may include abnormal bone development that lead to skeletal deformities, including curvature of the spine. Due to progressive deterioration of muscle, loss of movement occurs, eventually leading to paralysis. Intellectual impairment may or may not be present but if present, does not progressively worsen as the child ages. The average life expectancy for males afflicted with DMD is around 25.

The main symptom of Duchenne muscular dystrophy, a progressive neuromuscular disorder, is muscle weakness associated with muscle wasting with the voluntary muscles being first affected, especially those of the hips, pelvic area, thighs, shoulders, and calves. Muscle weakness also occurs later, in the arms, neck, and other areas. Calves are often enlarged. Symptoms usually appear before age 6 and may appear in early infancy. Other physical symptoms are:
Awkward manner of walking, stepping, or running—(patients tend to walk on their forefeet, because of an increased calf muscle tone. Also, toe walking is a compensatory adaptation to knee extensor weakness.)
Frequent falls
Fatigue
Difficulty with motor skills (running, hopping, jumping)
Lumbar hyperlordosis, possibly leading to shortening of the hip-flexor muscles. This has an effect on overall posture and a manner of walking, stepping, or running.
Muscle contractures of Achilles tendon and hamstrings impair functionality because the muscle fibers shorten and fibrose in connective tissue
Progressive difficulty walking
Muscle fiber deformities
Pseudohypertrophy (enlarging) of tongue and calf muscles. The muscle tissue is eventually replaced by fat and connective tissue, hence the term pseudohypertrophy.
Higher risk of neurobehavioral disorders (e.g., ADHD), learning disorders (dyslexia), and non-progressive weaknesses in specific cognitive skills (in particular short-term verbal memory), which are believed to be the result of absent or dysfunctional dystrophin in the brain.
Eventual loss of ability to walk (usually by the age of 12)
Skeletal deformities (including scoliosis in some cases)
Trouble getting up from lying or sitting position

The condition can often be observed clinically from the moment the patient takes his first steps, and the ability to walk usually completely disintegrates between the time the patient is 9 to 12 years of age. Most men affected with DMD become essentially “paralyzed from the neck down” by the age of 21. Muscle wasting begins in the legs and pelvis, then progresses to the muscles of the shoulders and neck, followed by loss of arm muscles and respiratory muscles. Calf muscle enlargement (pseudohypertrophy) is quite obvious. Cardiomyopathy particularly (dilated cardiomyopathy) is common, but the development of congestive heart failure or arrhythmia (irregular heartbeat) is only occasional.

A positive Gowers' sign reflects the more severe impairment of the lower extremities muscles. The child helps himself to get up with upper extremities: first by rising to stand on his arms and knees, and then “walking” his hands up his legs to stand upright. Affected children usually tire more easily and have less overall strength than their peers. Creatine kinase (CPK-MM) levels in the bloodstream are extremely high. An electromyography (EMG) shows that weakness is caused by destruction of muscle tissue rather than by damage to nerves. Genetic testing can reveal genetic errors in the Xp21 gene. A muscle biopsy (immunohistochemistry or immunoblotting) or genetic test (blood test) confirms the absence of dystrophin, although improvements in genetic testing often make this unnecessary.

Other symptoms include:
Abnormal heart muscle (cardiomyopathy)
Congestive heart failure or irregular heart rhythm (arrhythmia)
Deformities of the chest and back (scoliosis)
Enlarged muscles of the calves, buttocks, and shoulders (around age 4 or 5). These muscles are eventually replaced by fat and connective tissue (pseudohypertrophy).
Loss of muscle mass (atrophy)
Muscle contractures in the heels, legs
Muscle deformities
Respiratory disorders, including pneumonia and swallowing with food or fluid passing into the lungs (in late stages of the disease)

C. Causes

Duchenne muscular dystrophy (DMD) is caused by a mutation of the dystrophin gene at locus Xp21, located on the short arm of the X chromosome. Dystrophin is responsible for connecting the cytoskeleton of each muscle fiber to the underlying basal lamina (extracellular matrix), through a protein complex containing many subunits. The absence of dystrophin permits excess calcium to penetrate the sarcolemma (the cell membrane). Alterations in calcium and signaling pathways cause water to enter into the mitochondria, which then burst.

In skeletal muscle dystrophy, mitochondrial dysfunction gives rise to an amplification of stress-induced cytosolic calcium signals and an amplification of stress-induced reactive-oxygen species (ROS) production. In a complex cascading process that involves several pathways and is not clearly understood, increased oxidative stress within the cell damages the sarcolemma and eventually results in the death of the cell. Muscle fibers undergo necrosis and are ultimately replaced with adipose and connective tissue.

DMD is inherited in an X-linked recessive pattern. Females will typically be carriers for the disease while males will be affected. Typically, a female carrier will be unaware they carry a mutation until they have an affected son. The son of a carrier mother has a 50% chance of inheriting the defective gene from his mother. The daughter of a carrier mother has a 50% chance of being a carrier and a 50% chance of having two normal copies of the gene. In all cases, an unaffected father will either pass a normal Y to his son or a normal X to his daughter. Female carriers of an X-linked recessive condition, such as DMD, can show symptoms depending on their pattern of X-inactivation.

Exon deletions preceding exon 51 of the human DMD gene, which disrupt the open reading frame (ORF) by juxtaposing out of frame exons, represent the most common type of human DMD mutation. Skipping of exon 51 can, in principle, restore the DMD ORF in 13% of DMD patients with exon deletions.

Duchenne muscular dystrophy has an incidence of 1 in 5000 male infants. Mutations within the dystrophin gene can either be inherited or occur spontaneously during germline transmission. A table of exemplary but non-limiting mutations and corresponding models are set forth below:

| Deletion, small insertion and | |
|---|---|
| nonsense mutations | Name of Mouse Model |
| Exon 44 | ΔEx44 |
| Exon 52 | ΔEx52 |
| Exon 43 | ΔEx43 |

D. Diagnosis

Genetic counseling is advised for people with a family history of the disorder. Duchenne muscular dystrophy can be detected with about 95% accuracy by genetic studies performed during pregnancy.

DNA test. The muscle-specific isoform of the dystrophin gene is composed of 79 exons, and DNA testing and analysis can usually identify the specific type of mutation of the exon or exons that are affected. DNA testing confirms the diagnosis in most cases.

Muscle biopsy. If DNA testing fails to find the mutation, a muscle biopsy test may be performed. A small sample of muscle tissue is extracted (usually with a scalpel instead of a needle) and a dye is applied that reveals the presence of dystrophin. Complete absence of the protein indicates the condition.

Over the past several years DNA tests have been developed that detect more of the many mutations that cause the condition, and muscle biopsy is not required as often to confirm the presence of Duchenne's.

Prenatal tests. DMD is carried by an X-linked recessive gene. Males have only one X chromosome, so one copy of the mutated gene will cause DMD. Fathers cannot pass X-linked traits on to their sons, so the mutation is transmitted by the mother.

If the mother is a carrier, and therefore one of her two X chromosomes has a DMD mutation, there is a 50% chance that a female child will inherit that mutation as one of her two X chromosomes, and be a carrier. There is a 50% chance that a male child will inherit that mutation as his one X chromosome, and therefore have DMD.

Prenatal tests can tell whether an unborn child has the most common mutations. There are many mutations responsible for DMD, and some have not been identified, so genetic testing only works when family members with DMD have a mutation that has been identified.

Prior to invasive testing, determination of the fetal sex is important; while males are sometimes affected by this X-linked disease, female DMD is extremely rare. This can be achieved by ultrasound scan at 16 weeks or more recently by free fetal DNA testing. Chorion villus sampling (CVS) can be done at 11-14 weeks, and has a 1% risk of miscarriage. Amniocentesis can be done after 15 weeks, and has a 0.5% risk of miscarriage. Fetal blood sampling can be done at about 18 weeks. Another option in the case of unclear genetic test results is fetal muscle biopsy.

E. Treatment There is no current cure for DMD, and an ongoing medical need has been recognized by regulatory authorities. Phase 1-2a trials with exon skipping treatment for certain mutations have halted decline and produced small clinical improvements in walking. Treatment is generally aimed at controlling the onset of symptoms to maximize the quality of life, and include the following:
Corticosteroids such as prednisolone and deflazacort increase energy and strength and defer severity of some symptoms.
Randomized control trials have shown that beta-2-agonists increase muscle strength but do not modify disease progression. Follow-up time for most RCTs on beta2-agonists is only around 12 months and hence results cannot be extrapolated beyond that time frame.
Mild, non jarring physical activity such as swimming is encouraged. Inactivity (such as bed rest) can worsen the muscle disease.
Physical therapy is helpful to maintain muscle strength, flexibility, and function.
Orthopedic appliances (such as braces and wheelchairs) may improve mobility and the ability for self-care. Form-fitting removable leg braces that hold the ankle in place during sleep can defer the onset of contractures.
Appropriate respiratory support as the disease progresses is important.

Comprehensive multi-disciplinary care standards/guidelines for DMD have been developed by the Centers for Disease Control and Prevention (CDC), and are available at www.treat-nmd.eu/dmd/care/diagnosis-management-DMD.

DMD generally progresses through five stages, as outlined in Bushby et al., Lancet Neurol., 9(1): 77-93 (2010) and Bushby et al., Lancet Neurol., 9(2): 177-198 (2010), incorporated by reference in their entireties. During the presymptomatic stage, patients typically show developmental delay, but no gait disturbance. During the early ambulatory stage, patients typically show the Gowers' sign, waddling gait, and toe walking. During the late ambulatory stage, patients typically exhibit an increasingly labored gait and begin to lose the ability to climb stairs and rise from the floor. During the early non-ambulatory stage, patients are typically able to self-propel for some time, are able to maintain posture, and may develop scoliosis. During the late non-ambulatory stage, upper limb function and postural maintenance is increasingly limited.

In some embodiments, treatment is initiated in the presymptomatic stage of the disease. In some embodiments, treatment is initiated in the early ambulatory stage. In some embodiments, treatment is initiated in the late ambulatory stage. In embodiments, treatment is initiated during the early non-ambulatory stage. In embodiments, treatment is initiated during the late non-ambulatory stage.

1. Physical Therapy

Physical therapists are concerned with enabling patients to reach their maximum physical potential. Their aim is to:
minimize the development of contractures and deformity by developing a program of stretches and exercises where appropriate
anticipate and minimize other secondary complications of a physical nature by recommending bracing and durable medical equipment
monitor respiratory function and advise on techniques to assist with breathing exercises and methods of clearing secretions

2. Respiration Assistance

Modern “volume ventilators/respirators,” which deliver an adjustable volume (amount) of air to the person with each breath, are valuable in the treatment of people with muscular dystrophy related respiratory problems. The ventilator may require an invasive endotracheal or tracheotomy tube through which air is directly delivered, but, for some people non-invasive delivery through a face mask or mouthpiece is sufficient. Positive airway pressure machines, particularly bi-level ones, are sometimes used in this latter way. The respiratory equipment may easily fit on a ventilator tray on the bottom or back of a power wheelchair with an external battery for portability.

Ventilator treatment may start in the mid to late teens when the respiratory muscles can begin to collapse. If the vital capacity has dropped below 40% of normal, a volume ventilator/respirator may be used during sleeping hours, a time when the person is most likely to be under ventilating (“hypoventilating”). Hypoventilation during sleep is determined by a thorough history of sleep disorder with an oximetry study and a capillary blood gas (See Pulmonary Function Testing). A cough assist device can help with excess mucus in lungs by hyperinflation of the lungs with positive air pressure, then negative pressure to get the mucus up. If the vital capacity continues to decline to less than 30 percent of normal, a volume ventilator/respirator may also be needed during the day for more assistance. The person gradually will increase the amount of time using the ventilator/respirator during the day as needed.

F. Prognosis

Duchenne muscular dystrophy is a progressive disease which eventually affects all voluntary muscles and involves the heart and breathing muscles in later stages. The life expectancy is currently estimated to be around 25, but this varies from patient to patient. Recent advancements in medicine are extending the lives of those afflicted. The Muscular Dystrophy Campaign, which is a leading UK charity focusing on all muscle disease, states that “with high standards of medical care young men with Duchenne muscular dystrophy are often living well into their 30s.”

In rare cases, persons with DMD have been seen to survive into the forties or early fifties, with the use of proper positioning in wheelchairs and beds, ventilator support (via tracheostomy or mouthpiece), airway clearance, and heart medications, if required. Early planning of the required supports for later-life care has shown greater longevity in people living with DMD.

Curiously, in the mdx mouse model of Duchenne muscular dystrophy, the lack of dystrophin is associated with increased calcium levels and skeletal muscle myonecrosis. The intrinsic laryngeal muscles (ILM) are protected and do not undergo myonecrosis. ILM have a calcium regulation system profile suggestive of a better ability to handle calcium changes in comparison to other muscles, and this may provide a mechanistic insight for their unique pathophysiological properties. The ILM may facilitate the development of novel strategies for the prevention and treatment of muscle wasting in a variety of clinical scenarios.

### II. CRISPR SYSTEMS

A. CRISPRs

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

B. Cas Nucleases

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (˜30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. The team demonstrated that they could disable one or both sites while preserving Cas9's ability to locate its target DNA. tracrRNA and spacer RNA can be combined into a “single-guide RNA” molecule that, mixed with Cas9, can find and cut the correct DNA targets. and Such synthetic guide RNAs are able to be used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Wang et al. (2013) showed that coinjection of Cas9 mRNA and sgRNAs into the germline (zygotes) generated nice with mutations. Delivery of Cas9 DNA sequences also is contemplated.

The systems CRISPR/Cas are separated into three classes. Class 1 uses several Cas proteins together with the CRISPR RNAs (crRNA) to build a functional endonuclease. Class 2 CRISPR systems use a single Cas protein with a crRNA. Cpf1 has been recently identified as a Class II, Type V CRISPR/Cas systems containing a 1,300 amino acid protein. See also U.S. Patent Publication 2014/0068797, which is incorporated by reference in its entirety.

In some embodiments, the compositions of the disclosure include a small version of a Cas9 from the bacterium Staphylococcus aureus (UniProt Accession No. J7RUA5). The small version of the Cas9 provides advantages over wild type or full length Cas9. In some embodiments the Cas9 is a spCas9 (AddGene).

C. Cpf1 Nucleases

Clustered Regularly Interspaced Short Palindromic Repeats from Prevotella and Francisella 1 or CRISPR/Cpf1 is a DNA-editing technology which shares some similarities with the CRISPR/Cas9 system. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations.

Cpf1 appears in many bacterial species. The ultimate Cpf1 endonuclease that was developed into a tool for genome editing was taken from one of the first 16 species known to harbor it.

In embodiments, the Cpf1 is a Cpf1 enzyme from Acidaminococcus (species BV3L6, UniProt Accession No. U2UMQ6; SEQ ID NO. 442), having the sequence set forth below:

| | |
|---|---|
| 1 | mtqfegftnl yqvsktlrfe lipqgktlkh iqeqgfieed karndhykel kpiidriykt |
| 61 | yadqclqlvq ldwenlsaai dsyrkektee trnalieeqa tyrnaihdyf igrtdnltda |
| 121 | inkrhaeiyk glfkaelfng kvlkqlgtvt ttehenallr sfdkfttyfs gfyenrknvf |
| 181 | saedistaip hrivqdnfpk fkenchiftr litavpslre hfenvkkaig ifvstsieev |
| 241 | fsfpfynqll tqtqidlynq llggisreag tekikglnev lnlaiqknde tahiiaslph |
| 301 | rfiplfkqil sdrntlsfil eefksdeevi qsfckyktll rnenvletae alfnelnsid |
| 361 | lthifishkk letissalcd hwdtlrnaly erriseltgk itksakekvq rslkhedinl |
| 421 | qeiisaagke lseafkqkts eilshahaal dqplpttlkk qeekeilksq ldsllglyhl |
| 481 | ldwfavdesn evdpefsarl tgiklemeps lsfynkarny atkkpysvek fklnfqmptl |
| 541 | asgwdvnkek nngailfvkn glyylgimpk qkgrykalsf eptektsegf dkmyydyfpd |
| 601 | aakmipkcst qlkavtahfq thttpillsn nfiepleitk eiydlnnpek epkkfqtaya |
| 661 | kktgdqkgyr ealckwidft rdflskytkt tsidlsslrp ssqykdlgey yaelnpllyh |
| 721 | isfqriaeke imdavetgkl ylfqiynkdf akghhgkpnl htlywtglfs penlaktsik |
| 781 | lngqaelfyr pksrmkrmah rlgekmlnkk lkdqktpipd tlyqelydyv nhrlshdlsd |
| 841 | earallpnvi tkevsheiik drrftsdkff fhvpitlnyq aanspskfnq rvnaylkehp |
| 901 | etpiigidrg ernliyitvi dstgkileqr slntiqqfdy qkkldnreke rvaarqawsv |
| 961 | vgtikdlkqg ylsqviheiv dlmihyqavy vlenlnfgfk skrtgiaeka vyqqfekmli |
| 1021 | dklnclvlkd ypaekvggvl npyqltdqft sfakmgtqsg flfyvpapyt skidpltgfv |
| 1081 | dpfvwktikn hesrkhfleg fdflhydvkt gdfilhfkmn rnlsfqrglp gfmpawdivf |
| 1141 | eknetqfdak gtpfiagkri vpvienhrft gryrdlypan elialleekg ivfrdgsnil |
| 1201 | pkllenddsh aidtmvalir svlqmrnsna atgedyinsp vrdlngvcfd srfqnpewpm |
| 1261 | dadangayhi alkgqlllnh lkeskdlklq ngisnqdwla yiqelrn |

In some embodiments, the Cpf1 is a Cpf1 enzyme from Lachnospiraceae (species ND2006, UniProt Accession No. A0A182DWE3; SEQ ID NO. 443), having the sequence set forth below:

| | |
|---|---|
| 1 | AASKLEKFTN CYSLSKTLRF KAIPVGKTQE NIDNKRLLVE DEKRAEDYKG VKKLLDRYYL |
| 61 | SFINDVLHSI KLKNLNNYIS LFRKKTRTEK ENKELENLEI NLRKEIAKAF KGAAGYKSLF |
| 121 | KKDIIETILP EAADDKDEIA LVNSFNGFTT AFTGFFDNRE NMFSEEAKST SIAFRCINEN |
| 181 | LTRYISNMDI FEKVDAIFDK HEVQEIKEKI LNSDYDVEDF FEGEFFNFVL TQEGIDVYNA |
| 241 | IIGGFVTESG EKIKGLNEYI NLYNAKTKQA LPKFKPLYKQ VLSDRESLSF YGEGYTSDEE |
| 301 | VLEVFRNTLN KNSEIFSSIK KLEKLFKNFD EYSSAGIFVK NGPAISTISK DIFGEWNLIR |
| 361 | DKWNAEYDDI HLKKKAVVTE KYEDDRRKSF KKIGSFSLEQ LQEYADADLS VVEKLKEIII |
| 421 | QKVDEIYKVY GSSEKLFDAD FVLEKSLKKN DAVVAIMKDL LDSVKSFENY IKAFFGEGKE |
| 481 | TNRDESFYGD FVLAYDILLK VDHIYDAIRN YVTQKPYSKD KFKLYFQNPQ FMGGWDKDKE |
| 541 | TDYRATILRY GSKYYLAIMD KKYAKCLQKI DKDDVNGNYE KINYKLLPGP NKMLPKVFFS |
| 601 | KKWMAYYNPS EDIQKIYKNG TFKKGDMFNL NDCHKLIDFF KDSISRYPKW SNAYDFNFSE |
| 661 | TEKYKDIAGF YREVEEQGYK VSFESASKKE VDKLVEEGKL YMFQIYNKDF SDKSHGTPNL |
| 721 | HTMYFKLLFD ENNHGQIRLS GGAELFMRRA SLKKEELVVH PANSPIANKN PDNPKKTTTL |
| 781 | SYDVYKDKRF SEDQYELHIP IAINKCPKNI FKINTEVRVL LKHDDNPYVI GIDRGERNLL |
| 841 | YIVVVDGKGN IVEQYSLNEI INNFNGIRIK TDYHSLLDKK EKERFEARQN WTSIENIKEL |
| 901 | KAGYISQVVH KICELVEKYD AVIALEDLNS GFKNSRVKVE KQVYQKFEKM LIDKLNYMVD |
| 961 | KKSNPCATGG ALKGYQITNK FESFKSMSTQ NGFIFYIPAW LTSKIDPSTG FVNLLKTKYT |
| 1021 | SIADSKKFIS SFDRIMYVPE EDLFEFALDY KNFSRTDADY IKKWKLYSYG NRIRIFAAAK |
| 1081 | KNNVFAWEEV CLTSAYKELF NKYGINYQQG DIRALLCEQS DKAFYSSFMA LMSLMLQMRN |
| 1141 | SITGRTDVDF LISPVKNSDG IFYDSRNYEA QENAILPKNA DANGAYNIAR KVLWAIGQFK |
| 1201 | KAEDEKLDKV KIAISNKEWL EYAQTSVK |

In some embodiments, the Cpf1 is codon optimized for expression in mammalian cells. In some embodiments, the Cpf1 is codon optimized for expression in human cells or mouse cells.

The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9. Furthermore, Cpf1 does not have a HNH endonuclease domain, and the N-terminal of Cpf1 does not have the alpha-helical recognition lobe of Cas9.

Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Database searches suggest the abundance of Cpf1-family proteins in many bacterial species.

Functional Cpf1 does not require a tracrRNA. Therefore, functional Cpf1 gRNAs of the disclosure may comprise or consist of a crRNA. This benefits genome editing because Cpf1 is not only a smaller nuclease than Cas9, but also it has a smaller sgRNA molecule (approximately half as many nucleotides as Cas9).

The Cpf1-gRNA (e.g. Cpf1-crRNA) complex cleaves target DNA or RNA by identification of a protospacer adjacent motif 5′-YTN-3′ (where “Y” is a pyrimidine and “N” is any nucleobase) or 5′-TTN-3′, in contrast to the G-rich PAM targeted by Cas9. After identification of PAM, Cpf1 introduces a sticky-end-like DNA double-stranded break of 4 or 5 nucleotides overhang.

The CRISPR/Cpf1 system comprises or consists of a Cpf1 enzyme and a guide RNA that finds and positions the complex at the correct spot on the double helix to cleave target DNA. In its native bacterial hosts, CRISPR/Cpf1 systems activity has three stages:

Adaptation, during which Cas1 and Cas2 proteins facilitate the adaptation of small fragments of DNA into the CRISPR array; Formation of crRNAs: processing of pre-cr-RNAs producing of mature crRNAs to guide the Cas protein; and

Interference, in which the Cpf1 is bound to a crRNA to form a binary complex to identify and cleave a target DNA sequence.

This system has been modified to utilize non-naturally occurring crRNAs, which guide Cpf1 to a desired target sequence in a non-bacterial cell, such as a mammalian cell.

D. gRNA

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type III promoter U6. Synthetic gRNAs are slightly over 100 bp at the minimum length and contain a portion which targets the 20 protospacer nucleotides immediately preceding the PAM sequence NGG; gRNAs do not contain a PAM sequence.

In some embodiments, the gRNA targets a site within a wildtype dystrophin gene. In some embodiments, the gRNA targets a site within a mutant dystrophin gene. In some embodiments, the gRNA targets a dystrophin intron. In some embodiments, the gRNA targets a dystrophin exon. In some embodiments, the gRNA targets a site in a dystrophin exon that is expressed and is present in one or more of the dystrophin isoforms shown in Table 1. In embodiments, the gRNA targets a dystrophin splice site. In some embodiments, the gRNA targets a splice donor site on the dystrophin gene. In embodiments, the gRNA targets a splice acceptor site on the dystrophin gene.

In embodiments, the guide RNA targets a mutant DMD exon. In some embodiments, the mutant exon is exon 23 or 51. In some embodiments, the guide RNA targets at least one of exons 1, 23, 41, 44, 46, 47, 48, 49, 50, 51, 52, 53, 54, or 55 of the dystrophin gene. In embodiments, the guide RNA targets at least one of introns 44, 45, 50, 51, 52, 53, 54, or 55 of the dystrophin gene. In preferred embodiments, the guide RNAs are designed to induce skipping of exon 51 or exon 23. In embodiments, the gRNA is targeted to a splice acceptor site of exon 51 or exon 23.

Suitable gRNAs for use in various compositions and methods disclosed herein are provided as SEQ ID NOs. 448-770. (Table E). In preferred embodiments, the gRNA is selected from any one of SEQ ID No. 448 to SEQ ID No. 770.

In some embodiments, gRNAs of the disclosure comprise a sequence that is complementary to a target sequence within a coding sequence or a non-coding sequence corresponding to the DMD gene, and, therefore, hybridize to the target sequence. In some embodiments, gRNAs for Cpf1 comprise a single crRNA containing a direct repeat scaffold sequence followed by 24 nucleotides of guide sequence. In some embodiments, a “guide” sequence of the crRNA comprises a sequence of the gRNA that is complementary to a target sequence. In some embodiments, crRNA of the disclosure comprises a sequence of the gRNA that is not complementary to a target sequence. “Scaffold” sequences of the disclosure link the gRNA to the Cpf1 polypeptide. “Scaffold” sequences of the disclosure are not equivalent to a tracrRNA sequence of a gRNA-Cas9 construct.

E. Cas9 Versus Cpf1

Cas9 requires two RNA molecules to cut DNA while Cpf1 needs one. The proteins also cut DNA at different places, offering researchers more options when selecting an editing site. Cas9 cuts both strands in a DNA molecule at the same position, leaving behind ‘blunt’ ends. Cpf1 leaves one strand longer than the other, creating ‘sticky’ ends that are easier to work with. Cpf1 appears to be more able to insert new sequences at the cut site, compared to Cas9. Although the CRISPR/Cas9 system can efficiently disable genes, it is challenging to insert genes or generate a knock-in. Cpf1 lacks tracrRNA, utilizes a T-rich PAM and cleaves DNA via a staggered DNA DSB.

In summary, important differences between Cpf1 and Cas9 systems are that Cpf1 recognizes different PAMs, enabling new targeting possibilities, creates 4-5 nt long sticky ends, instead of blunt ends produced by Cas9, enhancing the efficiency of genetic insertions and specificity during NHEJ or HDR, and cuts target DNA further away from PAM, further away from the Cas9 cutting site, enabling new possibilities for cleaving the DNA.

| Feature | Cas9 | Cpf1 |
|---|---|---|
| Structure | Two RNA required (Or 1 fusion | One RNA required |
| | transcript (crRNA + tracrRNA = | |
| | gRNA) | |
| Cutting | Blunt end cuts | Staggered end cuts |
| mechanism | | |
| Cutting site | Proximal to recognition site | Distal from recognition |
| | | site |
| Target sites | G-rich PAM | T-rich PAM |
| Cell type | Fast growing cells, including | Non-dividing cells, |
| | cancer cells | including nerve cells |

F. CRISPR/Cpf1-Mediated Gene Editing

The first step in editing the DMD gene using CRISPR/Cpf1 is to identify the genomic target sequence. The genomic target for the gRNAs of the disclosure can be any ˜24 nucleotide DNA sequence within the dystrophin gene, provided that the sequence is unique compared to the rest of the genome. In some embodiments, the genomic target sequence corresponds to a sequence within exon 51, exon 45, exon 44, exon 53, exon 46, exon 52, exon 50, exon 43, exon 6, exon 7, exon 8, and/or exon 55 of the human dystrophin gene. In some embodiments, the genomic target sequence is a 5′ or 3′ splice site of exon 51, exon 45, exon 44, exon 53, exon 46, exon 52, exon 50, exon 43, exon 6, exon 7, exon 8, and/or exon 55 of the human dystrophin gene. In some embodiments, the genomic target sequence corresponds to a sequence within an intron immediately upstream or downstream of exon 51, exon 45, exon 44, exon 53, exon 46, exon 52, exon 50, exon 43, exon 6, exon 7, exon 8, and/or exon 55 of the human dystrophin gene. Exemplary genomic target sequences can be found in Table D.

The next step in editing the DMD gene using CRISPR/Cpf1 is to identify all Protospacer Adjacent Motif (PAM) sequences within the genetic region to be targeted. Cpf1 utilizes a T-rich PAM sequence (TTTN, wherein N is any nucleotide). The target sequence must be immediately upstream of a PAM. Once all possible PAM sequences and putative target sites have been identified, the next step is to choose which site is likely to result in the most efficient on-target cleavage. The gRNA targeting sequence needs to match the target sequence, and the gRNA targeting sequence must not match additional sites within the genome. In preferred embodiments, the gRNA targeting sequence has perfect homology to the target with no homology elsewhere in the genome. In some embodiments, a given gRNA targeting sequence will have additional sites throughout the genome where partial homology exists. These sites are called “off-targets” and should be considered when designing a gRNA. In general, off-target sites are not cleaved as efficiently when mismatches occur near the PAM sequence, so gRNAs with no homology or those with mismatches close to the PAM sequence will have the highest specificity. In addition to “off-target activity”, factors that maximize cleavage of the desired target sequence (“on-target activity”) must be considered. It is known to those of skill in the art that two gRNA targeting sequences, each having 100% homology to the target DNA may not result in equivalent cleavage efficiency. In fact, cleavage efficiency may increase or decrease depending upon the specific nucleotides within the selected target sequence. Close examination of predicted on-target and off-target activity of each potential gRNA targeting sequence is necessary to design the best gRNA. Several gRNA design programs have been developed that are capable of locating potential PAM and target sequences and ranking the associated gRNAs based on their predicted on-target and off-target activity (e.g. CRISPRdirect, available at www.crispr.dbcls.jp).

The next step is to synthesize and clone desired gRNAs. Targeting oligos can be synthesized, annealed, and inserted into plasmids containing the gRNA scaffold using standard restriction-ligation cloning. However, the exact cloning strategy will depend on the gRNA vector that is chosen. The gRNAs for Cpf1 are notably simpler than the gRNAs for Cas9, and only consist of a single crRNA containing direct repeat scaffold sequence followed by ˜24 nucleotides of guide sequence. Cpf1 requires a minimum of 16 nucleotides of guide sequence to achieve detectable DNA cleavage, and a minimum of 18 nucleotides of guide sequence to achieve efficient DNA cleavage in vitro. In some embodiments, 20-24 nucleotides of guide sequence is used. The seed region of the Cpf1 gRNA is generally within the first 5 nucleotides on the 5′ end of the guide sequence. Cpf1 makes a staggered cut in the target genomic DNA. In AsCpf1 and LbCpf1, the cut occurs 19 bp after the PAM on the targeted (+) strand, and 23 bp on the other strand.

Each gRNA should then be validated in one or more target cell lines. For example, after the CRISPR and gRNA are delivered to the cell, the genomic target region may be amplified using PCR and sequenced according to methods known to those of skill in the art.

In some embodiments, gene editing may be performed in vitro or ex vivo. In some embodiments, cells are contacted in vitro or ex vivo with a Cpf1 and a gRNA that targets a dystrophin splice site. In some embodiments, the cells are contacted with one or more nucleic acids encoding the Cpf1 and the guide RNA. In some embodiments, the one or more nucleic acids are introduced into the cells using, for example, lipofection or electroporation.

Gene editing may also be performed in zygotes. In embodiments, zygotes may be injected with one or more nucleic acids encoding Cpf1 and a gRNA that targets a dystrophin splice site. The zygotes may subsequently be injected into a host.

In embodiments, the Cpf1 is provided on a vector. In embodiments, the vector contains a Cpf1 sequence derived from a Lachnospiraceae bacterium. See, for example, Uniprot Accession No. A0A182DWE3; SEQ ID NO. 443. In embodiments, the vector contains a Cpf1 sequence derived from an Acidaminococcus bacterium. See, for example, Uniprot Accession No. U2UMQ6; SEQ ID NO. 442. In some embodiments, the Cpf1 sequence is codon optimized for expression in human cells or mouse cells. In some embodiments, the vector further contains a sequence encoding a fluorescent protein, such as GFP, which allows Cpf1-expressing cells to be sorted using fluorescence activated cell sorting (FACS). In some embodiments, the vector is a viral vector such as an adeno-associated viral vector.

In embodiments, the gRNA is provided on a vector. In some embodiments, the vector is a viral vector such as an adeno-associated viral vector. In embodiments, the Cpf1 and the guide RNA are provided on the same vector. In embodiments, the Cpf1 and the guide RNA are provided on different vectors.

In some embodiments, the cells are additionally contacted with a single-stranded DMD oligonucleotide to effect homology directed repair. In some embodiments, small INDELs restore the protein reading frame of dystrophin (“reframing” strategy). When the reframing strategy is used, the cells may be contacted with a single gRNA. In embodiments, a splice donor or splice acceptor site is disrupted, which results in exon skipping and restoration of the protein reading frame (“exon skipping” strategy). When the exon skipping strategy is used, the cells may be contacted with two or more gRNAs.

Efficiency of in vitro or ex vivo Cpf1-mediated DNA cleavage may be assessed using techniques known to those of skill in the art, such as the T7 E1 assay. Restoration of DMD expression may be confirmed using techniques known to those of skill in the art, such as RT-PCR, western blotting, and immunocytochemistry.

In some embodiments, in vitro or ex vivo gene editing is performed in a muscle or satellite cell. In some embodiments, gene editing is performed in iPSC or iCM cells. In embodiments, the iPSC cells are differentiated after gene editing. For example, the iPSC cells may be differentiated into a muscle cell or a satellite cell after editing. In embodiments, the iPSC cells are differentiated into cardiac muscle cells, skeletal muscle cells, or smooth muscle cells. In embodiments, the iPSC cells are differentiated into cardiomyocytes. iPSC cells may be induced to differentiate according to methods known to those of skill in the art.

In some embodiments, contacting the cell with the Cpf1 and the gRNA restores dystrophin expression. In embodiments, cells which have been edited in vitro or ex vivo, or cells derived therefrom, show levels of dystrophin protein that is comparable to wild type cells. In embodiments, the edited cells, or cells derived therefrom, express dystrophin at a level that is 50%, 60%, 70%, 80%, 90%, 95% or any percentage in between of wild type dystrophin expression levels. In embodiments, the cells which have been edited in vitro or ex vivo, or cells derived therefrom, have a mitochondrial number that is comparable to that of wild type cells. In embodiments the edited cells, or cells derived therefrom, have 50%, 60%, 70%, 80%, 90%, 95% or any percentage in between as many mitochondria as wild type cells. In embodiments, the edited cells, or cells derived therefrom, show an increase in oxygen consumption rate (OCR) compared to non-edited cells at baseline.

### III. NUCLEIC ACID DELIVERY

As discussed above, in certain embodiments, expression cassettes are employed to express a transcription factor product, either for subsequent purification and delivery to a cell/subject, or for use directly in a genetic-based delivery approach. Provided herein are expression vectors which contain one or more nucleic acids encoding Cpf1 and at least one DMD guide RNA that targets a dystrophin splice site. In some embodiments, a nucleic acid encoding Cpf1 and a nucleic acid encoding at least one guide RNA are provided on the same vector. In further embodiments, a nucleic acid encoding Cpf1 and a nucleic acid encoding least one guide RNA are provided on separate vectors.

Expression requires that appropriate signals be provided in the vectors, and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

In some embodiments, the Cpf1 constructs of the disclosure are expressed by a muscle-cell specific promoter. This muscle-cell specific promoter may be constitutively active or may be an inducible promoter.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2κb, HSP70, proliferin, tumor necrosis factor, and/or thyroid stimulating hormone a gene. In some embodiments, the inducer is phorbol ester (TFA), heavy metals, glucocorticoids, poly(rI)x, poly(rc), EIA, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Of particular interest are muscle specific promoters. These include the myosin light chain-2 promoter, the α-actin promoter, the troponin 1 promoter; the Na⁺/Ca²⁺ exchanger promoter, the dystrophin promoter, the α7 integrin promoter, the brain natriuretic peptide promoter and the αB-crystallin/small heat shock protein promoter, α-myosin heavy chain promoter and the ANF promoter. In some embodiments, the muscle specific promoter is the CK8 promoter, which has the following sequence (SEQ ID NO: 787):

| | |
|---|---|
| 1 | CTAGACTAGC ATGCTGCCCA TGTAAGGAGG CAAGGCCTGG GGACACCCGA GATGCCTGGT |
| 61 | TATAATTAAC CCAGACATGT GGCTGCCCCC CCCCCCCCAA CACCTGCTGC CTCTAAAAAT |
| 121 | AACCCTGCAT GCCATGTTCC CGGCGAAGGG CCAGCTGTCC CCCGCCAGCT AGACTCAGCA |
| 181 | CTTAGTTTAG GAACCAGTGA GCAAGTCAGC CCTTGGGGCA GCCCATACAA GGCCATGGGG |
| 241 | CTGGGCAAGC TGCACGCCTG GGTCCGGGGT GGGCACGGTG CCCGGGCAAC GAGCTGAAAG |
| 301 | CTCATCTGCT CTCAGGGGCC CCTCCCTGGG GACAGCCCCT CCTGGCTAGT CACACCCTGT |
| 361 | AGGCTCCTCT ATATAACCCA GGGGCACAGG GGCTGCCCTC ATTCTACCAC CACCTCCACA |
| 421 | GCACAGACAG ACACTCAGGA GCCAGCCAGC |

In some embodiments, the muscle-cell cell specific promoter is a variant of the CK8 promoter, called CK8e. The CK8e promoter has the following sequence (SEQ ID NO: 788):

| | |
|---|---|
| 1 | TGCCCATGTA AGGAGGCAAG GCCTGGGGAC ACCCGAGATG CCTGGTTATA ATTAACCCAG |
| 61 | ACATGTGGCT GCCCCCCCCC CCCCAACACC TGCTGCCTCT AAAAATAACC CTGCATGCCA |
| 121 | TGTTCCCGGC GAAGGGCCAG CTGTCCCCCG CCAGCTAGAC TCAGCACTTA GTTTAGGAAC |
| 181 | CAGTGAGCAA GTCAGCCCTT GGGGCAGCCC ATACAAGGCC ATGGGGCTGG GCAAGCTGCA |
| 241 | CGCCTGGGTC CGGGGTGGGC ACGGTGCCCG GGCAACGAGC TGAAAGCTCA TCTGCTCTCA |
| 301 | GGGGCCCCTC CCTGGGGACA GCCCCTCCTG GCTAGTCACA CCCTGTAGGC TCCTCTATAT |
| 361 | AACCCAGGGG CACAGGGGCT GCCCTCATTC TACCACCACC TCCACAGCAC AGACAGACAC |
| 421 | TCAGGAGCCA GCCAGC |

Where a cDNA insert is employed, one will typically desire to include a polyadenylation signal to effect proper polyadenylation of the gene transcript. Any polyadenylation sequence may be employed, such as human growth hormone and SV40 polyadenylation signals. Also contemplated as an element of the expression cassette is a terminator. These elements can serve to enhance message levels and to minimize read through from the cassette into other sequences.

B. 2A Peptide

The inventor utilizes the 2A-like self-cleaving domain from the insect virus Thosea asigna (TaV 2A peptide; SEQ ID NO. 444; EGRGSLLTCGDVEENPGP) (Chang et al., 2009). These 2A-like domains have been shown to function across Eukaryotes and cause cleavage of amino acids to occur co-translationally within the 2A-like peptide domain. Therefore, inclusion of TaV 2A peptide allows the expression of multiple proteins from a single mRNA transcript. Importantly, the domain of TaV when tested in eukaryotic systems has shown greater than 99% cleavage activity. Other acceptable 2A-like peptides include, but are not limited to, equine rhinitis A virus (ERAV) 2A peptide (SEQ ID NO. 445; QCTNYALLKLAGDVESNPGP), porcine teschovirus-1 (PTV1) 2A peptide (SEQ ID NO. 446; ATNFSLLKQAGDVEENPGP) and foot and mouth disease virus (FMDV) 2A peptide (SEQ ID NO. 447; PVKQLLNFDLLKLAGDVESNPGP) or modified versions thereof.

In some embodiments, the 2A peptide is used to express a reporter and a Cfp1 simultaneously. The reporter may be, for example, GFP.

Other self-cleaving peptides that may be used include, but are not limited to nuclear inclusion protein a (Nia) protease, a P1 protease, a 3C protease, a L protease, a 3C-like protease, or modified versions thereof.

C. Delivery of Expression Vectors

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals.

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus.

Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5□-tripartite leader (TPL) sequence which makes them preferred mRNAs for translation.

In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenoviruses of the disclosure are replication defective or at least conditionally replication defective. The adenovirus may be of any of the 42 different known serotypes or subgroups A-F. Adenovirus type 5 of subgroup C is the preferred starting material in order to obtain the conditional replication-defective adenovirus vector for use in the present disclosure.

As stated above, the typical vector according to the present disclosure is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression and vaccine development. Animal studies suggested that recombinant adenovirus could be used for gene therapy. Studies in administering recombinant adenovirus to different tissues include trachea instillation, muscle injection, peripheral intravenous injections and stereotactic inoculation into the brain.

The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5□ and 3□ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses may be used, in which biotinylated antibodies against a retroviral envelope protein and against a specific cell receptor are used. The antibodies are coupled via the biotin components by using streptavidin. Using antibodies against major histocompatibility complex class I and class II antigens, it has been demonstrated the infection of a variety of human cells that bore those surface antigens with an ecotropic virus in vitro (Roux et al., 1989).

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes. Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (see, for example, Markowitz et al., 1988; Hersdorffer et al., 1990).

Other viral vectors may be employed as expression constructs in the present disclosure. Vectors derived from viruses such as vaccinia virus, adeno-associated virus (AAV), and herpesviruses may be employed. They offer several attractive features for various mammalian cells.

In embodiments, the AAV vector is replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof. In some embodiments, the AAV vector is not an AAV9 vector.

In some embodiments, a single viral vector is used to deliver a nucleic acid encoding Cpf1 and at least one gRNA to a cell. In some embodiments, Cpf1 is provided to a cell using a first viral vector and at least one gRNA is provided to the cell using a second viral vector. In order to effect expression of sense or antisense gene constructs, the expression construct must be delivered into a cell. The cell may be a muscle cell, a satellite cell, a mesangioblast, a bone marrow derived cell, a stromal cell or a mesenchymal stem cell. In embodiments, the cell is a cardiac muscle cell, a skeletal muscle cell, or a smooth muscle cell. In embodiments, the cell is a cell in the tibialis anterior, quadriceps, soleus, diaphragm or heart. In some embodiments, the cell is an induced pluripotent stem cell (iPSC) or inner cell mass cell (iCM). In further embodiments, the cell is a human iPSC or a human iCM. In some embodiments, human iPSCs or human iCMs of the disclosure may be derived from a cultured stem cell line, an adult stem cell, a placental stem cell, or from another source of adult or embryonic stem cells that does not require the destruction of a human embryo. Delivery to a cell may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In some embodiments, the expression construct is delivered directly to the liver, skin, and/or muscle tissue of a subject. This may require surgical exposure of the tissue or cells, to eliminate any intervening tissue between the gun and the target organ, i.e., ex vivo treatment.

Again, DNA encoding a particular gene may be delivered via this method and still be incorporated by the present disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. A reagent known as Lipofectamine 2000™ is widely used and commercially available.

In certain embodiments, the liposome may be complexed with a hemagglutinating virus (HVJ), to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin. A synthetic neoglycoprotein, which recognizes the same receptor as ASOR, has been used as a gene delivery vehicle and epidermal growth factor (EGF) has also been used to deliver genes to squamous carcinoma cells.

### IV. METHODS OF MAKING TRANSGENIC MICE

A particular embodiment provides transgenic animals that contain mutations in the dystrophin gene. Also, transgenic animals may express a marker that reflects the production of mutant or normal dystrophin gene product.

In a general aspect, a transgenic animal is produced by the integration of a given construct into the genome in a manner that permits the expression of the transgene using methods discussed above. Methods for producing transgenic animals are generally described by Wagner and Hoppe (U.S. Pat. No. 4,873,191; incorporated herein by reference), and Brinster et al. (1985; incorporated herein by reference).

Typically, the construct is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

DNA for microinjection can be prepared by any means known in the art. For example, DNA for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D® column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA. Other methods for purification of DNA for microinjection known to those of skill in the art may be used.

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by CO.sub.2 asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5.degree. C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

### VI. MOUSE MODELS OF DMD

Provided herein is a novel mouse model of DMD, and methods of making the same. The instant disclosure can be used to produce novel mouse models for various DMD mutations.

In some embodiments, the mice are generated using a CRISPR/Cas9 or a CRISPR/Cpf1 system. In embodiments, a single gRNA is used to delete or modify a target DNA sequence. In embodiments, two or more gRNAs are used to delete or modify a target DNA sequence. In some embodiments, the target DNA sequence is an intron. In some embodiments, the target DNA sequence is an exon. In embodiments, the target DNA is a splice donor or acceptor site.

In embodiments, the mouse may be generated by first contacting a fertilized oocyte with CRISPR/Cas9 elements and two single guide RNA (sgRNA) targeting sequences flanking an exon of murine dystrophin. In some embodiments, the exon is exon 50, and in some embodiments the targeting sequences are intronic regions surrounding exon 50. Contacting the fertilized oocyte with the CRISPR/Cas9 elements and the two sgRNAs leads to excision of the exon, thereby creating a modified oocyte. For example, deletion of exon 50 by CRISPR/Cas9 results in an out of frame shift and a premature stop codon in exon 51. The modified oocyte is then transferred into a recipient female.

In embodiments, the fertilized oocyte is derived from a wildtype mouse. In embodiments, the fertilized oocyte is derived from a mouse whose genome contains an exogenous reporter gene. In some embodiments, the exogenous reporter gene is luciferase. In some embodiments, the exogenous reporter gene is a fluorescent protein such as GFP. In some embodiments, a reporter gene expression cassette is inserted into the 3′ end of the dystrophin gene, so that luciferase is translated in-frame with exon 79 of dystrophin. In some embodiments, a self-cleaving peptide such as protease 2A is engineered at a cleavage site between the dystrophin and the luciferase, so that the reporter will be released from the protein after translation.

In some embodiments, the genetically engineered mice described herein have a mutation in the region between exons 45 to 51 of the dystrophin gene. In embodiments, the genetically engineered mice have a deletion of exon 50 of the dystrophin gene resulting in an out of frame shift and a premature stop codon in exon 51 of the dystrophin gene. Deletions and mutations can be confirmed by methods known to those of skill in the art, such as DNA sequencing.

In some embodiments, the genetically engineered mice have a reporter gene. In some embodiments, the reporter gene is located downstream of and in frame with exon 79 of the dystrophin gene, and upstream of a dystrophin 3′-UTR, wherein the reporter gene is expressed when exon 79 is translated in frame with exon 49. In some embodiments, a protease 2A is engineered at a cleavage site between the proteins, which is auto-catalytically cleaved so that the reporter protein is released from dystrophin after translation. In some embodiments, the reporter gene is green fluorescent protein (GFP). In some embodiments, the reporter gene is luciferase.

In embodiments, the mice do not express the dystrophin protein in one or more tissues, for example in skeletal muscle and/or in the heart. In embodiments, the mice exhibit a significant increase of creatine kinase (CK) levels compared to wildtype mice. Elevated CK levels are a sign of muscle damage.

### V. PHARMACEUTICAL COMPOSITIONS AND DELIVERY METHODS

For clinical applications, pharmaceutical compositions are prepared in a form appropriate for the intended application. Generally, thisentails preparing compositions that are essentially free of pyrogens, as well as other impurities that could be harmful to humans or animals.

Appropriate salts and buffers are used to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Any conventional media or agent that is not incompatible with the active ingredients of the present disclosure, its use in therapeutic compositions may be used. Supplementary active ingredients also can be incorporated into the compositions, provided they do not inactivate the vectors or cells of the compositions.

In some embodiments, the active compositions of the present disclosure include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or by direct injection into muscle tissue. Such compositions are normally administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

In some embodiments, the compositions of the present disclosure are formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like)). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, the Cpf1 and gRNAs described herein may be delivered to the patient using adoptive cell transfer (ACT). In adoptive cell transfer, one or more expression constructs are provided ex vivo to cells which have originated from the patient (autologous) or from one or more individual(s) other than the patient (allogeneic). The cells are subsequently introduced or reintroduced into the patient. Thus, in some embodiments, one or more nucleic acids encoding Cpf1 and a guide RNA that targets a dystrophin splice site are provided to a cell ex vivo before the cell is introduced or reintroduced to a patient.

The following tables provide exemplary primer and genomic targeting sequences for use in connection with the compositions and methods disclosed herein.

TABLE C
PRIMER SEQUENCES

| | Primer Name | Primer Sequence | |
|---|---|---|---|
| Cloning | AgeI-nLbCpf1-F1 | F | tttttttGGaccggtgccaccATGAGCAAGCTGGA (SEQ ID NO: 794) |
| primers for | nLbCpf1-R1 | R | TGGGGTTATAGTAGGCCATCCACTTC (SEQ ID NO: 795) |
| pCpf1-2A-GFP | nLbCpf1-F2 | F | GATGGCCTACTATAACCCCAGCG (SEQ ID NO: 796) |
| | nLbCpf1-R2 | R | GGCATAGTCGGGGACATCATATG (SEQ ID NO: 797) |
| | AgeI-nAsCpf1-F1 | F | tttttttcaggttGGaccggtgccaccATGACACAGTTCGAG (SEQ ID NO: 798) |
| | nAsCpf1-R1 | R | TCCTTCTCAGGATTGTTCAGGTCGTA (SEQ ID NO: 799) |
| | nAsCpf1-F2 | F | CTGAACAATCCTGAGAAGGAGCC (SEQ ID NO: 800) |
| | nAsCpf1-R2 | R | GGCATAGTCGGGGACATCATATG (SEQ ID NO: 801) |
| | nCpf1-2A-GFP-F | F | ATGATGTCCCCGACTATGCCgaattcGGCAGTGGAGAGGG (SEQ ID NO: 802) |
| | nCpf1-2A-GFP-R | R | AGCGAGCTCTAGttagaattcCTTGTACAG (SEQ ID NO: 803) |
| In vitro | T7-Scaffold-F | F | CACCAGCGCTGCTTAATACGACTCACTATAGGGAAAT (SEQ ID NO: 804) |
| transcription | T7-Scaffold-R | R | AGTAGCGCTTCTAGACCCTCACTTCCTACTCAG (SEQ ID NO: 18) |
| of LbCpf1 | T7-nLb-F1 | F | AGAAGAAATATAAGACTCGAGgccaccATGAGCAAGCTGGAGAAGTTTAC (SEQ ID NO: 19) |
| mRNA | T7-nLb-R1 | R | TGGGGTTATAGTAGGCCATCC (SEQ ID NO: 20) |
| | T7-nLB-NLS-F2 | F | GATGGCCTACTATAACCCCAGCG (SEQ ID NO: 10) |
| | T7-nLB-NLS-R2 | R | CCCGCAGAAGGCAGCGTCGACTTAGGCATAGTCGGGGACATCATATG (SEQ ID NO: 21) |
| | T7-nAs-F1 | F | AGAAGAAATATAAGACTCGAGgccaccATGACACAGTTCGAGGGCTTTAC (SEQ ID NO: 22) |
| | T7-nAs-R1 | R | TCCTTCTCAGGATTGTTCAGGTCGTA (SEQ ID NO: 13) |
| | T7-nAs-NLS-F2 | F | CTGAACAATCCTGAGAAGGAGCC (SEQ ID NO: 14) |
| | T7-nAs-NLS-R2 | R | CCCGCAGAAGGCAGCGTCGACTTAGGCATAGTCGGGGACATCATATG (SEQ ID NO: 21) |
| Human DMD | nLb-DMD-E51-g1-Top | F | CACCGTAATTTCTACTAAGTGTAGATgCTCCTACTCAGACTGTTACTCTGTTTTTTT |
| Exon 51 gRNA | | | (SEQ ID NO: 23) |
| | nLb-DMD-E51-g1-Bot | R | AAACAAAAAAACAGAGTAACAGTCTGAGTAGGAGcATCTACACTTAGTAGAAATTAC |
| | | | (SEQ ID NO: 24) |
| | nLb-DMD-E51-g2-Top | F | CACCGTAATTTCTACTAAGTGTAGATtaccatgtattgctaaacaaagtaTTTTTTT |
| | | | (SEQ ID NO: 25) |
| | nLb-DMD-E51-g2-Bot | R | AAACAAAAAAAtactttgtttagcaatacatggtaATCTACACTTAGTAGAAATTAC |
| | | | (SEQ ID NO: 26) |
| | nLb-DMD-E51-g3-Top | F | CACCGTAATTTCTACTAAGTGTAGATattgaagagtaacaatttgagccaTTTTTTT |
| | | | (SEQ ID NO: 27) |
| | nLb-DMD-E51-g3-Bot | R | AAACAAAAAAAtggctcaaattgttactcttcaatATCTACACTTAGTAGAAATTAC |
| | | | (SEQ ID NO: 28) |
| | nAs-DMD-E51-g1-Top | F | CACCGTAATTTCTACTCTTGTAGATgCTCCTACTCAGACTGTTACTCTGTTTTTTT |
| | | | (SEQ ID NO: 29) |
| | nAs-DMD-E51-g1-Bot | R | AAACAAAAAAACAGAGTAACAGTCTGAGTAGGAGcATCTACAAGAGTAGAAATTAC |
| | | | (SEQ ID NO: 30) |
| Human DMD | DMD-E51-T7E1-F1 | F | Ttccctggcaaggtctga (SEQ ID NO: 31) |
| Exon 51 T7E1 | DMD-E51-T7E1-R1 | R | ATCCTCAAGGTCACCCACC (SEQ ID NO: 32) |
| Human | Rikens51-RT-PCR-F1 | F | CCCAGAAGAGCAAGATAAACTTGAA (SEQ ID NO: 789) |
| cardiomyo- | Rikens51-RT-PCR-R1 | R | CTCTGTTCCAAATCCTGCATTGT (SEQ ID NO: 33) |
| cytes RT-PCR | | | |
| Human | hmt-ND1-qF1 | F | CGCCACATCTACCATCACCCTC (SEQ ID NO: 790) |
| cardiomyo- | hmt-ND1-qR1 | R | CGGCTAGGCTAGAGGTGGCTA (SEQ ID NO: 791) |
| cytes mtDNA | hLPL-qF1 | F | GAGTATGCAGAAGCCCCGAGTC (SEQ ID NO: 792) |
| copy number | hLPL-qR1 | R | TCAACATGCCCAACTGGTTTCTGG (SEQ ID NO: 793) |
| qPCR | | | |
| Mouse Dmd | nLb-dmd-E23-g1-Top | F | CACCGTAATTTCTACTAAGTGTAGATaggctctgcaaagttctTTGAAAGTTTTTTT |
| Exon 23 | | | (SEQ ID NO: 34) |
| gRNA | nLb-dmd-E23-g1-Bot | R | AAACAAAAAAACTTTCAAagaactttgcagagcctATCTACACTTAGTAGAAATTAC |
| genomic | | | (SEQ ID NO: 35) |
| target | nLb-dmd-E23-g2-Top | F | CACCGTAATTTCTACTAAGTGTAGATAAAGAGCAACAAAATGGCttcaacTTTTTTT |
| sequence | | | (SEQ ID NO: 36) |
| | nLb-dmd-E23-g2-Bot | R | AAACAAAAAAAgttgaaGCCATTTTGTTGCTCTTTATCTACACTTAGTAGAAATTAC |
| | | | (SEQ ID NO: 37) |
| | nLb-mdmd-E23-g2- | F | CACCGTAATTTCTACTAAGTGTAGATAAAGAGCAATAAAATGGCttcaacTTTTTTT |
| | Top | | CACCGTAATTTCTACTAAGTGTAGATAAAGAGCAATAAAATGGCttcaacTTTTTTT |
| | nLb-mdmd-E23-g2- | R | (SEQ ID NO: 38) |
| | Bot | | AAACAAAAAAAgttgaaGCCATTTTATTGCTCTTTATCTACACTTAGTAGAAATTAC |
| | | | (SEQ ID NO: 39) |
| | nLb-dmd-E23-g3-Top | F | CACCGTAATTTCTACTAAGTGTAGATAAAGAACTTTGCAGAGCctcaaaaTTTTTTT |
| | | | (SEQ ID NO: 40) |
| | nLb-dmd-E23-g3-Bot | R | AAACAAAAAAAtttgagGCTCTGCAAAGTTCTTTATCTACACTTAGTAGAAATTAC |
| | | | (SEQ ID NO: 41) |
| | nLb-dmd-I22-g1-Top | F | CACCGTAATTTCTACTAAGTGTAGATctgaatatctatgcattaataactTTTTTTT |
| | | | (SEQ ID NO: 42) |
| | nLb-dmd-I22-g1-Bot | R | AAACAAAAAAAagttattaatgcatagatattcagATCTACACTTAGTAGAAATTAC |
| | | | (SEQ ID NO: 43) |
| | nLb-dmd-I22-g2-Top | F | CACCGTAATTTCTACTAAGTGTAGATtattatattacagggcatattataTTTTTTT |
| | | | (SEQ ID NO: 44) |
| | nLb-dmd-I22-g2-Bot | R | AAACAAAAAAAtataatatgccctgtaatataataATCTACACTTAGTAGAAATTAC |
| | | | (SEQ ID NO: 45) |
| | nLb-dmd-I23-g3-Top | F | CACCGTAATTTCTACTAAGTGTAGATAGgtaagccgaggtttggcctttaTTTTTTT |
| | | | (SEQ ID NO: 46) |
| | nLb-dmd-I23-g3-Bot | R | AAACAAAAAAAtaaaggccaaacctcggcttacCTATCTACACTTAGTAGAAATTAC |
| | | | (SEQ ID NO: 47) |
| | nLb-dmd-I23-g4-Top | F | CACCGTAATTTCTACTAAGTGTAGATcccagagtccttcaaagatattgaTTTTTTT |
| | | | (SEQ ID NO: 48) |
| | nLb-dmd-I23-g4-Bot | R | AAACAAAAAAAtcaatatcttgaaggactctgggATCTACACTTAGTAGAAATTAC |
| | | | (SEQ ID NO: 49) |
| In vitro | T7-Lb-dmd-E23-uF | F | GAATTGTAATACGACTCACTATAGGGTAATTTCTACTAAGTGTAGAT (SEQ ID NO: 50) |
| transcription | T7-Lb-dmd-E23-g1- | R | CTTTCAAagaactttgcagagcctATCTACACTTAGTAGAAATTA (SEQ ID NO: 51) |
| of LbCpf1 | R | | |
| gRNA genomic | T7-Lb-dmd-E23-mg2- | F | GttgaaGCCATTTTATTGCTCTTTATCTACACTTAGTAGAAATTA (SEQ ID NO: 52) |
| target | R | | |
| sequence | T7-Lb-dmd-E23-g3- | R | ttttgagGCTCTGCAAAGTTCTTTATCTACACTTAGTAGAAATTA (SEQ ID NO: 53) |
| | R | | |
| | T7-Lb-dmd-I22-g2- | R | tataatatgccctgtaatataataATCTACACTTAGTAGAAATTACCCTATAGTGAG |
| | R | | (SEQ ID NO: 54) |
| | T7-Lb-dmd-I22-g4- | R | tcaatatctttgaaggactctgggATCTACACTTAGTAGAAATTACCCTATAGTGAG |
| | R | | (SEQ ID NO: 55) |
| Mouse Dmd | Dmd-E23-T7E1-F729 | F | Gagaaacttctgtgatgtgaggacata (SEQ ID NO: 56) |
| Exon 23 | Dmd-E23-T7E1-F1 | R | CAAACCTCGGCTTACCTGAAAT (SEQ ID NO: 57) |
| T7E1 | Dmd-E23-T7E1-R729 | R | Caatatctttgaaggactctgggtaaa (SEQ ID NO: 58) |
| | Dmd-E23-T7E1-R3 | R | Aattaatagaagtcaatgtagggaagg (SEQ ID NO: 59) |

TABLE D
Genomic Target Sequences

| | Guide | | | | |
|---|---|---|---|---|---|
| Targeted gRNA Exon | # | Strand | Genomic Target Sequence* | PAM | SEQ ID NO. |
| Human-Exon 51 | 4 | 1 | tctttttcttcttttttccttttt | tttt | 60 |
| Human-Exon 51 | 5 | 1 | ctttttcttcttttttcctttttG | tttt | 61 |
| Human-Exon 51 | 6 | 1 | tttttcttcttttttcctttttGC | tttc | 62 |
| Human-Exon 51 | 7 | 1 | tcttcttttttcctttttGCAAAA | tttt | 63 |
| Human-Exon 51 | 8 | 1 | cttcttttttcctttttGCAAAAA | tttt | 64 |
| Human-Exon 51 | 9 | 1 | ttcttttttcctttttGCAAAAAC | tttc | 65 |
| Human-Exon 51 | 10 | 1 | ttcctttttGCAAAAACCCAAAAT | tttt | 66 |
| Human-Exon 51 | 11 | 1 | tcctttttGCAAAAACCCAAAATA | tttt | 67 |
| Human-Exon 51 | 12 | 1 | cctttttGCAAAAACCCAAAATAT | tttt | 68 |
| Human-Exon 51 | 13 | 1 | ctttttGCAAAAACCCAAAATATT | tttc | 69 |
| Human-Exon 51 | 14 | 1 | tGCAAAAACCCAAAATATTTTAGC | tttt | 70 |
| Human-Exon 51 | 15 | 1 | GCAAAAACCCAAAATATTTTAGCT | tttt | 71 |
| Human-Exon 51 | 16 | 1 | CAAAAACCCAAAATATTTTAGCTC | tttG | 72 |
| Human-Exon 51 | 17 | 1 | AGCTCCTACTCAGACTGTTACTCT | TTTT | 73 |
| Human-Exon 51 | 18 | 1 | GCTCCTACTCAGACTGTTACTCTG | TTTA | 74 |
| Human-Exon 51 | 19 | −1 | CTTAGTAACCACAGGTTGTGTCAC | TTTC | 75 |
| Human-Exon 51 | 20 | −1 | GAGATGGCAGTTTCCTTAGTAACC | TTTG | 76 |
| Human-Exon 51 | 21 | −1 | TAGTTTGGAGATGGCAGTTTCCTT | TTTC | 77 |
| Human-Exon 51 | 22 | −1 | TTCTCATACCTTCTGCTTGATGAT | TTTT | 78 |
| Human-Exon 51 | 23 | −1 | TCATTTTTTCTCATACCTTCTGCT | TTTA | 79 |
| Human-Exon 51 | 24 | −1 | ATCATTTTTTCTCATACCTTCTGC | TTTT | 80 |
| Human-Exon 51 | 25 | −1 | AAGAAAAACTTCTGCCAACTTTTA | TTTA | 81 |
| Human-Exon 51 | 26 | −1 | AAAGAAAAACTTCTGCCAACTTTT | TTTT | 82 |
| Human-Exon 51 | 27 | 1 | TCTTTAAAATGAAGATTTTCCACC | TTTT | 83 |
| Human-Exon 51 | 28 | 1 | CTTTAAAATGAAGATTTTCCACCA | TTTT | 84 |
| Human-Exon 51 | 29 | 1 | TTTAAAATGAAGATTTTCCACCAA | TTTC | 85 |
| Human-Exon 51 | 30 | 1 | AAATGAAGATTTTCCACCAATCAC | TTTA | 86 |
| Human-Exon 51 | 31 | 1 | CCACCAATCACTTTACTCTCCTAG | TTTT | 87 |
| Human-Exon 51 | 32 | 1 | CACCAATCACTTTACTCTCCTAGA | TTTC | 88 |
| Human-Exon 51 | 33 | 1 | CTCTCCTAGACCATTTCCCACCAG | TTTA | 89 |
| Human-Exon 45 | 1 | −1 | agaaaagattaaacagtgtgctac | tttg | 90 |
| Human-Exon 45 | 2 | −1 | tttgagaaaagattaaacagtgtg | TTTa | 91 |
| Human-Exon 45 | 3 | −1 | atttgagaaaagattaaacagtgt | TTTT | 92 |
| Human-Exon 45 | 4 | −1 | Tatttgagaaaagattaaacagtg | TTTT | 93 |
| Human-Exon 45 | 5 | 1 | atcttttctcaaatAAAAAGACAT | ttta | 94 |
| Human-Exon 45 | 6 | 1 | ctcaaatAAAAAGACATGGGGCTT | tttt | 95 |
| Human-Exon 45 | 7 | 1 | tcaaatAAAAAGACATGGGGCTTC | tttc | 96 |
| Human-Exon 45 | 8 | 1 | TGTTTTGCCTTTTTGGTATCTTAC | TTTT | 97 |
| Human-Exon 45 | 9 | 1 | GTTTTGCCTTTTTGGTATCTTACA | TTTT | 98 |
| Human-Exon 45 | 10 | 1 | TTTTGCCTTTTTGGTATCTTACAG | TTTG | 99 |
| Human-Exon 45 | 11 | 1 | GCCTTTTTGGTATCTTACAGGAAC | TTTT | 100 |
| Human-Exon 45 | 12 | 1 | CCTTTTTGGTATCTTACAGGAACT | TTTG | 101 |
| Human-Exon 45 | 13 | 1 | TGGTATCTTACAGGAACTCCAGGA | TTTT | 102 |
| Human-Exon 45 | 14 | 1 | GGTATCTTACAGGAACTCCAGGAT | TTTT | 103 |
| Human-Exon 45 | 15 | −1 | AGGATTGCTGAATTATTTCTTCCC | TTTG | 104 |
| Human-Exon 45 | 16 | −1 | GAGGATTGCTGAATTATTTCTTCC | TTTT | 105 |
| Human-Exon 45 | 17 | −1 | TGAGGATTGCTGAATTATTTCTTC | TTTT | 106 |
| Human-Exon 45 | 18 | −1 | CTGTAGAATACTGGCATCTGTTTT | TTTC | 107 |
| Human-Exon 45 | 19 | −1 | CCTGTAGAATACTGGCATCTGTTT | TTTT | 108 |
| Human-Exon 45 | 20 | −1 | TCCTGTAGAATACTGGCATCTGTT | TTTT | 109 |
| Human-Exon 45 | 21 | −1 | CAGACCTCCTGCCACCGCAGATTC | TTTG | 110 |
| Human-Exon 45 | 22 | −1 | TGTCTGACAGCTGTTTGCAGACCT | TTTC | 111 |
| Human-Exon 45 | 23 | −1 | CTGTCTGACAGCTGTTTGCAGACC | TTTT | 112 |
| Human-Exon 45 | 24 | −1 | TCTGTCTGACAGCTGTTTGCAGAC | TTTT | 113 |
| Human-Exon 45 | 25 | −1 | TTCTGTCTGACAGCTGTTTGCAGA | TTTT | 114 |
| Human-Exon 45 | 26 | −1 | ATTCCTATTAGATCTGTCGCCCTA | TTTC | 115 |
| Human-Exon 45 | 27 | −1 | CATTCCTATTAGATCTGTCGCCCT | TTTT | 116 |
| Human-Exon 45 | 28 | 1 | AGCAGACTTTTTAAGCTTTCTTTA | TTTT | 117 |
| Human-Exon 45 | 29 | 1 | GCAGACTTTTTAAGCTTTCTTTAG | TTTA | 118 |
| Human-Exon 45 | 30 | 1 | TAAGCTTTCTTTAGAAGAATATTT | TTTT | 119 |
| Human-Exon 45 | 31 | 1 | AAGCTTTCTTTAGAAGAATATTTC | TTTT | 120 |
| Human-Exon 45 | 32 | 1 | AGCTTTCTTTAGAAGAATATTTCA | TTTA | 121 |
| Human-Exon 45 | 33 | 1 | TTTAGAAGAATATTTCATGAGAGA | TTTC | 122 |
| Human-Exon 45 | 34 | 1 | GAAGAATATTTCATGAGAGATTAT | TTTA | 123 |
| Human-Exon 44 | 1 | 1 | TCAGTATAACCAAAAAATATACGC | TTTG | 124 |
| Human-Exon 44 | 2 | 1 | acataatccatctatttttcttga | tttt | 125 |
| Human-Exon 44 | 3 | 1 | cataatccatctatttttcttgat | ttta | 126 |
| Human-Exon 44 | 4 | 1 | tcttgatccatatgcttttACCTG | tttt | 127 |
| Human-Exon 44 | 5 | 1 | cttgatccatatgcttttACCTGC | tttt | 128 |
| Human-Exon 44 | 6 | 1 | ttgatccatatgcttttACCTGCA | tttc | 129 |
| Human-Exon 44 | 7 | −1 | TCAACAGATCTGTCAAATCGCCTG | TTTC | 130 |
| Human-Exon 44 | 8 | 1 | ACCTGCAGGCGATTTGACAGATCT | tttt | 131 |
| Human-Exon 44 | 9 | 1 | CCTGCAGGCGATTTGACAGATCTG | tttA | 132 |
| Human-Exon 44 | 10 | 1 | ACAGATCTGTTGAGAAATGGCGGC | TTTG | 133 |
| Human-Exon 44 | 11 | −1 | TATCATAATGAAAACGCCGCCATT | TTTA | 134 |
| Human-Exon 44 | 12 | 1 | CATTATGATATAAAGATATTTAAT | TTTT | 135 |
| Human-Exon 44 | 13 | −1 | TATTTAGCATGTTCCCAATTCTCA | TTTG | 136 |
| Human-Exon 44 | 14 | −1 | GAAAAAACAAATCAAAGACTTACC | TTTC | 137 |
| Human-Exon 44 | 15 | 1 | ATTTGTTTTTTCGAAATTGTATTT | TTTG | 138 |
| Human-Exon 44 | 16 | 1 | TTTTTTCGAAATTGTATTTATCTT | TTTG | 139 |
| Human-Exon 44 | 17 | 1 | TTCGAAATTGTATTTATCTTCAGC | TTTT | 140 |
| Human-Exon 44 | 18 | 1 | TCGAAATTGTATTTATCTTCAGCA | TTTT | 141 |
| Human-Exon 44 | 19 | 1 | CGAAATTGTATTTATCTTCAGCAC | TTTT | 142 |
| Human-Exon 44 | 20 | 1 | GAAATTGTATTTATCTTCAGCACA | TTTC | 143 |
| Human-Exon 44 | 21 | −1 | AGAAGTTAAAGAGTCCAGATGTGC | TTTA | 144 |
| Human-Exon 44 | 22 | 1 | TCTTCAGCACATCTGGACTCTTTA | TTTA | 145 |
| Human-Exon 44 | 23 | −1 | CATCACCCTTCAGAACCTGATCTT | TTTC | 146 |
| Human-Exon 44 | 24 | 1 | ACTTCTTAAAGATCAGGTTCTGAA | TTTA | 147 |
| Human-Exon 44 | 25 | 1 | GACTGTTGTTGTCATCATTATATT | TTTT | 148 |
| Human-Exon 44 | 26 | 1 | ACTGTTGTTGTCATCATTATATTA | TTTG | 149 |
| Human-Exon 53 | 1 | −1 | AACTAGAATAAAAGGAAAAATAAA | TTTC | 150 |
| Human-Exon 53 | 2 | 1 | CTACTATATATTTATTTTTCCTTT | TTTA | 151 |
| Human-Exon 53 | 3 | 1 | TTTTTCCTTTTATTCTAGTTGAAA | TTTA | 152 |
| Human-Exon 53 | 4 | 1 | TCCTTTTATTCTAGTTGAAAGAAT | TTTT | 153 |
| Human-Exon 53 | 5 | 1 | CCTTTTATTCTAGTTGAAAGAATT | TTTT | 154 |
| Human-Exon 53 | 6 | 1 | CTTTTATTCTAGTTGAAAGAATTC | TTTC | 155 |
| Human-Exon 53 | 7 | 1 | ATTCTAGTTGAAAGAATTCAGAAT | TTTT | 156 |
| Human-Exon 53 | 8 | 1 | TTCTAGTTGAAAGAATTCAGAATC | TTTA | 157 |
| Human-Exon 53 | 9 | −1 | ATTCAACTGTTGCCTCCGGTTCTG | TTTC | 158 |
| Human-Exon 53 | 10 | −1 | ACATTTCATTCAACTGTTGCCTCC | TTTA | 159 |
| Human-Exon 53 | 11 | −1 | CTTTTGGATTGCATCTACTGTATA | TTTT | 160 |
| Human-Exon 53 | 12 | −1 | TGTGATTTTCTTTTGGATTGCATC | TTTC | 161 |
| Human-Exon 53 | 13 | −1 | ATACTAACCTTGGTTTCTGTGATT | TTTG | 162 |
| Human-Exon 53 | 14 | −1 | AAAAGGTATCTTTGATACTAACCT | TTTA | 163 |
| Human-Exon 53 | 15 | −1 | AAAAAGGTATCTTTGATACTAACC | TTTT | 164 |
| Human-Exon 53 | 16 | −1 | TTTTAAAAAGGTATCTTTGATACT | TTTA | 165 |
| Human-Exon 53 | 17 | −1 | ATTTTAAAAAGGTATCTTTGATAC | TTTT | 166 |
| Human-Exon 46 | 1 | −1 | TTAATGCAAACTGGGACACAAACA | TTTG | 167 |
| Human-Exon 46 | 2 | 1 | TAAATTGCCATGTTTGTGTCCCAG | TTTT | 168 |
| Human-Exon 46 | 3 | 1 | AAATTGCCATGTTTGTGTCCCAGT | TTTT | 169 |
| Human-Exon 46 | 4 | 1 | AATTGCCATGTTTGTGTCCCAGTT | TTTA | 170 |
| Human-Exon 46 | 5 | 1 | TGTCCCAGTTTGCATTAACAAATA | TTTG | 171 |
| Human-Exon 46 | 6 | −1 | CAACATAGTTCTCAAACTATTTGT | tttC | 172 |
| Human-Exon 46 | 7 | −1 | CCAACATAGTTCTCAAACTATTTG | 1111 | 173 |
| Human-Exon 46 | 8 | −1 | tCCAACATAGTTCTCAAACTATTT | 1111 | 174 |
| Human-Exon 46 | 9 | −1 | tttCCAACATAGTTCTCAAACTAT | 1111 | 175 |
| Human-Exon 46 | 10 | −1 | ttttCCAACATAGTTCTCAAACTA | tttt | 176 |
| Human-Exon 46 | 11 | −1 | tttttCCAACATAGTTCTCAAACT | 1111 | 177 |
| Human-Exon 46 | 12 | 1 | CATTAACAAATAGTTTGAGAACTA | TTTG | 178 |
| Human-Exon 46 | 13 | 1 | AGAACTATGTTGGaaaaaaaaaTA | TTTG | 179 |
| Human-Exon 46 | 14 | −1 | GTTCTTCTAGCCTGGAGAAAGAAG | TTTT | 180 |
| Human-Exon 46 | 15 | 1 | ATTCTTCTTTCTCCAGGCTAGAAG | TTTT | 181 |
| Human-Exon 46 | 16 | 1 | TTCTTCTTTCTCCAGGCTAGAAGA | TTTA | 182 |
| Human-Exon 46 | 17 | 1 | TCCAGGCTAGAAGAACAAAAGAAT | TTTC | 183 |
| Human-Exon 46 | 18 | −1 | AAATTCTGACAAGATATTCTTTTG | TTTG | 184 |
| Human-Exon 46 | 19 | −1 | CTTTTAGTTGCTGCTCTTTTCCAG | TTTT | 185 |
| Human-Exon 46 | 20 | −1 | AGAAAATAAAATTACCTTGACTTG | TTTG | 186 |
| Human-Exon 46 | 21 | −1 | TGCAAGCAGGCCCTGGGGGATTTG | TTTA | 187 |
| Human-Exon 46 | 22 | 1 | ATTTTCTCAAATCCCCCAGGGCCT | TTTT | 188 |
| Human-Exon 46 | 23 | 1 | TTTTCTCAAATCCCCCAGGGCCTG | TTTA | 189 |
| Human-Exon 46 | 24 | 1 | CTCAAATCCCCCAGGGCCTGCTTG | TTTT | 190 |
| Human-Exon 46 | 25 | 1 | TCAAATCCCCCAGGGCCTGCTTGC | TTTC | 191 |
| Human-Exon 46 | 26 | 1 | TTAATTCAATCATTGGTTTTCTGC | TTTT | 192 |
| Human-Exon 46 | 27 | 1 | TAATTCAATCATTGGTTTTCTGCC | TTTT | 193 |
| Human-Exon 46 | 28 | 1 | AATTCAATCATTGGTTTTCTGCCC | TTTT | 194 |
| Human-Exon 46 | 29 | 1 | ATTCAATCATTGGTTTTCTGCCCA | TTTA | 195 |
| Human-Exon 46 | 30 | −1 | GCAAGGAACTATGAATAACCTAAT | TTTA | 196 |
| Human-Exon 46 | 31 | 1 | CTGCCCATTAGGTTATTCATAGTT | TTTT | 197 |
| Human-Exon 46 | 32 | 1 | TGCCCATTAGGTTATTCATAGTTC | TTTC | 198 |
| Human-Exon 52 | 1 | −1 | TAGAAAACAATTTAACAGGAAATA | TTTA | 199 |
| Human-Exon 52 | 2 | 1 | CTGTTAAATTGTTTTCTATAAACC | TTTC | 200 |
| Human-Exon 52 | 3 | −1 | GAAATAAAAAAGATGTTACTGTAT | TTTA | 201 |
| Human-Exon 52 | 4 | −1 | AGAAATAAAAAAGATGTTACTGTA | TTTT | 202 |
| Human-Exon 52 | 5 | 1 | CTATAAACCCTTATACAGTAACAT | TTTT | 203 |
| Human-Exon 52 | 6 | 1 | TATAAACCCTTATACAGTAACATC | TTTC | 204 |
| Human-Exon 52 | 7 | 1 | TTATTTCTAAAAGTGTTTTGGCTG | TTTT | 205 |
| Human-Exon 52 | 8 | 1 | TATTTCTAAAAGTGTTTTGGCTGG | TTTT | 206 |
| Human-Exon 52 | 9 | 1 | ATTTCTAAAAGTGTTTTGGCTGGT | TTTT | 207 |
| Human-Exon 52 | 10 | 1 | TTTCTAAAAGTGTTTTGGCTGGTC | TTTA | 208 |
| Human-Exon 52 | 11 | 1 | TAAAAGTGTTTTGGCTGGTCTCAC | TTTC | 209 |
| Human-Exon 52 | 12 | −1 | CATAATACAAAGTAAAGTACAATT | TTTA | 210 |
| Human-Exon 52 | 13 | −1 | ACATAATACAAAGTAAAGTACAAT | TTTT | 211 |
| Human-Exon 52 | 14 | 1 | GGCTGGTCTCACAATTGTACTTTA | TTTT | 212 |
| Human-Exon 52 | 15 | 1 | GCTGGTCTCACAATTGTACTTTAC | TTTG | 213 |
| Human-Exon 52 | 16 | 1 | CTTTGTATTATGTAAAAGGAATAC | TTTA | 214 |
| Human-Exon 52 | 17 | 1 | TATTATGTAAAAGGAATACACAAC | TTTG | 215 |
| Human-Exon 52 | 18 | 1 | TTCTTACAGGCAACAATGCAGGAT | TTTG | 216 |
| Human-Exon 52 | 19 | 1 | GAACAGAGGCGTCCCCAGTTGGAA | TTTG | 217 |
| Human-Exon 52 | 20 | −1 | GGCAGCGGTAATGAGTTCTTCCAA | TTTG | 218 |
| Human-Exon 52 | 21 | −1 | TCAAATTTTGGGCAGCGGTAATGA | TTTT | 219 |
| Human-Exon 52 | 22 | 1 | AAAAACAAGACCAGCAATCAAGAG | TTTG | 220 |
| Human-Exon 52 | 23 | −1 | TGTGTCCCATGCTTGTTAAAAAAC | TTTG | 221 |
| Human-Exon 52 | 24 | 1 | TTAACAAGCATGGGACACACAAAG | TTTT | 222 |
| Human-Exon 52 | 25 | 1 | TAACAAGCATGGGACACACAAAGC | TTTT | 223 |
| Human-Exon 52 | 26 | 1 | AACAAGCATGGGACACACAAAGCA | TTTT | 224 |
| Human-Exon 52 | 27 | 1 | ACAAGCATGGGACACACAAAGCAA | TTTA | 225 |
| Human-Exon 52 | 28 | −1 | TTGAAACTTGTCATGCATCTTGCT | TTTA | 226 |
| Human-Exon 52 | 29 | −1 | ATTGAAACTTGTCATGCATCTTGC | TTTT | 227 |
| Human-Exon 52 | 30 | −1 | TATTGAAACTTGTCATGCATCTTG | TTTT | 228 |
| Human-Exon 52 | 31 | 1 | AATAAAAACTTAAGTTCATATATC | TTTC | 229 |
| Human-Exon 50 | 1 | −1 | GTGAATATATTATTGGATTTCTAT | TTTG | 230 |
| Human-Exon 50 | 2 | −1 | AAGATAATTCATGAACATCTTAAT | TTTG | 231 |
| Human-Exon 50 | 3 | −1 | ACAGAAAAGCATACACATTACTTA | TTTA | 232 |
| Human-Exon 50 | 4 | 1 | CTGTTAAAGAGGAAGTTAGAAGAT | TTTT | 233 |
| Human-Exon 50 | 5 | 1 | TGTTAAAGAGGAAGTTAGAAGATC | TTTC | 234 |
| Human-Exon 50 | 6 | −1 | CCGCCTTCCACTCAGAGCTCAGAT | TTTA | 235 |
| Human-Exon 50 | 7 | −1 | CCCTCAGCTCTTGAAGTAAACGGT | TTTG | 236 |
| Human-Exon 50 | 8 | 1 | CTTCAAGAGCTGAGGGCAAAGCAG | TTTA | 237 |
| Human-Exon 50 | 9 | −1 | AACAAATAGCTAGAGCCAAAGAGA | TTTG | 238 |
| Human-Exon 50 | 10 | −1 | GAACAAATAGCTAGAGCCAAAGAG | TTTT | 239 |
| Human-Exon 50 | 11 | 1 | GCTCTAGCTATTTGTTCAAAAGTG | TTTG | 240 |
| Human-Exon 50 | 12 | 1 | TTCAAAAGTGCAACTATGAAGTGA | TTTG | 241 |
| Human-Exon 50 | 13 | −1 | TCTCTCACCCAGTCATCACTTCAT | TTTC | 242 |
| Human-Exon 50 | 14 | −1 | CTCTCTCACCCAGTCATCACTTCA | TTTT | 243 |
| Human-Exon 43 | 1 | 1 | tatatatatatatatTTTTCTCTT | TTTG | 244 |
| Human-Exon 43 | 2 | 1 | TCTCTTTCTATAGACAGCTAATTC | tTTT | 245 |
| Human-Exon 43 | 3 | 1 | CTCTTTCTATAGACAGCTAATTCA | TTTT | 246 |
| Human-Exon 43 | 4 | −1 | AAACAGTAAAAAAATGAATTAGCT | TTTA | 247 |
| Human-Exon 43 | 5 | 1 | TCTTTCTATAGACAGCTAATTCAT | TTTC | 248 |
| Human-Exon 43 | 6 | −1 | AAAACAGTAAAAAAATGAATTAGC | TTTT | 249 |
| Human-Exon 43 | 7 | 1 | TATAGACAGCTAATTCATTTTTTT | TTTC | 250 |
| Human-Exon 43 | 8 | −1 | TATTCTGTAATATAAAAATTTTAA | TTTA | 251 |
| Human-Exon 43 | 9 | −1 | ATATTCTGTAATATAAAAATTTTA | TTTT | 252 |
| Human-Exon 43 | 10 | 1 | TTTACTGTTTTAAAATTTTTATAT | TTTT | 253 |
| Human-Exon 43 | 11 | 1 | TTACTGTTTTAAAATTTTTATATT | TTTT | 254 |
| Human-Exon 43 | 12 | 1 | TACTGTTTTAAAATTTTTATATTA | TTTT | 255 |
| Human-Exon 43 | 13 | 1 | ACTGTTTTAAAATTTTTATATTAC | TTTT | 256 |
| Human-Exon 43 | 14 | 1 | CTGTTTTAAAATTTTTATATTACA | TTTA | 257 |
| Human-Exon 43 | 15 | 1 | AAAATTTTTATATTACAGAATATA | TTTT | 258 |
| Human-Exon 43 | 16 | 1 | AAATTTTTATATTACAGAATATAA | TTTA | 259 |
| Human-Exon 43 | 17 | −1 | TTGTAGACTATCTTTTATATTCTG | TTTG | 260 |
| Human-Exon 43 | 18 | 1 | TATATTACAGAATATAAAAGATAG | TTTT | 261 |
| Human-Exon 43 | 19 | 1 | ATATTACAGAATATAAAAGATAGT | TTTT | 262 |
| Human-Exon 43 | 20 | 1 | TATTACAGAATATAAAAGATAGTC | TTTA | 263 |
| Human-Exon 43 | 21 | −1 | CAATGCTGCTGTCTTCTTGCTATG | TTTG | 264 |
| Human-Exon 43 | 22 | 1 | CAATGGGAAAAAGTTAACAAAATG | TTTC | 265 |
| Human-Exon 43 | 23 | −1 | TGCAAGTATCAAGAAAAATATATG | TTTC | 266 |
| Human-Exon 43 | 24 | 1 | TCTTGATACTTGCAGAAATGATTT | TTTT | 267 |
| Human-Exon 43 | 25 | 1 | CTTGATACTTGCAGAAATGATTTG | TTTT | 268 |
| Human-Exon 43 | 26 | 1 | TTGATACTTGCAGAAATGATTTGT | TTTC | 269 |
| Human-Exon 43 | 27 | 1 | TTTTCAGGGAACTGTAGAATTTAT | TTTG | 270 |
| Human-Exon 43 | 28 | −1 | CATGGAGGGTACTGAAATAAATTC | TTTC | 271 |
| Human-Exon 43 | 29 | −1 | CCATGGAGGGTACTGAAATAAATT | TTTT | 272 |
| Human-Exon 43 | 30 | 1 | CAGGGAACTGTAGAATTTATTTCA | TTTT | 273 |
| Human-Exon 43 | 31 | −1 | TCCATGGAGGGTACTGAAATAAAT | TTTT | 274 |
| Human-Exon 43 | 32 | 1 | AGGGAACTGTAGAATTTATTTCAG | TTTC | 275 |
| Human-Exon 43 | 33 | −1 | TTCCATGGAGGGTACTGAAATAAA | TTTT | 276 |
| Human-Exon 43 | 34 | −1 | CCTGTCTTTTTTCCATGGAGGGTA | TTTC | 277 |
| Human-Exon 43 | 35 | −1 | CCCTGTCTTTTTTCCATGGAGGGT | TTTT | 278 |
| Human-Exon 43 | 36 | −1 | TCCCTGTCTTTTTTCCATGGAGGG | TTTT | 279 |
| Human-Exon 43 | 37 | 1 | TTTCAGTACCCTCCATGGAAAAAA | TTTA | 280 |
| Human-Exon 43 | 38 | 1 | AGTACCCTCCATGGAAAAAAGACA | TTTC | 281 |
| Human-Exon 6 | 1 | 1 | AGTTTGCATGGTTCTTGCTCAAGG | TTTA | 282 |
| Human-Exon 6 | 2 | −1 | ATAAGAAAATGCATTCCTTGAGCA | TTTC | 283 |
| Human-Exon 6 | 3 | −1 | CATAAGAAAATGCATTCCTTGAGC | TTTT | 284 |
| Human-Exon 6 | 4 | 1 | CATGGTTCTTGCTCAAGGAATGCA | TTTG | 285 |
| Human-Exon 6 | 5 | −1 | ACCTACATGTGGAAATAAATTTTC | TTTG | 286 |
| Human-Exon 6 | 6 | −1 | GACCTACATGTGGAAATAAATTTT | TTTT | 287 |
| Human-Exon 6 | 7 | −1 | TGACCTACATGTGGAAATAAATTT | TTTT | 288 |
| Human-Exon 6 | 8 | 1 | CTTATGAAAATTTATTTCCACATG | TTTT | 289 |
| Human-Exon 6 | 9 | 1 | TTATGAAAATTTATTTCCACATGT | TTTC | 290 |
| Human-Exon 6 | 10 | −1 | ATTACATTTTTGACCTACATGTGG | TTTC | 291 |
| Human-Exon 6 | 11 | −1 | CATTACATTTTTGACCTACATGTG | TTTT | 292 |
| Human-Exon 6 | 12 | −1 | TCATTACATTTTTGACCTACATGT | TTTT | 293 |
| Human-Exon 6 | 13 | 1 | TTTCCACATGTAGGTCAAAAATGT | TTTA | 294 |
| Human-Exon 6 | 14 | 1 | CACATGTAGGTCAAAAATGTAATG | TTTC | 295 |
| Human-Exon 6 | 15 | −1 | TTGCAATCCAGCCATGATATTTTT | TTTG | 296 |
| Human-Exon 6 | 16 | −1 | ACTGTTGGTTTGTTGCAATCCAGC | TTTC | 297 |
| Human-Exon 6 | 17 | −1 | CACTGTTGGTTTGTTGCAATCCAG | TTTT | 298 |
| Human-Exon 6 | 18 | 1 | AATGCTCTCATCCATAGTCATAGG | TTTG | 299 |
| Human-Exon 6 | 19 | −1 | ATGTCTCAGTAATCTTCTTACCTA | TTTA | 300 |
| Human-Exon 6 | 20 | −1 | CAAGTTATTTAATGTCTCAGTAAT | TTTA | 301 |
| Human-Exon 6 | 21 | −1 | ACAAGTTATTTAATGTCTCAGTAA | TTTT | 302 |
| Human-Exon 6 | 22 | 1 | GACTCTGATGACATATTTTTCCCC | TTTA | 303 |
| Human-Exon 6 | 23 | 1 | TCCCCAGTATGGTTCCAGATCATG | TTTT | 304 |
| Human-Exon 6 | 24 | 1 | CCCCAGTATGGTTCCAGATCATGT | TTTT | 305 |
| Human-Exon 6 | 25 | 1 | CCCAGTATGGTTCCAGATCATGTC | TTTC | 306 |
| Human-Exon 7 | 1 | 1 | TATTTGTCTTtgtgtatgtgtgta | TTTA | 307 |
| Human-Exon 7 | 2 | 1 | TCTTtgtgtatgtgtgtatgtgta | TTTG | 308 |
| Human-Exon 7 | 3 | 1 | tgtatgtgtgtatgtgtatgtgtt | TTtg | 309 |
| Human-Exon 7 | 4 | 1 | AGGCCAGACCTATTTGACTGGAAT | ttTT | 310 |
| Human-Exon 7 | 5 | 1 | GGCCAGACCTATTTGACTGGAATA | tTTA | 311 |
| Human-Exon 7 | 6 | 1 | ACTGGAATAGTGTGGTTTGCCAGC | TTTG | 312 |
| Human-Exon 7 | 7 | 1 | CCAGCAGTCAGCCACACAACGACT | TTTG | 313 |
| Human-Exon 7 | 8 | −1 | TCTATGCCTAATTGATATCTGGCG | TTTC | 314 |
| Human-Exon 7 | 9 | −1 | CCAACCTTCAGGATCGAGTAGTTT | TTTA | 315 |
| Human-Exon 7 | 10 | 1 | TGGACTACCACTGCTTTTAGTATG | TTTC | 316 |
| Human-Exon 7 | 11 | 1 | AGTATGGTAGAGTTTAATGTTTTC | TTTT | 317 |
| Human-Exon 7 | 12 | 1 | GTATGGTAGAGTTTAATGTTTTCA | TTTA | 318 |
| Human-Exon 8 | 1 | −1 | AGACTCTAAAAGGATAATGAACAA | TTTG | 319 |
| Human-Exon 8 | 2 | 1 | ACTTTGATTTGTTCATTATCCTTT | TTTA | 320 |
| Human-Exon 8 | 3 | −1 | TATATTTGAGACTCTAAAAGGATA | TTTC | 321 |
| Human-Exon 8 | 4 | 1 | ATTTGTTCATTATCCTTTTAGAGT | TTTG | 322 |
| Human-Exon 8 | 5 | −1 | GTTTCTATATTTGAGACTCTAAAA | TTTG | 323 |
| Human-Exon 8 | 6 | −1 | GGTTTCTATATTTGAGACTCTAAA | TTTT | 324 |
| Human-Exon 8 | 7 | −1 | TGGTTTCTATATTTGAGACTCTAA | TTTT | 325 |
| Human-Exon 8 | 8 | 1 | TTCATTATCCTTTTAGAGTCTCAA | TTTG | 326 |
| Human-Exon 8 | 9 | 1 | AGAGTCTCAAATATAGAAACCAAA | TTTT | 327 |
| Human-Exon 8 | 10 | 1 | GAGTCTCAAATATAGAAACCAAAA | TTTA | 328 |
| Human-Exon 8 | 11 | −1 | CACTTCCTGGATGGCTTCAATGCT | TTTC | 329 |
| Human-Exon 8 | 12 | 1 | GCCTCAACAAGTGAGCATTGAAGC | TTTT | 330 |
| Human-Exon 8 | 13 | 1 | CCTCAACAAGTGAGCATTGAAGCC | TTTG | 331 |
| Human-Exon 8 | 14 | −1 | GGTGGCCTTGGCAACATTTCCACT | TTTA | 332 |
| Human-Exon 8 | 15 | −1 | GTCACTTTAGGTGGCCTTGGCAAC | TTTA | 333 |
| Human-Exon 8 | 16 | −1 | ATGATGTAACTGAAAATGTTCTTC | TTTG | 334 |
| Human-Exon 8 | 17 | −1 | CCTGTTGAGAATAGTGCATTTGAT | TTTA | 335 |
| Human-Exon 8 | 18 | 1 | CAGTTACATCATCAAATGCACTAT | TTTT | 336 |
| Human-Exon 8 | 19 | 1 | AGTTACATCATCAAATGCACTATT | TTTC | 337 |
| Human-Exon 8 | 20 | −1 | CACACTTTACCTGTTGAGAATAGT | TTTA | 338 |
| Human-Exon 8 | 21 | 1 | CTGTTTTATATGCATTTTTAGGTA | TTTT | 339 |
| Human-Exon 8 | 22 | 1 | TGTTTTATATGCATTTTTAGGTAT | TTTC | 340 |
| Human-Exon 8 | 23 | 1 | ATATGCATTTTTAGGTATTACGTG | TTTT | 341 |
| Human-Exon 8 | 24 | 1 | TATGCATTTTTAGGTATTACGTGC | TTTA | 342 |
| Human-Exon 8 | 25 | 1 | TAGGTATTACGTGCACatatatat | TTTT | 343 |
| Human-Exon 8 | 26 | 1 | AGGTATTACGTGCACatatatata | TTTT | 344 |
| Human-Exon 8 | 27 | 1 | GGTATTACGTGCACatatatatat | TTTA | 345 |
| Human-Exon 55 | 1 | −1 | AGCAACAACTATAATATTGTGCAG | TTTA | 346 |
| Human-Exon 55 | 2 | 1 | GTTCCTCCATCTTTCTCTTTTTAT | TTTA | 347 |
| Human-Exon 55 | 3 | 1 | TCTTTTTATGGAGTTCACTAGGTG | TTTC | 348 |
| Human-Exon 55 | 4 | 1 | TATGGAGTTCACTAGGTGCACCAT | TTTT | 349 |
| Human-Exon 55 | 5 | 1 | ATGGAGTTCACTAGGTGCACCATT | TTTT | 350 |
| Human-Exon 55 | 6 | 1 | TGGAGTTCACTAGGTGCACCATTC | TTTA | 351 |
| Human-Exon 55 | 7 | 1 | ATAATTGCATCTGAACATTTGGTC | TTTA | 352 |
| Human-Exon 55 | 8 | 1 | GTCCTTTGCAGGGTGAGTGAGCGA | TTTG | 353 |
| Human-Exon 55 | 9 | −1 | TTCCAAAGCAGCCTCTCGCTCACT | TTTC | 354 |
| Human-Exon 55 | 10 | 1 | CAGGGTGAGTGAGCGAGAGGCTGC | TTTG | 355 |
| Human-Exon 55 | 11 | 1 | GAAGAAACTCATAGATTACTGCAA | TTTG | 356 |
| Human-Exon 55 | 12 | −1 | CAGGTCCAGGGGGAACTGTTGCAG | TTTC | 357 |
| Human-Exon 55 | 13 | −1 | CCAGGTCCAGGGGGAACTGTTGCA | TTTT | 358 |
| Human-Exon 55 | 14 | −1 | AGCTTCTGTAAGCCAGGCAAGAAA | TTTC | 359 |
| Human-Exon 55 | 15 | 1 | TTGCCTGGCTTACAGAAGCTGAAA | TTTC | 360 |
| Human-Exon 55 | 16 | −1 | CTTACGGGTAGCATCCTGTAGGAC | TTTC | 361 |
| Human-Exon 55 | 17 | −1 | CTCCCTTGGAGTCTTCTAGGAGCC | TTTA | 362 |
| Human-Exon 55 | 18 | −1 | ACTCCCTTGGAGTCTTCTAGGAGC | TTTT | 363 |
| Human-Exon 55 | 19 | −1 | ATCAGCTCTTTTACTCCCTTGGAG | TTTC | 364 |
| Human-Exon 55 | 20 | 1 | CGCTTTAGCACTCTTGTGGATCCA | TTTC | 365 |
| Human-Exon 55 | 21 | 1 | GCACTCTTGTGGATCCAATTGAAC | TTTA | 366 |
| Human-Exon 55 | 22 | −1 | TCCCTGGCTTGTCAGTTACAAGTA | TTTG | 367 |
| Human-Exon 55 | 23 | −1 | GTCCCTGGCTTGTCAGTTACAAGT | TTTT | 368 |
| Human-Exon 55 | 24 | −1 | TTTTGTCCCTGGCTTGTCAGTTAC | TTTG | 369 |
| Human-Exon 55 | 25 | −1 | GTTTTGTCCCTGGCTTGTCAGTTA | TTTT | 370 |
| Human-Exon 55 | 26 | 1 | TACTTGTAACTGACAAGCCAGGGA | TTTG | 371 |
| Human-G1-exon51 | | 1 | gCTCCTACTCAGACTGTTACTCTG | TTTA | 372 |
| Human-G2-exon51 | | 1 | taccatgtattgctaaacaaagta | TTTC | 373 |
| Human-G3-exon51 | | −1 | attgaagagtaacaatttgagcca | TTTA | 374 |
| mouse-Exon23-G1 | | 1 | aggctctgcaaagttctTTGAAAG | TTTG | 375 |
| mouse-Exon23-G2 | | 1 | AAAGAGCAACAAAATGGCttcaac | TTTG | 376 |
| mouse-Exon23-G3 | | 1 | AAAGAGCAATAAAATGGCttcaac | TTTG | 377 |
| mouse-Exon23-G4 | | −1 | AAAGAACTTTGCAGAGCctcaaaa | TTTC | 378 |
| mouse-Exon23-G5 | | −1 | ctgaatatctatgcattaataact | TTTA | 379 |
| mouse-Exon23-G6 | | −1 | tattatattacagggcatattata | TTTC | 380 |
| mouse-Exon23-G7 | | 1 | Aggtaagccgaggtttggccttta | TTTC | 381 |
| mouse-Exon23-G8 | | 1 | cccagagtccttcaaagatattga | TTTA | 382 |

*In this table, upper case letters represent nucleotides that align to the exon
sequence of the gene. Lower case letters represent nucleotides that align to the
intron sequence of the gene.

TABLE E
gRNA sequences

| Tar- | | | | | |
|---|---|---|---|---|---|
| geted | | | | | SEQ |
| gRNA | Guide | | | | ID |
| Exon | # | Strand | gRNA sequence* | PAM | NO. |
| Human- | 4 | 1 | aaaaaggaaaaaagaagaaaaaga | tttt | 448 |
| Exon | | | | | |
| 51 | | | | | |
| Human- | 5 | 1 | Caaaaaggaaaaaagaagaaaaag | tttt | 449 |
| Exon | | | | | |
| 51 | | | | | |
| Human- | 6 | 1 | GCaaaaaggaaaaaagaagaaaaa | tttc | 450 |
| Exon | | | | | |
| 51 | | | | | |
| Human- | 7 | 1 | UUUUGCaaaaaggaaaaaagaaga | tttt | 451 |
| Exon | | | | | |
| 51 | | | | | |
| Human- | 8 | 1 | UUUUUGCaaaaaggaaaaaagaag | tttt | 452 |
| Exon | | | | | |
| 51 | | | | | |
| Human- | 9 | 1 | GUUUUUGCaaaaaggaaaaaagaa | tttc | 453 |
| Exon | | | | | |
| 51 | | | | | |
| Human- | 10 | 1 | AUUUUGGGUUUUUGCaaaaaggaa | tttt | 454 |
| Exon | | | | | |
| 51 | | | | | |
| Human- | 11 | 1 | UAUUUUGGGUUUUUGCaaaaagga | tttt | 455 |
| Exon | | | | | |
| 51 | | | | | |
| Human- | 12 | 1 | AUAUUUUGGGUUUUUGCaaaaagg | tttt | 456 |
| Exon | | | | | |
| 51 | | | | | |
| Human- | 13 | 1 | AAUAUUUUGGGUUUUUGCaaaaag | tttc | 457 |
| Exon | | | | | |
| 51 | | | | | |
| Human- | 14 | 1 | GCUAAAAUAUUUUGGGUUUUUGCa | tttt | 458 |
| Exon | | | | | |
| 51 | | | | | |
| Human- | 15 | 1 | AGCUAAAAUAUUUUGGGUUUUUGC | tttt | 459 |
| Exon | | | | | |
| 51 | | | | | |
| Human- | 16 | 1 | GAGCUAAAAUAUUUUGGGUUUUUG | tttG | 460 |
| Exon | | | | | |
| 51 | | | | | |
| Human- | 17 | 1 | AGAGUAACAGUCUGAGUAGGAGCU | TTTT | 461 |
| Exon | | | | | |
| 51 | | | | | |
| Human- | 18 | 1 | CAGAGUAACAGUCUGAGUAGGAGC | TTTA | 462 |
| Exon | | | | | |
| 51 | | | | | |
| Human- | 19 | −1 | GUGACACAACCUGUGGUUACUAAG | TTTC | 463 |
| Exon | | | | | |
| 51 | | | | | |
| Human- | 20 | −1 | GGUUACUAAGGAAACUGCCAUCU | TTTG | 464 |
| Exon | | | | | |
| 51 | | | | | |
| Human- | 21 | −1 | AAGGAAACUGCCAUCUCCAAACUA | TTTC | 465 |
| Exon | | | | | |
| 51 | | | | | |
| Human- | 22 | −1 | AUCAUCAAGCAGAAGGUAUGAGAA | TTTT | 466 |
| Exon | | | | | |
| 51 | | | | | |
| Human- | 23 | −1 | AGCAGAAGGUAUGAGAAAAAAUGA | TTTA | 467 |
| Exon | | | | | |
| 51 | | | | | |
| Human- | 24 | −1 | GCAGAAGGUAUGAGAAAAAAUGAU | TTTT | 468 |
| Exon | | | | | |
| 51 | | | | | |
| Human- | 25 | −1 | UAAAAGUUGGCAGAAGUUUUUCUU | TTTA | 469 |
| Exon | | | | | |
| 51 | | | | | |
| Human- | 26 | −1 | AAAAGUUGGCAGAAGUUUUUCUUU | TTTT | 470 |
| Exon | | | | | |
| 51 | | | | | |
| Human- | 27 | 1 | GGUGGAAAAUCUUCAUUUUAAAGA | TTTT | 471 |
| Exon | | | | | |
| 51 | | | | | |
| Human- | 28 | 1 | UGGUGGAAAAUCUUCAUUUUAAAG | TTTT | 472 |
| Exon | | | | | |
| 51 | | | | | |
| Human- | 29 | 1 | UUGGUGGAAAAUCUUCAUUUUAAA | TTTC | 473 |
| Exon | | | | | |
| 51 | | | | | |
| Human- | 30 | 1 | GUGAUUGGUGGAAAAUCUUCAUUU | TTTA | 474 |
| Exon | | | | | |
| 51 | | | | | |
| Human- | 31 | 1 | CUAGGAGAGUAAAGUGAUUGGUGG | TTTT | 475 |
| Exon | | | | | |
| 51 | | | | | |
| Human- | 32 | 1 | UCUAGGAGAGUAAAGUGAUUGGUG | TTTC | 476 |
| Exon | | | | | |
| 51 | | | | | |
| Human- | 33 | 1 | CUGGUGGGAAAUGGUCUAGGAGA | TTTA | 477 |
| Exon | | | | | |
| 51 | | | | | |
| Human- | 1 | −1 | guagcacacuguuuaaucuuuucu | tttg | 478 |
| Exon | | | | | |
| 45 | | | | | |
| Human- | 2 | −1 | cacacuguuuaaucuuuucucaaa | TTTa | 479 |
| Exon | | | | | |
| 45 | | | | | |
| Human- | 3 | −1 | acacuguuuaaucuuuucucaaau | TTTT | 480 |
| Exon | | | | | |
| 45 | | | | | |
| Human- | 4 | −1 | cacuguuuaaucuuuucucaaauA | TTTT | 481 |
| Exon | | | | | |
| 45 | | | | | |
| Human- | 5 | 1 | AUGUCUUUUUauuugagaaaagau | ttta | 482 |
| Exon | | | | | |
| 45 | | | | | |
| Human- | 6 | 1 | AAGCCCCAUGUCUUUUUauuugag | tttt | 483 |
| Exon | | | | | |
| 45 | | | | | |
| Human- | 7 | 1 | GAAGCCCCAUGUCUUUUUauuuga | tttc | 484 |
| Exon | | | | | |
| 45 | | | | | |
| Human- | 8 | 1 | GUAAGAUACCAAAAAGGCAAAACA | TTTT | 485 |
| Exon | | | | | |
| 45 | | | | | |
| Human- | 9 | 1 | UGUAAGAUACCAAAAAGGCAAAAC | TTTT | 486 |
| Exon | | | | | |
| 45 | | | | | |
| Human- | 10 | 1 | CUGUAAGAUACCAAAAAGGCAAAA | TTTG | 487 |
| Exon | | | | | |
| 45 | | | | | |
| Human- | 11 | 1 | GUUCCUGUAAGAUACCAAAAAGGC | TTTT | 488 |
| Exon | | | | | |
| 45 | | | | | |
| Human- | 12 | 1 | AGUUCCUGUAAGAUACCAAAAAGG | TTTG | 489 |
| Exon | | | | | |
| 45 | | | | | |
| Human- | 13 | 1 | UCCUGGAGUUCCUGUAAGAUACCA | TTTT | 490 |
| Exon | | | | | |
| 45 | | | | | |
| Human- | 14 | 1 | AUCCUGGAGUUCCUGUAAGAUACC | TTTT | 491 |
| Exon | | | | | |
| 45 | | | | | |
| Human- | 15 | −1 | GGGAAGAAAUAAUUCAGCAAUCCU | TTTG | 492 |
| Exon | | | | | |
| 45 | | | | | |
| Human- | 16 | −1 | GGAAGAAAUAAUUCAGCAAUCCUC | TTTT | 493 |
| Exon | | | | | |
| 45 | | | | | |
| Human- | 17 | −1 | GAAGAAAUAAUUCAGCAAUCCUCA | TTTT | 494 |
| Exon | | | | | |
| 45 | | | | | |
| Human- | 18 | −1 | AAAACAGAUGCCAGUAUUCUACAG | TTTC | 495 |
| Exon | | | | | |
| 45 | | | | | |
| Human- | 19 | −1 | AAACAGAUGCCAGUAUUCUACAGG | TTTT | 496 |
| Exon | | | | | |
| 45 | | | | | |
| Human- | 20 | −1 | AACAGAUGCCAGUAUUCUACAGGA | TTTT | 497 |
| Exon | | | | | |
| 45 | | | | | |
| Human- | 21 | −1 | GAAUCUGCGGUGGCAGGAGGUCUG | TTTG | 498 |
| Exon | | | | | |
| 45 | | | | | |
| Human- | 22 | −1 | AGGUCUGCAAACAGCUGUCAGACA | TTTC | 499 |
| Exon | | | | | |
| 45 | | | | | |
| Human- | 23 | −1 | GGUCUGCAAACAGCUGUCAGACAG | TTTT | 500 |
| Exon | | | | | |
| 45 | | | | | |
| Human- | 24 | −1 | GUCUGCAAACAGCUGUCAGACAGA | TTTT | 501 |
| Exon | | | | | |
| 45 | | | | | |
| Human- | 25 | −1 | UCUGCAAACAGCUGUCAGACAGAA | TTTT | 502 |
| Exon | | | | | |
| 45 | | | | | |
| Human- | 26 | −1 | UAGGGCGACAGAUCUAAUAGGAAU | TTTC | 503 |
| Exon | | | | | |
| 45 | | | | | |
| Human- | 27 | −1 | AGGGCGACAGAUCUAAUAGGAAUG | TTTT | 504 |
| Exon | | | | | |
| 45 | | | | | |
| Human- | 28 | 1 | UAAAGAAAGCUUAAAAAGUCUGCU | TTTT | 505 |
| Exon | | | | | |
| 45 | | | | | |
| Human- | 29 | 1 | CUAAAGAAAGCUUAAAAAGUCUGC | TTTA | 506 |
| Exon | | | | | |
| 45 | | | | | |
| Human- | 30 | 1 | AAAUAUUCUUCUAAAGAAAGCUUA | TTTT | 507 |
| Exon | | | | | |
| 45 | | | | | |
| Human- | 31 | 1 | GAAAUAUUCUUCUAAAGAAAGCUU | TTTT | 508 |
| Exon | | | | | |
| 45 | | | | | |
| Human- | 32 | 1 | UGAAAUAUUCUUCUAAAGAAAGCU | TTTA | 509 |
| Exon | | | | | |
| 45 | | | | | |
| Human- | 33 | 1 | UCUCUCAUGAAAUAUUCUUCUAAA | TTTC | 510 |
| Exon | | | | | |
| 45 | | | | | |
| Human- | 34 | 1 | AUAAUCUCUCAUGAAAUAUUCUUC | TTTA | 511 |
| Exon | | | | | |
| 45 | | | | | |
| Human- | 1 | 1 | GCGUAUAUUUUUUGGUUAUACUGA | TTTG | 512 |
| Exon | | | | | |
| 44 | | | | | |
| Human- | 2 | 1 | ucaagaaaaauagauggauuaugu | tttt | 513 |
| Exon | | | | | |
| 44 | | | | | |
| Human- | 3 | 1 | aucaagaaaaauagauggauuaug | ttta | 514 |
| Exon | | | | | |
| 44 | | | | | |
| Human- | 4 | 1 | CAGGUaaaagcauauggaucaaga | tttt | 515 |
| Exon | | | | | |
| 44 | | | | | |
| Human- | 5 | 1 | GCAGGUaaaagcauauggaucaag | tttt | 516 |
| Exon | | | | | |
| 44 | | | | | |
| Human- | 6 | 1 | UGCAGGUaaaagcauauggaucaa | tttc | 517 |
| Exon | | | | | |
| 44 | | | | | |
| Human- | 7 | −1 | CAGGCGAUUUGACAGAUCUGUUGA | TTTC | 518 |
| Exon | | | | | |
| 44 | | | | | |
| Human- | 8 | 1 | AGAUCUGUCAAAUCGCCUGCAGGU | tttt | 519 |
| Exon | | | | | |
| 44 | | | | | |
| Human- | 9 | 1 | CAGAUCUGUCAAAUCGCCUGCAGG | tttA | 520 |
| Exon | | | | | |
| 44 | | | | | |
| Human- | 10 | 1 | GCCGCCAUUUCUCAACAGAUCUGU | TTTG | 521 |
| Exon | | | | | |
| 44 | | | | | |
| Human- | 11 | −1 | AAUGGCGGCGUUUUCAUUAUGAUA | TTTA | 522 |
| Exon | | | | | |
| 44 | | | | | |
| Human- | 12 | 1 | AUUAAAUAUCUUUAUAUCAUAAUG | TTTT | 523 |
| Exon | | | | | |
| 44 | | | | | |
| Human- | 13 | −1 | UGAGAAUUGGGAACAUGCUAAAUA | TTTG | 524 |
| Exon | | | | | |
| 44 | | | | | |
| Human- | 14 | −1 | GGUAAGUCUUUGAUUUGUUUUUUC | TTTC | 525 |
| Exon | | | | | |
| 44 | | | | | |
| Human- | 15 | 1 | AAAUACAAUUUCGAAAAAACAAAU | TTTG | 526 |
| Exon | | | | | |
| 44 | | | | | |
| Human- | 16 | 1 | AAGAUAAAUACAAUUUCGAAAAAA | TTTG | 527 |
| Exon | | | | | |
| 44 | | | | | |
| Human- | 17 | 1 | GCUGAAGAUAAAUACAAUUUCGAA | TTTT | 528 |
| Exon | | | | | |
| 44 | | | | | |
| Human- | 18 | 1 | UGCUGAAGAUAAAUACAAUUUCGA | TTTT | 529 |
| Exon | | | | | |
| 44 | | | | | |
| Human- | 19 | 1 | GUGCUGAAGAUAAAUACAAUUUCG | TTTT | 530 |
| Exon | | | | | |
| 44 | | | | | |
| Human- | 20 | 1 | UGUGCUGAAGAUAAAUACAAUUUC | TTTC | 531 |
| Exon | | | | | |
| 44 | | | | | |
| Human- | 21 | −1 | GCACAUCUGGACUCUUUAACUUCU | TTTA | 532 |
| Exon | | | | | |
| 44 | | | | | |
| Human- | 22 | 1 | UAAAGAGUCCAGAUGUGCUGAAGA | TTTA | 533 |
| Exon | | | | | |
| 44 | | | | | |
| Human- | 23 | −1 | AAGAUCAGGUUCUGAAGGGUGAUG | TTTC | 534 |
| Exon | | | | | |
| 44 | | | | | |
| Human- | 24 | 1 | UUCAGAACCUGAUCUUUAAGAAGU | TTTA | 535 |
| Exon | | | | | |
| 44 | | | | | |
| Human- | 25 | 1 | AAUAUAAUGAUGACAACAACAGUC | TTTT | 536 |
| Exon | | | | | |
| 44 | | | | | |
| Human- | 26 | 1 | UAAUAUAAUGAUGACAACAACAGU | TTTG | 537 |
| Exon | | | | | |
| 44 | | | | | |
| Human- | 1 | −1 | UUUAUUUUUCCUUUUAUUCUAGUU | TTTC | 538 |
| Exon | | | | | |
| 53 | | | | | |
| Human- | 2 | 1 | AAAGGAAAAAUAAAUAUAUAGUAG | TTTA | 539 |
| Exon | | | | | |
| 53 | | | | | |
| Human- | 3 | 1 | UUUCAACUAGAAUAAAAGGAAAAA | TTTA | 540 |
| Exon | | | | | |
| 53 | | | | | |
| Human- | 4 | 1 | AUUCUUUCAACUAGAAUAAAAGGA | TTTT | 541 |
| Exon | | | | | |
| 53 | | | | | |
| Human- | 5 | 1 | AAUUCUUUCAACUAGAAUAAAAGG | TTTT | 542 |
| Exon | | | | | |
| 53 | | | | | |
| Human- | 6 | 1 | GAAUUCUUUCAACUAGAAUAAAAG | TTTC | 543 |
| Exon | | | | | |
| 53 | | | | | |
| Human- | 7 | 1 | AUUCUGAAUUCUUUCAACUAGAAU | TTTT | 544 |
| Exon | | | | | |
| 53 | | | | | |
| Human- | 8 | 1 | GAUUCUGAAUUCUUUCAACUAGAA | TTTA | 545 |
| Exon | | | | | |
| 53 | | | | | |
| Human- | 9 | −1 | CAGAACCGGAGGCAACAGUUGAAU | TTTC | 546 |
| Exon | | | | | |
| 53 | | | | | |
| Human- | 10 | −1 | GGAGGCAACAGUUGAAUGAAAUGU | TTTA | 547 |
| Exon | | | | | |
| 53 | | | | | |
| Human- | 11 | −1 | UAUACAGUAGAUGCAAUCCAAAAG | TTTT | 548 |
| Exon | | | | | |
| 53 | | | | | |
| Human- | 12 | −1 | GAUGCAAUCCAAAAGAAAAUCACA | TTTC | 549 |
| Exon | | | | | |
| 53 | | | | | |
| Human- | 13 | −1 | AAUCACAGAAACCAAGGUUAGUAU | TTTG | 550 |
| Exon | | | | | |
| 53 | | | | | |
| Human- | 14 | −1 | AGGUUAGUAUCAAAGAUACCUUU | TTTA | 551 |
| Exon | | | | | |
| 53 | | | | | |
| Human- | 15 | −1 | GGUUAGUAUCAAAGAUACCUUUUU | TTTT | 552 |
| Exon | | | | | |
| 53 | | | | | |
| Human- | 16 | −1 | AGUAUCAAAGAUACCUUUUUAAAA | TTTA | 553 |
| Exon | | | | | |
| 53 | | | | | |
| Human- | 17 | −1 | GUAUCAAAGAUACCUUUUUAAAAU | TTTT | 554 |
| Exon | | | | | |
| 53 | | | | | |
| Human- | 1 | −1 | UGUUUGUGUCCCAGUUUGCAUUAA | TTTG | 555 |
| Exon | | | | | |
| 46 | | | | | |
| Human- | 2 | 1 | CUGGGACACAAACAUGGCAAUUUA | TTTT | 556 |
| Exon | | | | | |
| 46 | | | | | |
| Human- | 3 | 1 | ACUGGGACACAAACAUGGCAAUUU | TTTT | 557 |
| Exon | | | | | |
| 46 | | | | | |
| Human- | 4 | 1 | AACUGGGACACAAACAUGGCAAUU | TTTA | 558 |
| Exon | | | | | |
| 46 | | | | | |
| Human- | 5 | 1 | UAUUUGUUAAUGCAAACUGGGACA | TTTG | 559 |
| Exon | | | | | |
| 46 | | | | | |
| Human- | 6 | −1 | ACAAAUAGUUUGAGAACUAUGUUG | tttC | 560 |
| Exon | | | | | |
| 46 | | | | | |
| Human- | 7 | −1 | CAAAUAGUUUGAGAACUAUGUUGG | tttt | 561 |
| Exon | | | | | |
| 46 | | | | | |
| Human- | 8 | −1 | AAAUAGUUUGAGAACUAUGUUGGa | tttt | 562 |
| Exon | | | | | |
| 46 | | | | | |
| Human- | 9 | −1 | AUAGUUUGAGAACUAUGUUGGaaa | tttt | 563 |
| Exon | | | | | |
| 46 | | | | | |
| Human- | 10 | −1 | UAGUUUGAGAACUAUGUUGGaaaa | tttt | 564 |
| Exon | | | | | |
| 46 | | | | | |
| Human- | 11 | −1 | AGUUUGAGAACUAUGUUGGaaaaa | tttt | 565 |
| Exon | | | | | |
| 46 | | | | | |
| Human- | 12 | 1 | UAGUUCUCAAACUAUUUGUUAAUG | TTTG | 566 |
| Exon | | | | | |
| 46 | | | | | |
| Human- | 13 | 1 | UAuuuuuuuuuCCAACAUAGUUCU | TTTG | 567 |
| Exon | | | | | |
| 46 | | | | | |
| Human- | 14 | −1 | CUUCUUUCUCCAGGCUAGAAGAAC | TTTT | 568 |
| Exon | | | | | |
| 46 | | | | | |
| Human- | 15 | 1 | CUUCUAGCCUGGAGAAAGAAGAAU | TTTT | 569 |
| Exon | | | | | |
| 46 | | | | | |
| Human- | 16 | 1 | UCUUCUAGCCUGGAGAAAGAAGAA | TTTA | 570 |
| Exon | | | | | |
| 46 | | | | | |
| Human- | 17 | 1 | AUUCUUUUGUUCUUCUAGCCUGGA | TTTC | 571 |
| Exon | | | | | |
| 46 | | | | | |
| Human- | 18 | −1 | CAAAAGAAUAUCUUGUCAGAAUUU | TTTG | 572 |
| Exon | | | | | |
| 46 | | | | | |
| Human- | 19 | −1 | CUGGAAAAGAGCAGCAACUAAAAG | TTTT | 573 |
| Exon | | | | | |
| 46 | | | | | |
| Human- | 20 | −1 | CAAGUCAAGGUAAUUUUAUUUUCU | TTTG | 574 |
| Exon | | | | | |
| 46 | | | | | |
| Human- | 21 | −1 | CAAAUCCCCCAGGGCCUGCUUGCA | TTTA | 575 |
| Exon | | | | | |
| 46 | | | | | |
| Human- | 22 | 1 | AGGCCCUGGGGGAUUUGAGAAAAU | TTTT | 576 |
| Exon | | | | | |
| 46 | | | | | |
| Human- | 23 | 1 | CAGGCCCUGGGGGAUUUGAGAAAA | TTTA | 577 |
| Exon | | | | | |
| 46 | | | | | |
| Human- | 24 | 1 | CAAGCAGGCCCUGGGGGAUUUGAG | TTTT | 578 |
| Exon | | | | | |
| 46 | | | | | |
| Human- | 25 | 1 | GCAAGCAGGCCCUGGGGGAUUUGA | TTTC | 579 |
| Exon | | | | | |
| 46 | | | | | |
| Human- | 26 | 1 | GCAGAAAACCAAUGAUUGAAUUAA | TTTT | 580 |
| Exon | | | | | |
| 46 | | | | | |
| Human- | 27 | 1 | GGCAGAAAACCAAUGAUUGAAUUA | TTTT | 581 |
| Exon | | | | | |
| 46 | | | | | |
| Human- | 28 | 1 | GGGCAGAAAACCAAUGAUUGAAUU | TTTT | 582 |
| Exon | | | | | |
| 46 | | | | | |
| Human- | 29 | 1 | UGGGCAGAAAACCAAUGAUUGAAU | TTTA | 583 |
| Exon | | | | | |
| 46 | | | | | |
| Human- | 30 | −1 | AUUAGGUUAUUCAUAGUUCCUUGC | TTTA | 584 |
| Exon | | | | | |
| 46 | | | | | |
| Human- | 31 | 1 | AACUAUGAAUAACCUAAUGGGCAG | TTTT | 585 |
| Exon | | | | | |
| 46 | | | | | |
| Human- | 32 | 1 | GAACUAUGAAUAACCUAAUGGGCA | TTTC | 586 |
| Exon | | | | | |
| 46 | | | | | |
| Human- | 1 | −1 | UAUUUCCUGUUAAAUUGUUUUCUA | TTTA | 587 |
| Exon | | | | | |
| 52 | | | | | |
| Human- | 2 | 1 | GGUUUAUAGAAAACAAUUUAACAG | TTTC | 588 |
| Exon | | | | | |
| 52 | | | | | |
| Human- | 3 | −1 | AUACAGUAACAUCUUUUUUAUUUC | TTTA | 589 |
| Exon | | | | | |
| 52 | | | | | |
| Human- | 4 | −1 | UACAGUAACAUCUUUUUUAUUUCU | TTTT | 590 |
| Exon | | | | | |
| 52 | | | | | |
| Human- | 5 | 1 | AUGUUACUGUAUAAGGGUUUAUAG | TTTT | 591 |
| Exon | | | | | |
| 52 | | | | | |
| Human- | 6 | 1 | GAUGUUACUGUAUAAGGGUUUAUA | TTTC | 592 |
| Exon | | | | | |
| 52 | | | | | |
| Human- | 7 | 1 | CAGCCAAAACACUUUUAGAAAUAA | TTTT | 593 |
| Exon | | | | | |
| 52 | | | | | |
| Human- | 8 | 1 | CCAGCCAAAACACUUUUAGAAAUA | TTTT | 594 |
| Exon | | | | | |
| 52 | | | | | |
| Human- | 9 | 1 | ACCAGCCAAAACACUUUUAGAAAU | TTTT | 595 |
| Exon | | | | | |
| 52 | | | | | |
| Human- | 10 | 1 | GACCAGCCAAAACACUUUUAGAAA | TTTA | 596 |
| Exon | | | | | |
| 52 | | | | | |
| Human- | 11 | 1 | GUGAGACCAGCCAAAACACUUUUA | TTTC | 597 |
| Exon | | | | | |
| 52 | | | | | |
| Human- | 12 | −1 | AAUUGUACUUUACUUUGUAUUAUG | TTTA | 598 |
| Exon | | | | | |
| 52 | | | | | |
| Human- | 13 | −1 | AUUGUACUUUACUUUGUAUUAUGU | TTTT | 599 |
| Exon | | | | | |
| 52 | | | | | |
| Human- | 14 | 1 | UAAAGUACAAUUGUGAGACCAGCC | TTTT | 600 |
| Exon | | | | | |
| 52 | | | | | |
| Human- | 15 | 1 | GUAAAGUACAAUUGUGAGACCAGC | TTTG | 601 |
| Exon | | | | | |
| 52 | | | | | |
| Human- | 16 | 1 | GUAUUCCUUUUACAUAAUACAAAG | TTTA | 602 |
| Exon | | | | | |
| 52 | | | | | |
| Human- | 17 | 1 | GUUGUGUAUUCCUUUUACAUAAUA | TTTG | 603 |
| Exon | | | | | |
| 52 | | | | | |
| Human- | 18 | 1 | AUCCUGCAUUGUUGCCUGUAAGAA | TTTG | 604 |
| Exon | | | | | |
| 52 | | | | | |
| Human- | 19 | 1 | UUCCAACUGGGGACGCCUCUGUUC | TTTG | 605 |
| Exon | | | | | |
| 52 | | | | | |
| Human- | 20 | −1 | UUGGAAGAACUCAUUACCGCUGCC | TTTG | 606 |
| Exon | | | | | |
| 52 | | | | | |
| Human- | 21 | −1 | UCAUUACCGCUGCCCAAAAUUUGA | TTTT | 607 |
| Exon | | | | | |
| 52 | | | | | |
| Human- | 22 | 1 | CUCUUGAUUGCUGGUCUUGUUUUU | TTTG | 608 |
| Exon | | | | | |
| 52 | | | | | |
| Human- | 23 | −1 | GUUUUUUAACAAGCAUGGGACACA | TTTG | 609 |
| Exon | | | | | |
| 52 | | | | | |
| Human- | 24 | 1 | CUUUGUGUGUCCCAUGCUUGUUAA | TTTT | 610 |
| Exon | | | | | |
| 52 | | | | | |
| Human- | 25 | 1 | GCUUUGUGUGUCCCAUGCUUGUUA | TTTT | 611 |
| Exon | | | | | |
| 52 | | | | | |
| Human- | 26 | 1 | UGCUUUGUGUGUCCCAUGCUUGUU | TTTT | 612 |
| Exon | | | | | |
| 52 | | | | | |
| Human- | 27 | 1 | UUGCUUUGUGUGUCCCAUGCUUGU | TTTA | 613 |
| Exon | | | | | |
| 52 | | | | | |
| Human- | 28 | −1 | AGCAAGAUGCAUGACAAGUUUCAA | TTTA | 614 |
| Exon | | | | | |
| 52 | | | | | |
| Human- | 29 | −1 | GCAAGAUGCAUGACAAGUUUCAAU | TTTT | 615 |
| Exon | | | | | |
| 52 | | | | | |
| Human- | 30 | −1 | CAAGAUGCAUGACAAGUUUCAAUA | TTTT | 616 |
| Exon | | | | | |
| 52 | | | | | |
| Human- | 31 | 1 | GAUAUAUGAACUUAAGUUUUUAUU | TTTC | 617 |
| Exon | | | | | |
| 52 | | | | | |
| Human- | 1 | −1 | AUAGAAAUCCAAUAAUAUAUUCAC | TTTG | 618 |
| Exon | | | | | |
| 50 | | | | | |
| Human- | 2 | −1 | AUUAAGAUGUUCAUGAAUUAUCUU | TTTG | 619 |
| Exon | | | | | |
| 50 | | | | | |
| Human- | 3 | −1 | UAAGUAAUGUGUAUGCUUUUCUGU | TTTA | 620 |
| Exon | | | | | |
| 50 | | | | | |
| Human- | 4 | 1 | AUCUUCUAACUUCCUCUUUAACAG | TTTT | 621 |
| Exon | | | | | |
| 50 | | | | | |
| Human- | 5 | 1 | GAUCUUCUAACUUCCUCUUUAACA | TTTC | 622 |
| Exon | | | | | |
| 50 | | | | | |
| Human- | 6 | −1 | AUCUGAGCUCUGAGUGGAAGGCGG | TTTA | 623 |
| Exon | | | | | |
| 50 | | | | | |
| Human- | 7 | −1 | ACCGUUUACUUCAAGAGCUGAGGG | TTTG | 624 |
| Exon | | | | | |
| 50 | | | | | |
| Human- | 8 | 1 | CUGCUUUGCCCUCAGCUCUUGAAG | TTTA | 625 |
| Exon | | | | | |
| 50 | | | | | |
| Human- | 9 | −1 | UCUCUUUGGCUCUAGCUAUUUGUU | TTTG | 626 |
| Exon | | | | | |
| 50 | | | | | |
| Human- | 10 | −1 | CUCUUUGGCUCUAGCUAUUUGUUC | TTTT | 627 |
| Exon | | | | | |
| 50 | | | | | |
| Human- | 11 | 1 | CACUUUUGAACAAAUAGCUAGAGC | TTTG | 628 |
| Exon | | | | | |
| 50 | | | | | |
| Human- | 12 | 1 | UCACUUCAUAGUUGCACUUUUGAA | TTTG | 629 |
| Exon | | | | | |
| 50 | | | | | |
| Human- | 13 | −1 | AUGAAGUGAUGACUGGGUGAGAGA | TTTC | 630 |
| Exon | | | | | |
| 50 | | | | | |
| Human- | 14 | −1 | UGAAGUGAUGACUGGGUGAGAGAG | TTTT | 631 |
| Exon | | | | | |
| 50 | | | | | |
| Human- | 1 | 1 | AAGAGAAAAauauauauauauaua | TTTG | 632 |
| Exon | | | | | |
| 43 | | | | | |
| Human- | 2 | 1 | GAAUUAGCUGUCUAUAGAAAGAGA | tTTT | 633 |
| Exon | | | | | |
| 43 | | | | | |
| Human- | 3 | 1 | UGAAUUAGCUGUCUAUAGAAAGAG | TTTT | 634 |
| Exon | | | | | |
| 43 | | | | | |
| Human- | 4 | −1 | AGCUAAUUCAUUUUUUUACUGUUU | TTTA | 635 |
| Exon | | | | | |
| 43 | | | | | |
| Human- | 5 | 1 | AUGAAUUAGCUGUCUAUAGAAAGA | TTTC | 636 |
| Exon | | | | | |
| 43 | | | | | |
| Human- | 6 | −1 | GCUAAUUCAUUUUUUUACUGUUUU | TTTT | 637 |
| Exon | | | | | |
| 43 | | | | | |
| Human- | 7 | 1 | AAAAAAAUGAAUUAGCUGUCUAUA | TTTC | 638 |
| Exon | | | | | |
| 43 | | | | | |
| Human- | 8 | −1 | UUAAAAUUUUUAUAUUACAGAAUA | TTTA | 639 |
| Exon | | | | | |
| 43 | | | | | |
| Human- | 9 | −1 | UAAAAUUUUUAUAUUACAGAAUAU | TTTT | 640 |
| Exon | | | | | |
| 43 | | | | | |
| Human- | 10 | 1 | AUAUAAAAAUUUUAAAACAGUAAA | TTTT | 641 |
| Exon | | | | | |
| 43 | | | | | |
| Human- | 11 | 1 | AAUAUAAAAAUUUUAAAACAGUAA | TTTT | 642 |
| Exon | | | | | |
| 43 | | | | | |
| Human- | 12 | 1 | UAAUAUAAAAAUUUUAAAACAGUA | TTTT | 643 |
| Exon | | | | | |
| 43 | | | | | |
| Human- | 13 | 1 | GUAAUAUAAAAAUUUUAAAACAGU | TTTT | 644 |
| Exon | | | | | |
| 43 | | | | | |
| Human- | 14 | 1 | UGUAAUAUAAAAAUUUUAAAACAG | TTTA | 645 |
| Exon | | | | | |
| 43 | | | | | |
| Human- | 15 | 1 | UAUAUUCUGUAAUAUAAAAAUUUU | TTTT | 646 |
| Exon | | | | | |
| 43 | | | | | |
| Human- | 16 | 1 | UUAUAUUCUGUAAUAUAAAAAUUU | TTTA | 647 |
| Exon | | | | | |
| 43 | | | | | |
| Human- | 17 | −1 | CAGAAUAUAAAAGAUAGUCUACAA | TTTG | 648 |
| Exon | | | | | |
| 43 | | | | | |
| Human- | 18 | 1 | CUAUCUUUUAUAUUCUGUAAUAUA | TTTT | 649 |
| Exon | | | | | |
| 43 | | | | | |
| Human- | 19 | 1 | ACUAUCUUUUAUAUUCUGUAAUAU | TTTT | 650 |
| Exon | | | | | |
| 43 | | | | | |
| Human- | 20 | 1 | GACUAUCUUUUAUAUUCUGUAAUA | TTTA | 651 |
| Exon | | | | | |
| 43 | | | | | |
| Human- | 21 | −1 | CAUAGCAAGAAGACAGCAGCAUUG | TTTG | 652 |
| Exon | | | | | |
| 43 | | | | | |
| Human- | 22 | 1 | CAUUUUGUUAACUUUUUCCCAUUG | TTTC | 653 |
| Exon | | | | | |
| 43 | | | | | |
| Human- | 23 | −1 | CAUAUAUUUUUCUUGAUACUUGCA | TTTC | 654 |
| Exon | | | | | |
| 43 | | | | | |
| Human- | 24 | 1 | AAAUCAUUUCUGCAAGUAUCAAGA | TTTT | 655 |
| Exon | | | | | |
| 43 | | | | | |
| Human- | 25 | 1 | CAAAUCAUUUCUGCAAGUAUCAAG | TTTT | 656 |
| Exon | | | | | |
| 43 | | | | | |
| Human- | 26 | 1 | ACAAAUCAUUUCUGCAAGUAUCAA | TTTC | 657 |
| Exon | | | | | |
| 43 | | | | | |
| Human- | 27 | 1 | AUAAAUUCUACAGUUCCCUGAAAA | TTTG | 658 |
| Exon | | | | | |
| 43 | | | | | |
| Human- | 28 | −1 | GAAUUUAUUUCAGUACCCUCCAUG | TTTC | 659 |
| Exon | | | | | |
| 43 | | | | | |
| Human- | 29 | −1 | AAUUUAUUUCAGUACCCUCCAUGG | TTTT | 660 |
| Exon | | | | | |
| 43 | | | | | |
| Human- | 30 | 1 | UGAAAUAAAUUCUACAGUUCCCUG | TTTT | 661 |
| Exon | | | | | |
| 43 | | | | | |
| Human- | 31 | −1 | AUUUAUUUCAGUACCCUCCAUGGA | TTTT | 662 |
| Exon | | | | | |
| 43 | | | | | |
| Human- | 32 | 1 | CUGAAAUAAAUUCUACAGUUCCCU | TTTC | 663 |
| Exon | | | | | |
| 43 | | | | | |
| Human- | 33 | −1 | UUUAUUUCAGUACCCUCCAUGGAA | TTTT | 664 |
| Exon | | | | | |
| 43 | | | | | |
| Human- | 34 | −1 | UACCCUCCAUGGAAAAAAGACAGG | TTTC | 665 |
| Exon | | | | | |
| 43 | | | | | |
| Human- | 35 | −1 | ACCCUCCAUGGAAAAAAGACAGGG | TTTT | 666 |
| Exon | | | | | |
| 43 | | | | | |
| Human- | 36 | −1 | CCCUCCAUGGAAAAAAGACAGGGA | TTTT | 667 |
| Exon | | | | | |
| 43 | | | | | |
| Human- | 37 | 1 | UUUUUUCCAUGGAGGGUACUGAAA | TTTA | 668 |
| Exon | | | | | |
| 43 | | | | | |
| Human- | 38 | 1 | UGUCUUUUUUCCAUGGAGGGUACU | TTTC | 669 |
| Exon | | | | | |
| 43 | | | | | |
| Human- | 1 | 1 | CCUUGAGCAAGAACCAUGCAAACU | TTTA | 670 |
| Exon 6 | | | | | |
| Human- | 2 | −1 | UGCUCAAGGAAUGCAUUUUCUUAU | TTTC | 671 |
| Exon 6 | | | | | |
| Human- | 3 | −1 | GCUCAAGGAAUGCAUUUUCUUAUG | TTTT | 672 |
| Exon 6 | | | | | |
| Human- | 4 | 1 | UGCAUUCCUUGAGCAAGAACCAUG | TTTG | 673 |
| Exon 6 | | | | | |
| Human- | 5 | −1 | GAAAAUUUAUUUCCACAUGUAGGU | TTTG | 674 |
| Exon 6 | | | | | |
| Human- | 6 | −1 | AAAAUUUAUUUCCACAUGUAGGUC | TTTT | 675 |
| Exon 6 | | | | | |
| Human- | 7 | −1 | AAAUUUAUUUCCACAUGUAGGUCA | TTTT | 676 |
| Exon 6 | | | | | |
| Human- | 8 | 1 | CAUGUGGAAAUAAAUUUUCAUAAG | TTTT | 677 |
| Exon 6 | | | | | |
| Human- | 9 | 1 | ACAUGUGGAAAUAAAUUUUCAUAA | TTTC | 678 |
| Exon 6 | | | | | |
| Human- | 10 | −1 | CCACAUGUAGGUCAAAAAUGUAAU | TTTC | 679 |
| Exon 6 | | | | | |
| Human- | 11 | −1 | CACAUGUAGGUCAAAAAUGUAAUG | TTTT | 680 |
| Exon 6 | | | | | |
| Human- | 12 | −1 | ACAUGUAGGUCAAAAAUGUAAUGA | TTTT | 681 |
| Exon 6 | | | | | |
| Human- | 13 | 1 | ACAUUUUUGACCUACAUGUGGAAA | TTTA | 682 |
| Exon 6 | | | | | |
| Human- | 14 | 1 | CAUUACAUUUUUGACCUACAUGUG | TTTC | 683 |
| Exon 6 | | | | | |
| Human- | 15 | −1 | AAAAAUAUCAUGGCUGGAUUGCAA | TTTG | 684 |
| Exon 6 | | | | | |
| Human- | 16 | −1 | GCUGGAUUGCAACAAACCAACAGU | TTTC | 685 |
| Exon 6 | | | | | |
| Human- | 17 | −1 | CUGGAUUGCAACAAACCAACAGUG | TTTT | 686 |
| Exon 6 | | | | | |
| Human- | 18 | 1 | CCUAUGACUAUGGAUGAGAGCAUU | TTTG | 687 |
| Exon 6 | | | | | |
| Human- | 19 | −1 | UAGGUAAGAAGAUUACUGAGACAU | TTTA | 688 |
| Exon 6 | | | | | |
| Human- | 20 | −1 | AUUACUGAGACAUUAAAUAACUUG | TTTA | 689 |
| Exon 6 | | | | | |
| Human- | 21 | −1 | UUACUGAGACAUUAAAUAACUUGU | TTTT | 690 |
| Exon 6 | | | | | |
| Human- | 22 | 1 | GGGGAAAAAUAUGUCAUCAGAGUC | TTTA | 691 |
| Exon 6 | | | | | |
| Human- | 23 | 1 | CAUGAUCUGGAACCAUACUGGGGA | TTTT | 692 |
| Exon 6 | | | | | |
| Human- | 24 | 1 | ACAUGAUCUGGAACCAUACUGGGG | TTTT | 693 |
| Exon 6 | | | | | |
| Human- | 25 | 1 | GACAUGAUCUGGAACCAUACUGGG | TTTC | 694 |
| Exon 6 | | | | | |
| Human- | 1 | 1 | uacacacauacacaAAGACAAAUA | TTTA | 695 |
| Exon 7 | | | | | |
| Human- | 2 | 1 | uacacauacacacauacacaAAGA | TTTG | 696 |
| Exon 7 | | | | | |
| Human- | 3 | 1 | aacacauacacauacacacauaca | TTtg | 697 |
| Exon 7 | | | | | |
| Human- | 4 | 1 | AUUCCAGUCAAAUAGGUCUGGCCU | ttTT | 698 |
| Exon 7 | | | | | |
| Human- | 5 | 1 | UAUUCCAGUCAAAUAGGUCUGGCC | tTTA | 699 |
| Exon 7 | | | | | |
| Human- | 6 | 1 | GCUGGCAAACCACACUAUUCCAGU | TTTG | 700 |
| Exon 7 | | | | | |
| Human- | 7 | 1 | AGUCGUUGUGUGGCUGACUGCUGG | TTTG | 701 |
| Exon 7 | | | | | |
| Human- | 8 | −1 | CGCCAGAUAUCAAUUAGGCAUAGA | TTTC | 702 |
| Exon 7 | | | | | |
| Human- | 9 | −1 | AAACUACUCGAUCCUGAAGGUUGG | TTTA | 703 |
| Exon 7 | | | | | |
| Human- | 10 | 1 | CAUACUAAAAGCAGUGGUAGUCCA | TTTC | 704 |
| Exon 7 | | | | | |
| Human- | 11 | 1 | GAAAACAUUAAACUCUACCAUACU | TTTT | 705 |
| Exon 7 | | | | | |
| Human- | 12 | 1 | UGAAAACAUUAAACUCUACCAUAC | TTTA | 706 |
| Exon 7 | | | | | |
| Human- | 1 | −1 | UUGUUCAUUAUCCUUUUAGAGUCU | TTTG | 707 |
| Exon 8 | | | | | |
| Human- | 2 | 1 | AAAGGAUAAUGAACAAAUCAAAGU | TTTA | 708 |
| Exon 8 | | | | | |
| Human- | 3 | −1 | UAUCCUUUUAGAGUCUCAAAUAUA | TTTC | 709 |
| Exon 8 | | | | | |
| Human- | 4 | 1 | ACUCUAAAAGGAUAAUGAACAAAU | TTTG | 710 |
| Exon 8 | | | | | |
| Human- | 5 | −1 | UUUUAGAGUCUCAAAUAUAGAAAC | TTTG | 711 |
| Exon 8 | | | | | |
| Human- | 6 | −1 | UUUAGAGUCUCAAAUAUAGAAACC | TTTT | 712 |
| Exon 8 | | | | | |
| Human- | 7 | −1 | UUAGAGUCUCAAAUAUAGAAACCA | TTTT | 713 |
| Exon 8 | | | | | |
| Human- | 8 | 1 | UUGAGACUCUAAAAGGAUAAUGAA | TTTG | 714 |
| Exon 8 | | | | | |
| Human- | 9 | 1 | UUUGGUUUCUAUAUUUGAGACUCU | TTTT | 715 |
| Exon 8 | | | | | |
| Human- | 10 | 1 | UUUUGGUUUCUAUAUUUGAGACUC | TTTA | 716 |
| Exon 8 | | | | | |
| Human- | 11 | −1 | AGCAUUGAAGCCAUCCAGGAAGUG | TTTC | 717 |
| Exon 8 | | | | | |
| Human- | 12 | 1 | GCUUCAAUGCUCACUUGUUGAGGC | TTTT | 718 |
| Exon 8 | | | | | |
| Human- | 13 | 1 | GGCUUCAAUGCUCACUUGUUGAGG | TTTG | 719 |
| Exon 8 | | | | | |
| Human- | 14 | −1 | AGUGGAAAUGUUGCCAAGGCCACC | TTTA | 720 |
| Exon 8 | | | | | |
| Human- | 15 | −1 | GUUGCCAAGGCCACCUAAAGUGAC | TTTA | 721 |
| Exon 8 | | | | | |
| Human- | 16 | −1 | GAAGAACAUUUUCAGUUACAUCAU | TTTG | 722 |
| Exon 8 | | | | | |
| Human- | 17 | −1 | AUCAAAUGCACUAUUCUCAACAGG | TTTA | 723 |
| Exon 8 | | | | | |
| Human- | 18 | 1 | AUAGUGCAUUUGAUGAUGUAACUG | TTTT | 724 |
| Exon 8 | | | | | |
| Human- | 19 | 1 | AAUAGUGCAUUUGAUGAUGUAACU | TTTC | 725 |
| Exon 8 | | | | | |
| Human- | 20 | −1 | ACUAUUCUCAACAGGUAAAGUGUG | TTTA | 726 |
| Exon 8 | | | | | |
| Human- | 21 | 1 | UACCUAAAAAUGCAUAUAAAACAG | TTTT | 727 |
| Exon 8 | | | | | |
| Human- | 22 | 1 | AUACCUAAAAAUGCAUAUAAAACA | TTTC | 728 |
| Exon 8 | | | | | |
| Human- | 23 | 1 | CACGUAAUACCUAAAAAUGCAUAU | TTTT | 729 |
| Exon 8 | | | | | |
| Human- | 24 | 1 | GCACGUAAUACCUAAAAAUGCAUA | TTTA | 730 |
| Exon 8 | | | | | |
| Human- | 25 | 1 | auauauauGUGCACGUAAUACCUA | TTTT | 731 |
| Exon 8 | | | | | |
| Human- | 26 | 1 | uauauauauGUGCACGUAAUACCU | TTTT | 732 |
| Exon 8 | | | | | |
| Human- | 27 | 1 | auauauauauGUGCACGUAAUACC | TTTA | 733 |
| Exon 8 | | | | | |
| Human- | 1 | −1 | CUGCACAAUAUUAUAGUUGUUGCU | TTTA | 734 |
| Exon | | | | | |
| 55 | | | | | |
| Human- | 2 | 1 | AUAAAAAGAGAAAGAUGGAGGAAC | TTTA | 735 |
| Exon | | | | | |
| 55 | | | | | |
| Human- | 3 | 1 | CACCUAGUGAACUCCAUAAAAAGA | TTTC | 736 |
| Exon | | | | | |
| 55 | | | | | |
| Human- | 4 | 1 | AUGGUGCACCUAGUGAACUCCAUA | TTTT | 737 |
| Exon | | | | | |
| 55 | | | | | |
| Human- | 5 | 1 | AAUGGUGCACCUAGUGAACUCCAU | TTTT | 738 |
| Exon | | | | | |
| 55 | | | | | |
| Human- | 6 | 1 | GAAUGGUGCACCUAGUGAACUCCA | TTTA | 739 |
| Exon | | | | | |
| 55 | | | | | |
| Human- | 7 | 1 | GACCAAAUGUUCAGAUGCAAUUAU | TTTA | 740 |
| Exon | | | | | |
| 55 | | | | | |
| Human- | 8 | 1 | UCGCUCACUCACCCUGCAAAGGAC | TTTG | 741 |
| Exon | | | | | |
| 55 | | | | | |
| Human- | 9 | −1 | AGUGAGCGAGAGGCUGCUUUGGAA | TTTC | 742 |
| Exon | | | | | |
| 55 | | | | | |
| Human- | 10 | 1 | GCAGCCUCUCGCUCACUCACCCUG | TTTG | 743 |
| Exon | | | | | |
| 55 | | | | | |
| Human- | 11 | 1 | UUGCAGUAAUCUAUGAGUUUCUUC | TTTG | 744 |
| Exon | | | | | |
| 55 | | | | | |
| Human- | 12 | −1 | CUGCAACAGUUCCCCCUGGACCUG | TTTC | 745 |
| Exon | | | | | |
| 55 | | | | | |
| Human- | 13 | −1 | UGCAACAGUUCCCCCUGGACCUGG | TTTT | 746 |
| Exon | | | | | |
| 55 | | | | | |
| Human- | 14 | −1 | UUUCUUGCCUGGCUUACAGAAGCU | TTTC | 747 |
| Exon | | | | | |
| 55 | | | | | |
| Human- | 15 | 1 | UUUCAGCUUCUGUAAGCCAGGCAA | TTTC | 748 |
| Exon | | | | | |
| 55 | | | | | |
| Human- | 16 | −1 | GUCCUACAGGAUGCUACCCGUAAG | TTTC | 749 |
| Exon | | | | | |
| 55 | | | | | |
| Human- | 17 | −1 | GGCUCCUAGAAGACUCCAAGGGAG | TTTA | 750 |
| Exon | | | | | |
| 55 | | | | | |
| Human- | 18 | −1 | GCUCCUAGAAGACUCCAAGGGAGU | TTTT | 751 |
| Exon | | | | | |
| 55 | | | | | |
| Human- | 19 | −1 | CUCCAAGGGAGUAAAAGAGCUGAU | TTTC | 752 |
| Exon | | | | | |
| 55 | | | | | |
| Human- | 20 | 1 | UGGAUCCACAAGAGUGCUAAAGCG | TTTC | 753 |
| Exon | | | | | |
| 55 | | | | | |
| Human- | 21 | 1 | GUUCAAUUGGAUCCACAAGAGUGC | TTTA | 754 |
| Exon | | | | | |
| 55 | | | | | |
| Human- | 22 | −1 | UACUUGUAACUGACAAGCCAGGGA | TTTG | 755 |
| Exon | | | | | |
| 55 | | | | | |
| Human- | 23 | −1 | ACUUGUAACUGACAAGCCAGGGAC | TTTT | 756 |
| Exon | | | | | |
| 55 | | | | | |
| Human- | 24 | −1 | GUAACUGACAAGCCAGGGACAAAA | TTTG | 757 |
| Exon | | | | | |
| 55 | | | | | |
| Human- | 25 | −1 | UAACUGACAAGCCAGGGACAAAAC | TTTT | 758 |
| Exon | | | | | |
| 55 | | | | | |
| Human- | 26 | 1 | UCCCUGGCUUGUCAGUUACAAGUA | TTTG | 759 |
| Exon | | | | | |
| 55 | | | | | |
| Human- | | 1 | CAGAGUAACAGUCUGAGUAGGAGc | TTTA | 760 |
| G1- | | | | | |
| exon51 | | | | | |
| Human- | | 1 | uacuuuguuuagcaauacauggua | TTTC | 761 |
| G2- | | | | | |
| exon51 | | | | | |
| Human- | | −1 | uggcucaaauuguuacucuucaau | TTTA | 762 |
| G3- | | | | | |
| exon51 | | | | | |
| mouse- | | 1 | CUUUCAAagancuuugcagagccu | TTTG | 763 |
| Exon | | | | | |
| 23-G1 | | | | | |
| mouse- | | 1 | guugaaGCCAUUUUGUUGCUCUUU | TTTG | 764 |
| Exon | | | | | |
| 23-G2 | | | | | |
| mouse- | | 1 | guugaaGCCAUUUUAUUGCUCUUU | TTTG | 765 |
| Exon | | | | | |
| 23-G3 | | | | | |
| mouse- | | −1 | uuuugagGCUCUGCAAAGUUCUUU | TTTC | 766 |
| Exon | | | | | |
| 23-G4 | | | | | |
| mouse- | | −1 | aguuauuaaugcauagauauucag | TTTA | 767 |
| Exon | | | | | |
| 23-G5 | | | | | |
| mouse- | | −1 | uauaauaugcccuguaauauaaua | TTTC | 768 |
| Exon | | | | | |
| 23-G6 | | | | | |
| mouse- | | 1 | uaaaggccaaaccucggcuuaccU | TTTC | 769 |
| Exon | | | | | |
| 23-G7 | | | | | |
| mouse- | | 1 | ucaauaucuuugaaggacucuggg | TTTA | 770 |
| Exon | | | | | |
| 23-G8 | | | | | |

*In this table, upper case letters represent sgRNA nucleotides that align to the exon sequence of the gene. Lower case letters represent sgRNA nucleotides that align to the intron sequence of the gene.

### VI. SEQUENCE TABLES

TABLE 3
Sequence of primers for sgRNA targeting Dmd Exon
50 and Exon 79 to generate the mice models

| | | | SEQ |
|---|---|---|---|
| | Mouse | | ID |
| ID | Model | Sequence (5′-3′) | NO. |
| exon | Δex50 | CACCGAAATGATGAGTGAAGTTAT | 1 |
| 50_F1 | | AT | |
| exon | Δex50 | AAACATATAACTTCACTCATCATTT | 2 |
| 50_R1 | | C | |
| exon | Δex50 | CACCGGTTTGTTCAAAAGCGTGGCT | 3 |
| 50_F2 | | | |
| exon | Δex50 | AAACAGCCACGCTTTTGAACAAAC | 4 |
| 50_R2 | | | |
| exon79_F1 | Dmd-KI- | CACCGGACACAATGTAGGAAGCCT | 5 |
| | Luciferase | | |
| exon79_R1 | Dmd-KI- | AAACAGGCTTCCTACATTGTGTCC | 6 |
| | Luciferase | | |

TABLE 4
Sequence of primers for in vitro
transcription of sgRNA

| | | | SEQ |
|---|---|---|---|
| | Mouse | | ID |
| ID | Model | Sequence (5′-3′) | NO. |
| exon | Δex50 | GAATTGTAATACGACTCACTATAGG | 7 |
| 50_T7-F1 | | AATGATGAGTGAAGTTATAT | |
| exon | Δex50 | GAATTGTAATACGACTCACTATAGG | 8 |
| 50_T7-F2 | | GTTTGTTCAAAAGCGTGGCT | |
| exon | Δex50 | AAAAGCACCGACTCGGTGCCAC | 9 |
| 50_T7-Rv | | | |
| exon | Δex50 | AAACAGCCACGCTTTTGAACAAAC | 10 |
| 50_R2 | | | |
| exon | Dmd-KI- | GAATTGTAATACGACTCACTGGAC | 11 |
| 79_T7-F1 | Luciferase | ACAATGTAGGAAGCCT | |
| exon | Dmd-KI- | AAAAGCACCGACTCGGTGCCAC | 12 |
| 79_T7-Rv | Luciferase | | |

TABLE 5
Sequence of primers for genotyping

| | | | SEQ |
|---|---|---|---|
| | Mouse | | ID |
| ID | Model | Sequence (5′-3′) | NO. |
| Geno50-F | Δx50 | GGATTGACTGAAATGATGGCCAAG | 13 |
| | | G | |
| Geno50-R | Δex50 | CTGCCACGATTACTCTGCTTCCAG | 14 |
| GenoKI/ | Dmd-KI- | AGCAGGCAGAGAAGGTGGTA | 15 |
| WT-F | Luciferase | | |
| GenoKI-R | Dmd-KI- | GGGCGTATCTCTTCATAGCCTT | 16 |
| | Luciferase | | |
| GenoWT-R | Dmd-KI- | GCGTGTGTGTTTGTTTAGG | 17 |
| | Luciferase | | |

TABLE 6
Sequence of primers for sgRNA targeting Dmd
Exon 51 for correction of reading frame

| | | | SEQ |
|---|---|---|---|
| | Mouse | | ID |
| ID | Model | Sequence (5′-3′) | NO. |
| exon | ex51-SA-Top | CACCGCACTAGAGTAACAGTCTGA | 771 |
| 51_F1 | | C | |
| exon | ex51-SA-Bottom | AAACCCAGTCAGACTGTTACTCTC | 772 |
| 51_F1 | | | |

TABLE 7
Sequence of primers for Amplicon Deep
Sequencing Analysis

| | | | SEQ |
|---|---|---|---|
| | Mouse | | ID |
| ID | Model | Sequence (5′-3′) | NO. |
| Amplicon | M-ex51- | TCGTCGGCAGCGTCAGATGTGTATA | 773 |
| Deep | Mi-seq-F | AGAGACAGGAAATTTTACCTCAAA | |
| Sequencing | | CTGTTGCTTC | |
| Amplicon | M-ex51- | GTCTCGTGGGCTCGGAGATGTGTAT | 774 |
| Deep | Mi-seq-R | AAGAGACAGGAGGGAAATGGAAA | |
| Sequencing | | GTGACAATATAC | |
| Amplicon | Univ- | AATGATACGGCGACCACCGAGATC | 775 |
| Deep | Miseq-BC- | TACACTCGTCGGCAGCGTC | |
| Sequencing | Fw-LA | | |
| Amplicon | BC1-LA | CAAGCAGAAGACGGCATACGAGAT | 776 |
| Deep | | ACATCGGTCTCGTGGGCTCGG | |
| Sequencing | | | |
| Amplicon | BC2-LA | CAAGCAGAAGACGGCATACGAGAT | 777 |
| Deep | | TGGTCAGTCTCGTGGGCTCGG | |
| Sequencing | | | |
| Amplicon | BC3-LA | CAAGCAGAAGACGGCATACGAGAT | 778 |
| Deep | | CACTGTGTCTCGTGGGCTCGG | |
| Sequencing | | | |
| Amplicon | BC4-LA | CAAGCAGAAGACGGCATACGAGAT | 779 |
| Deep | | ATTGGCGTCTCGTGGGCTCGG | |
| Sequencing | | | |
| Amplicon | BC5-LA | CAAGCAGAAGACGGCATACGAGAT | 780 |
| Deep | | GATCTGGTCTCGTGGGCTCGG | |
| Sequencing | | | |
| Amplicon | BC6-LA | CAAGCAGAAGACGGCATACGAGAT | 781 |
| Deep | | TACAAGGTCTCGTGGGCTCGG | |
| Sequencing | | | |
| Amplicon | BC7-LA | CAAGCAGAAGACGGCATACGAGAT | 782 |
| Deep | | CGTGATGTCTCGTGGGCTCGG | |
| Sequencing | | | |
| Amplicon | BC8-LA | CAAGCAGAAGACGGCATACGAGAT | 783 |
| Deep | | GCCTAAGTCTCGTGGGCTCGG | |
| Sequencing | | | |
| Amplicon | BC9-LA | CAAGCAGAAGACGGCATACGAGAT | 784 |
| Deep | | TCAAGTGTCTCGTGGGCTCGG | |
| Sequencing | | | |
| Amplicon | BC10-LA | CAAGCAGAAGACGGCATACGAGAT | 785 |
| Deep | | AGCTAGGTCTCGTGGGCTCGG | |
| Sequencing | | | |

### VII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Study Approval. All experimental procedures involving animals in this study were reviewed and approved by the University of Texas Southwestern Medical Center's Institutional Animal Care and Use Committee.

CRISPR/Cas9-mediated exon 50 deletion in mice. Two single-guide RNA (sgRNA) specific intronic regions surrounding exon 50 sequence of the mouse Dmd locus were cloned into vector px330 using the primers from Table 3. For the in vitro transcription of sgRNA, T7 promoter sequence was added to the sgRNA template by PCR using the primers from Table 4. The gel purified PCR products were used as template for in vitro transcription using the MEGAshortscript T7 Kit (Life Technologies). sgRNA were purified by MEGAclear kit (Life Technologies) and eluted with nuclease-free water (Ambion). The concentration of guide RNA was measured by a NanoDrop instrument (Thermo Scientific).

CRISPR/Cas9-mediated Homologous Recombination in Mice. A single-guide RNA (sgRNA) specific to the exon 79 sequence of the mouse Dmd locus was cloned into vector px330 using the primers from Table 3. For the in vitro transcription of sgRNA, T7 promoter sequence was added to the sgRNA template by PCR using the primers from Table 4. A donor vector containing the protease 2A and luciferase reporter sequence was constructed by incorporating short 5′ and 3′ homology arms specific to the Dmd gene locus.

Genotyping of ΔEx50 Mice and Dmd-Luciferase Mice. ΔEx50, Dmd-Luciferase and ΔEx50-Dmd-Luciferase mice were genotyped using primers encompassing the targeted region from Table 5. Tail biopsies were digested in 100 μL of 25-mM NaOH, 0.2-mM EDTA (pH 12) for 20 min at 95° C. Tails were briefly centrifuged followed by addition of 100 μL of 40-mM Tris.HCl (pH 5) and mixed to homogenize. Two microliters of this reaction was used for subsequent PCR reactions with the primers below, followed by gel electrophoresis.

Plasmids. The pSpCas9(BB)-2A-GFP (PX458) plasmid containing the human codon optimized SpCas9 gene with 2A-EGFP and the backbone of sgRNA was purchased from Addgene (Plasmid #48138). Cloning of sgRNA was done using Bbs I site.

AAV9 strategy and delivery to ΔEx50-KI-Luciferase mice. Dmd exon 51 sgRNAs were selected using crispr.mit.edu. sgRNA sequences were cloned into px330 using primers in Table 4. sgRNAs were tested in tissue culture using 10T1/2 cells as previously described (Long et al., 2016) before cloning into the rAAV9 backbone.

Prior to AAV9 injections, ΔEx50-KI-Luciferase mice were anesthetized by intraperitoneal (IP) injection of ketamine and xylazine anesthetic cocktail. For intramuscular (IM) injection, tibialis anterior (TA) muscle of P12 male ΔEx50 mice was injected with 50 μl of AAV9 (1E12 vg/ml) preparations, or saline solution.

Targeted deep DNA sequencing. PCR of genomic DNA from 10T1/2 mouse fibroblast was performed using primers designed against the respective target region and off-target sites (Table 5). A second round of PCR was used to add Illumina flowcell binding sequences and experiment-specific barcodes on the 5′ end of the primer sequence (Table 2). Before sequencing, DNA libraries were analyzed using a Bioanalyzer High Sensitivity DNA Analysis Kit (Agilent). Library concentration was then determined by qPCR using a KAPA Library Quantification Kit for Illumina platforms. The resulting PCR products were pooled and sequenced with 300 bp paired-end reads on an Illumina MiSeq instrument. Samples were demultiplexed according to assigned barcode sequences. FASTQ format data was analyzed using the CRISPResso software package version 1.0.8 (Pinello et al., 2016).

Western blot analysis. Western blot was performed as described previously (Long et al., 2016). Antibodies to dystrophin (1:1000, D8168, Sigma-Aldrich), luciferin (1:1000, Abcam ab21176), vinculin (1:1000, V9131, Sigma-Aldrich), goat anti-mouse and goat-anti rabbit HRP-conjugated secondary antibodies (1:3000, Bio-Rad) were used for the described experiments.

### Example 2—Results

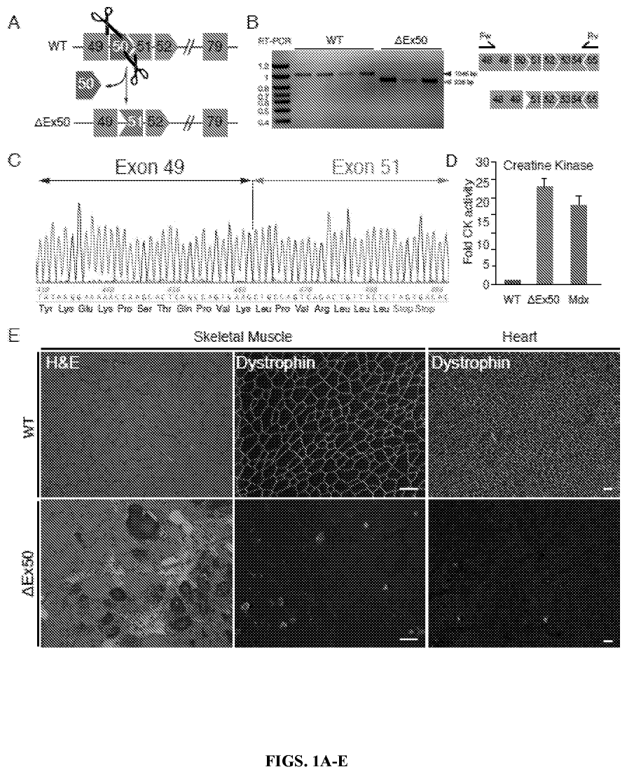

New Humanized model recapitulates muscle dystrophy phenotype. The first hot spot mutation region in DMD patients is the region between exon 45 to 51 where skipping of exon 51 would apply to the largest group (i.e., 13-14% of DMD patients). To investigate CRISPR/Cas9-mediated exon 51 skipping in vivo, a mimic of the human “hot spot” region was generated in a mouse model by deleting the exon 50 using CRISPR/Cas9 system directed by 2 single guide RNA (sgRNA) (FIG. 1A). The deletion of exon 50 was confirmed by DNA sequencing (FIG. 1B). The deletion of exon 50 placed the dystrophin gene out of frame leading to the absence of dystrophin protein in skeletal muscle and heart (FIG. 1C). Mice lacking exon 50 showed pronounced dystrophic muscle changes in 2 months-old mice. Serum analysis of delta-exon 50 mice shows a significant increase of creatine kinase (CK) level, which is a sign of muscle damage. Taken together, dystrophin protein expression, muscle histology and serum validated dystrophic phenotype of ΔEx50 mouse model.

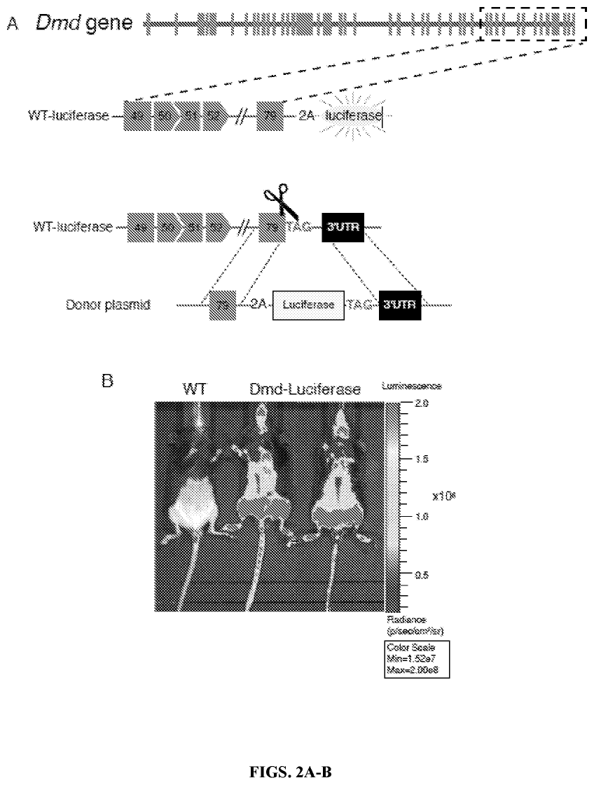

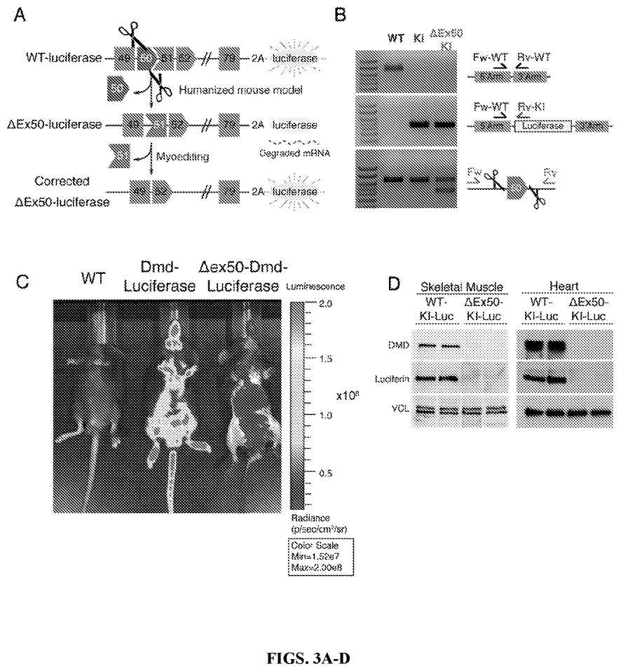

Humanized DMD reporter line. In an effort to facilitate the analysis of exon skipping strategies in vivo in a non-invasive way, reporter mice were generated by insertion of a Luciferase expression cassette into the 3′ end of the Dmd gene so that Luciferase would be translated in-frame with exon 79 of dystrophin, referred as Dmd-KI-Luciferase as shown in FIGS. 2A-B. To avoid the possibility that Luciferase might destabilize the dystrophin protein, a protease 2A was engineered at cleavage site between the proteins, which is auto-catalytically cleaved (FIG. 2A). Thus, the reporter protein will be released from dystrophin after translation. The reporter Dmd-luciferase reporter line were successfully generated and validated by DNA sequencing. The bioluminescence imaging of mice shows a high-expression level and muscle-specificity of Luciferase expression in the Dmd-Luciferase mice (FIG. 2B). To generate a ΔEx50-Dmd-luciferase reporter line mouse, 2 sgRNA were used to delete exon 50 in Dmd-luciferase reporter line (FIG. 3A). The deletion of exon 50 was confirmed by DNA sequencing. The deletion of exon 50 placed the dystrophin gene out of frame leading to the absence of dystrophin protein and decreased bioluminescence signal (FIG. 3C). Deletion of exon 50 placed the Dmd gene out of frame, preventing production of dystrophin protein in skeletal muscle and heart (FIG. 3D). Thus, since the Luciferase reporter protein expression is linked to the dystrophin translation the deletion of exon 50 leads to the absence of luciferin protein expression in ΔEx50-KI-Luciferase mice (FIG. 3D).

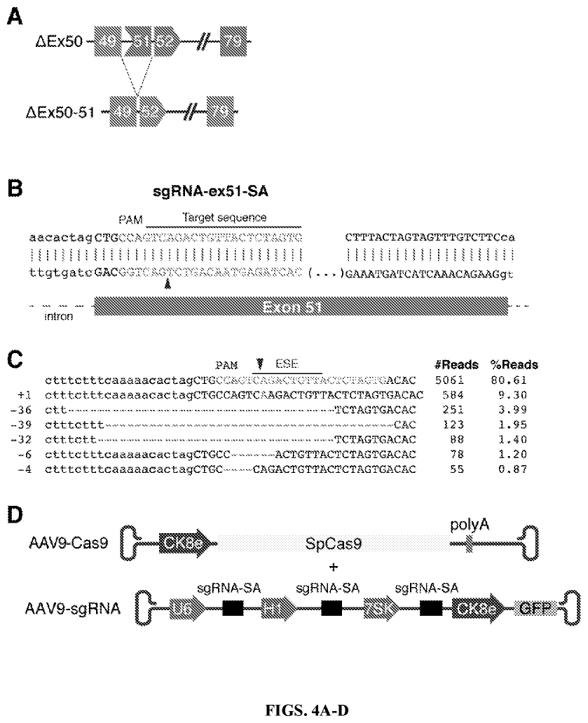

In vivo monitoring of correction of the dystrophin reading frame in ΔEx50-KI-Luciferase mice by a single DNA cut. To correct the dystrophin reading frame in ΔEx50-KI-Luciferase mice (FIG. 4A), sgRNA were designed to target a region adjacent to the exon 51 splice acceptor site (referred to as sgRNA-SA) (FIG. 4B). S. pyogenes Cas9 that requires NAG/NGG as a proto-spacer adjacent motif (PAM) sequence to generate a double-strand DNA break was used for the in vivo correction.

First, the DNA cutting activity of Cas9 coupled with sgRNA-SA was evaluated in 10T1/2 mouse fibroblasts. To investigate the type of mutations generated by Cas9 coupled with sgRNA-SA, genomic deep-sequencing analysis was performed. The sequencing analysis revealed that 9.3% of mutations contained a single adenosine (A) insertion 4 nucleotides 3′ of the PAM sequence and 7.3% contained deletions covering the splice acceptor site and a highly-predicted ESE site for exon 51 (FIG. 4C).

For the in vivo delivery of Cas9 and sgRNA-SA to skeletal muscle and heart tissue, adeno-associated virus 9 (AAV9) was used, which displays preferential tropism for these tissues. To further enhance muscle-specific expression, an AAV9-Cas9 vector (CK8e-Cas9-shortPolyA), which contains a muscle-specific creatine kinase (CK) regulatory cassette was used, referred to as the CK8e promoter, which is highly specific for expression in muscle and heart (FIG. 4D). This 436 bp muscle-specific cassette and the 4101 bp Cas9 cDNA, together, are within the packaging limit of AAV9. Expression of each sgRNA was driven by three RNA polymerase III promoters (U6, H1 and 7SK) (FIG. 4D).

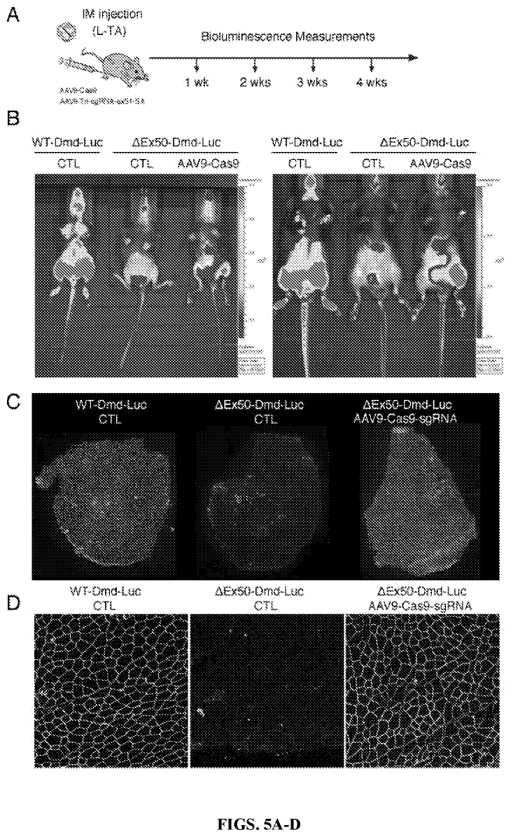

Following intra-muscular (IM) injection of mice at postnatal day (P) 12 with 5E10 AAV9 viral genomes (vg) in left tibialis anterior (TA) muscles were analyzed and monitored by bioluminescence for 4 weeks (FIG. 5A). The in vivo bioluminescence analysis showed appearance of signal in the injected leg 1 week after injection. The signal progressively increased over the following weeks expanding to the entire hindlimb muscles (FIG. 5B).

Histological analysis of AAV9-injected TA muscle was performed to evaluate the number of fibers that expressed dystrophin and the correlation with the bioluminescence signal. Dystrophin immunohistochemistry of muscle from ΔEx50-KI-Luciferase mice injected with AAV9-SA revealed restoration of dystrophin (FIGS. 5C-D). Taken together, these results demonstrate an in vivo assessment of dystrophin reading frame correction in ΔEx50-KI-Luciferase mice. ΔEx50-KI-Luciferase mice will be useful as a platform for testing many different strategies for amelioration of DMD pathogenesis.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
Angel et al., Mol. Cell. Biol., 7:2256, 1987a.
Angel et al., Cell, 49:729, 1987b.
Aartsma-Rus et al., Hum. Mutat. 30, 293-299, 2009.
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-7, 1997.
Baskin et al., EMBO Mol Med 6, 1610-1621, 2014.
Benvenisty and Neshif, Proc. Natl. Acad. Sci. USA, 83:9551-9555, 1986.
Berkhout et al., Cell, 59:273-282, 1989.
Bhaysar et al., Genomics, 35(1):11-23, 1996.
Bikard et al., Nucleic Acids Res. 41(15): 7429-7437, 2013.
Blanar et al., EMBO J., 8:1139, 1989.
Bodine and Ley, EMBO J., 6:2997, 1987.
Boshart et al., Cell, 41:521, 1985.
Bostick et al., Mol Ther 19, 1826-1832, 2011.
Bosze et al., EMBO J., 5(7):1615-1623, 1986.
Braddock et al., Cell, 58:269, 1989.
Brinster et al., Proc. Natl. Acad. Sci. USA, 82(13):4438-4442, 1985.
Bulla and Siddiqui, J. Virol., 62:1437, 1986.
Burridge et al., Nat. Methods 11, 855-860, 2014.
Bushby et al., Lancet Neurol., 9(1): 77-93 (2010).
Bushby et al., Lancet Neurol., 9(2): 177-198 (2010).
Campbell & Kahl, Nature 338, 259-262, 1989.
Campbell and Villarreal, Mol. Cell. Biol., 8:1993, 1988.
Campere and Tilghman, Genes and Dev., 3:537, 1989.
Campo et al., Nature, 303:77, 1983.
Celander and Haseltine, J. Virology, 61:269, 1987.
Celander et al., J. Virology, 62:1314, 1988.
Chandler et al., Cell, 33:489, 1983.
Chang et al., Mol. Cell. Biol., 9:2153, 1989.
Chang et al., Stem Cells., 27:1042-1049, 2009.
Chatterjee et al., Proc. Natl. Acad. Sci. USA, 86:9114, 1989.
Chen and Okayama, Mol. Cell Biol., 7:2745-2752, 1987.
Cho et al., Nat. Biotechnol. 31(3): 230-232, 2013.
Choi et al., Cell, 53:519, 1988.
Cirak et al., The Lancet 378, 595-605, 2011.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
DeWitt et al., Sci Transl Med 8, 360ra134-360ra134, 2016.
Donnelly et al., J. Gen. Virol. 82, 1027-1041, 2001.
Dubensky et al., Proc. Natl. Acad. Sci. USA, 81:7529-7533, 1984.
Edbrooke et al., Mol. Cell. Biol., 9:1908, 1989.
Edlund et al., Science, 230:912-916, 1985.
EP 0273085
Fechheimer et al., Proc Natl. Acad. Sci. USA, 84:8463-8467, 1987.
Feng and Holland, Nature, 334:6178, 1988.
Ferkol et al., FASEB J., 7:1081-1091, 1993.
Firak et al., Mol. Cell. Biol., 6:3667, 1986.
Foecking et al., Gene, 45(1):101-105, 1986.
Fonfara et al., Nature 532, 517-521, 2016.
Fraley et al., Proc Natl. Acad. Sci. USA, 76:3348-3352, 1979
Franz et al., Cardoscience, 5(4):235-43, 1994.
Friedmann, Science, 244:1275-1281, 1989.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goncalves et al., Mol Ther, 19(7): 1331-1341 (2011).
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Gopal-Srivastava et al., J. Mol. Cell. Biol., 15(12):7081-90, 1995.
Graham and Prevec, In: Methods in Molecular Biology: Gene Transfer and Expression Protocol, Murray (Ed.), Humana Press, Clifton, N.J., 7:109-128, 1991.
Graham and van der Eb, Virology, 52:456-467, 1973.
Graham et al., J. Gen. Virol., 36:59-72, 1977.
Greene et al., Immunology Today, 10:272, 1989
Grosschedl and Baltimore, Cell, 41:885, 1985.
Grunhaus and Horwitz, Seminar in Virology, 3:237-252, 1992.
Harland and Weintraub, J. Cell Biol., 101:1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Nat'l Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Nat'l. Acad. Sci. USA 90:2812-2816, 1993.
Hirochika et al., J. Virol., 61:2599, 1987.
Holbrook et al., Virology, 157:211, 1987.
Hollinger & Chamberlain, Current Opinion in Neurology 28, 522-527, 2015.
Horlick and Benfield, Mol. Cell. Biol., 9:2396, 1989.
Horwich et al., J. Virol., 64:642-650, 1990.
Hsu et al., Natl Biotechnol. 31:827-832, 2013
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jinek et al., Science 337, 816-821, 2012.
Johnson et al., Mol. Cell. Biol., 9:3393, 1989.
Jones and Shenk, Cell, 13:181-188, 1978.
Kadesch and Berg, Mol. Cell. Biol., 6:2593, 1986.
Kaneda et al., Science, 243:375-378, 1989.
Karin et al., Mol. Cell. Biol., 7:606, 1987.
Karlsson et al., EMBO J., 5:2377-2385, 1986.
Katinka et al., Cell, 20:393, 1980.
Kato et al., J Biol Chem., 266(6):3361-3364, 1991.
Kawamoto et al., Mol. Cell. Biol., 8:267, 1988.
Kelly et al., J. Cell Biol., 129(2):383-96, 1995.
Kiledjian et al., Mol. Cell. Biol., 8:145, 1988.
Kim et al., Nature Biotechnology, 1-2, 2016.
Kim et al., Nature Biotechnology 34, 876-881, 2016.
Kimura et al., Dev. Growth Differ., 39(3):257-65, 1997.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, N Y, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
LaPointe et al., Hypertension, 27(3):715-22, 1996
LaPointe et al., J. Biol. Chem., 263(19):9075-8, 1988.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Nature, 294:228, 1981.
Levinson et al., Nature, 295:79, 1982.
Levrero et al., Gene, 101:195-202, 1991.
Lin et al., Mol. Cell. Biol., 10:850, 1990.
Long et al., Science 345: 1184-1188, 2014.
Long et al., Science 351, 400-403, 2016.
Pinello et al., Nature Biotechnol. 34, 695-697, 2016.
Long et al., JAMA Neurol., 3388, 2016.
Long et al., Science 351, 400-403, 2016.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Majors and Varmus, Proc. Natl. Acad. Sci. USA, 80:5866, 1983.
Mali et al., Science 339, 823-826, 2013a.
Mali et al., Nat Methods 10, 957-963, 2013b.
Mali et al., Nat. Biotechnol. 31:833-838, 2013c.
Mann et al., Cell, 33:153-159, 1983.
Maresca et al., Genome Research 23, 539-546, 2013.
Markowitz et al., J. Virol., 62:1120-1124, 1988.
McNeall et al., Gene, 76:81, 1989.
Miksicek et al., Cell, 46:203, 1986.
Millay et al., Nat. Med. 14, 442-447, 2008.
Mojica et al., J. Mol. Evol. 60, 174-182, 2005.
Mordacq and Linzer, Genes and Dev., 3:760, 1989.
Moreau et al., Nucl. Acids Res., 9:6047, 1981.
Moss et al., J. Gen. Physiol., 108(6):473-84, 1996.
Muesing et al., Cell, 48:691, 1987.
Nelson et al., Science 351, 403-407, 2016.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Ondek et al., EMBO J., 6:1017, 1987.
Ornitz et al., Mol. Cell. Biol., 7:3466, 1987.
Padgett, R. A., Trends Genet. 28, 147-154, 2012.
Palmiter et al., Cell, 29:701, 1982a.
Palmiter et al., Nature, 300:611, 1982b.
Paskind et al., Virology, 67:242-248, 1975.
Pech et al., Mol. Cell. Biol., 9:396, 1989.
Perales et al., Proc. Natl. Acad. Sci. USA, 91(9):4086-4090, 1994.
Perez-Stable and Constantin, Mol. Cell. Biol., 10:1116, 1990.
Picard et al., Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotech. Techniques, 9:169-174, 1995.
Ragot et al., Nature, 361:647-650, 1993.
Ran et al., Nature 520, 186-191, 2015.
Redondo et al., Science, 247:1225, 1990.
Reisman and Rotter, Mol. Cell. Biol., 9:3571, 1989.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Stoneham: Butterworth, 467-492, 1988.
Ripe et al., Mol. Cell. Biol., 9:2224, 1989.
Rippe et al., Mol. Cell Biol., 10:689-695, 1990.
Riffling et al., Nuc. Acids Res., 17:1619, 1989.
Rosen et al., Cell, 41:813, 1988.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Sambrook and Russell, Molecular Cloning: A Laboratory Manual 3^{rd} Ed., Cold Spring Harbor Laboratory Press, 2001.
Sakai et al., Genes and Dev., 2:1144, 1988.
Satake et al., J. Virology, 62:970, 1988.
Schaffner et al., J. Mol. Biol., 201:81, 1988.
Searle et al., Mol. Cell. Biol., 5:1480, 1985.
Sharp et al., Cell, 59:229, 1989.
Shaul and Ben-Levy, EMBO J., 6:1913, 1987.
Sherman et al., Mol. Cell. Biol., 9:50, 1989.
Sleigh et al., J. EMBO, 4:3831, 1985.
Shimizu-Motohashi et al., Am J Transl Res 8, 2471-89, 2016.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. 1, 248:1, 1987.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Tabebordbar et al., Science 351, 407-411, 2016.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Thiesen et al., J. Virology, 62:614, 1988.
Top et al., J. Infect. Dis., 124:155-160, 1971.
Tóth et al., Biology Direct, 1-14, 2016.
Tronche et al., Mol. Biol. Med., 7:173, 1990.
Trudel and Constantini, Genes and Dev. 6:954, 1987.
Tsai et al., Nature Biotechnology 34, 882-887, 2016.
Tur-Kaspa et al., Mol. Cell Biol., 6:716-718, 1986.
Tyndell et al., Nuc. Acids. Res., 9:6231, 1981.
Vannice and Levinson, J. Virology, 62:1305, 1988.
Varmus et al., Cell, 25:23-36, 1981.
Vasseur et al., Proc Natl. Acad. Sci. USA., 77:1068, 1980.
Wagner et al., Proc. Natl. Acad. Sci. USA, 87(9):3410-3414, 1990.
Wang and Calame, Cell, 47:241, 1986.
Wang et al., Cell, 153:910-910, 2013.
Weber et al., Cell, 36:983, 1984.
Weinberger et al. Mol. Cell. Biol., 8:988, 1984.
Winoto et al., Cell, 59:649, 1989.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Wu et al., Cell Stem Cell 13, 659-662, 2013.
Wu et al., Nat Biotechnol 32, 670-676, 2014.
Xu et al., Mol Ther 24, 564-569, 2016.
Yamauchi-Takihara et al., Proc. Natl. Acad. Sci. USA, 86(10):3504-3508, 1989.
Yang et al., Proc. Natl. Acad. Sci. USA, 87:9568-9572, 1990.
Yin et al., Nat Biotechnol 32, 551-553, 2014.
Yin et al., Physiol Rev 93, 23-67, 2013.
Young et al., Cell Stem Cell 18, 533-540, 2016.
Yutzey et al. Mol. Cell. Biol., 9:1397, 1989.
Zechner et al., Cell Metabolism 12, 633-642, 2010.
Zelenin et al., FEBS Lett., 280:94-96, 1991.
Zetsche et al., Cell 163, 759-771, 2015.
Ziober and Kramer, J. Bio. Chem., 271(37):22915-22922, 1996.

1. A composition comprising a sequence encoding a Cas9 polypeptide, a sequence encoding a first guide RNA (gRNA) targeting a first genomic target sequence, and a sequence encoding a second gRNA targeting a second genomic target sequence, wherein the first and second genomic target sequences each comprise an intronic sequence surrounding an exon of the murine dystrophin gene.

2. The composition of claim 1, wherein the exon comprises exon 50 of the murine dystrophin gene.

3. The composition of claim 1, wherein the sequence encoding a Cas9 polypeptide is isolated or derived from a sequence encoding a S. aureus Cas9 polypeptide.

4. The composition of claim 1, wherein at least one of the sequence encoding the Cas9 polypeptide, the sequence encoding the first gRNA, or the sequence encoding the second gRNA comprises an RNA sequence.

5. The composition of claim 4, wherein the RNA sequence comprises an mRNA sequence.

6. The composition of claim 4, wherein the RNA sequence comprises at least one chemically-modified nucleotide.

7. The composition of claim 1, wherein at least one of the sequence encoding the Cas9 polypeptide, the sequence encoding the first gRNA, or the sequence encoding the second gRNA comprises a DNA sequence.

8. The composition of claim 1, wherein a first vector comprises the sequence encoding the Cas9 polypeptide and a second vector comprises at least one of the sequence encoding the first gRNA or the sequence encoding the second gRNA.

9. The composition of claim 8, wherein the first vector or the sequence encoding the Cas9 polypeptide further comprises a first polyA sequence.

10. The composition of claim 8, wherein the second vector or the sequence encoding the first gRNA or the sequence encoding the second gRNA encodes a second polyA sequence.

11. The composition of claim 8, wherein the first vector or the sequence encoding the Cas9 polypeptide further comprises a first promoter sequence.

12. The composition of claim 8, wherein the second vector or the sequence encoding the first gRNA or the sequence encoding the second gRNA comprises a second promoter sequence.

13. The composition of claim 11, wherein the first promoter sequence and the second promoter sequence are identical.

14. The composition of claim 11, wherein the first promoter sequence and the second promoter sequence are not identical.

15. The composition of claim 11, wherein the first promoter sequence or the second promoter sequence comprises a CK8 promoter sequence.

16. The composition of claim 11, wherein the first promoter sequence or the second promoter sequence comprises a CK8e promoter sequence.

17. The composition of claim 11, wherein the first promoter sequence or the second promoter sequence comprises a constitutive promoter.

18. The composition of claim 11, wherein the first promoter sequence or the second promoter sequences comprises an inducible promoter.

19. The composition of claim 1, wherein one vector comprises the sequence encoding the Cas9 polypeptide, the sequence encoding the first gRNA and the sequence encoding the second gRNA.

20. The composition of claim 19, wherein the vector further comprises a polyA sequence.

21. The composition of claim 20, wherein the vector further comprises a promoter sequence.

22. The composition of claim 21, wherein the promoter sequence comprises a constitutive promoter.

23. The composition of claim 21, wherein the promoter sequence comprises an inducible promoter.

24. The composition of claim 21, wherein the promoter sequence comprises a CK8 promoter sequence.

25. The composition of claim 21, wherein the promoter sequence comprises a CK8e promoter sequence.

26. The composition of claim 1, wherein the composition comprises a sequence codon optimized for expression in a mammalian cell.

27. The composition of claim 1, wherein the composition comprises a sequence codon optimized for expression in a human cell or a mouse cell.

28. The composition of claim 27, wherein the sequence encoding the Cas9 polypeptide is codon optimized for expression in human cells or mouse cells.

29. The composition of claim 8, wherein at least one of the first vector and the second vector is a non-viral vector.

30. The composition of claim 29, wherein the non-viral vector is a plasmid.

31. The composition of claim 29, wherein a liposome or nanoparticle comprises the non-viral vector.

32. The composition of claim 8, wherein at least one of the first vector and the second vector is a viral vector.

33. The composition of claim 18, wherein the vector is a viral vector.

34. The composition of claim 32, wherein the viral vector is an adeno-associated viral (AAV) vector.

35. The composition of claim 34, wherein the AAV vector is replication-defective or conditionally replication defective.

36. The composition of claim 34, wherein the AAV vector is a recombinant AAV vector.

37. The composition of claim 34, wherein the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

38. The composition of claim 1, further comprising a pharmaceutically carrier.

39. A cell comprising the composition of claim 1.

40. The cell of claim 39, wherein the cell is a murine cell.

41. The cell of claim 39, wherein the cell is an oocyte.

42. A composition comprising the cell of claim 39.

43. A genetically engineered mouse comprising the cell of claim 39.

44. A method of creating a genetically engineered mouse comprising contacting the cell of claim 39 with a mouse.

45. A method of creating a genetically engineered mouse comprising contacting a cell of the mouse with a composition of claim 1.

46. A genetically engineered mouse generated by the method of claim 44.

47. A genetically engineered mouse, wherein the genome of the mouse comprises a deletion of exon 50 of the dystrophin gene resulting in an out of frame shift and a premature stop codon in exon 51 of the dystrophin gene.

48. The genetically engineered mouse of claim 47, further comprising a reporter gene located downstream of and in frame with exon 79 of the dystrophin gene, and upstream of a dystrophin 3′-UTR, wherein the reporter gene is expressed when exon 79 is translated in frame with exon 49.

49. The genetically engineered mouse of claim 48, wherein the reporter gene is luciferase.

50. The genetically engineered mouse of claim 47, further comprising a protease coding sequence upstream of and in frame with the reporter gene, and downstream of and in frame with exon 79.

51. The genetically engineered mouse of claim 50, wherein the protease is autocatalytic.

52. The genetically engineered mouse of claim 50, wherein the protease is 2A protease.

53. The genetically engineered mouse of claim 47, wherein the mouse is heterozygous for the deletion.

54. The genetically engineered mouse of claim 47, wherein the mouse is homozygous for the deletion.

55. The genetically engineered mouse of claim 47, wherein the mouse exhibits increased creatine kinase levels compared to a wildtype mouse.

56. The genetically engineered mouse of claim 47, wherein the mouse does not exhibit detectable dystrophin protein in heart or skeletal muscle.

57. A method of producing the genetically engineered mouse of any claim 47 comprising:
(a) contacting a fertilized oocyte with CRISPR/Cas9 elements and two single guide RNA (sgRNA) targeting sequences flanking exon 50 of the dystrophin gene, thereby creating a modified oocyte, wherein deletion of exon 50 by CRISPR/Cas9 results in an out of frame shift and a premature stop codon in exon 51 of the dystrophin gene;
(b) transferring the modified oocyte into a recipient female.

58. The method of claim 57, wherein the oocyte comprises a dystrophin gene having a reporter gene located downstream of and in frame with exon 79 of the dystrophin gene, and upstream of a dystrophin 3′-UTR, wherein the reporter gene is expressed when exon 79 is translated in frame with exon 49.

59. The method of claim 58, wherein the reporter gene is luciferase.

60. The method of claim 57, further comprising a protease coding sequence upstream of and in frame with the reporter gene, and downstream of and in frame with exon 79.

61. The method of claim 60, wherein the protease is autocatalytic.

62. The method of claim 60 or 61, wherein the protease is 2A protease.

63. The method of claim 57, wherein the mouse is heterozygous for the deletion.

64. The method of claim 57, wherein the mouse is homozygous for the deletion.

65. The method of claim 57, wherein the mouse exhibits increased creatine kinase levels compared to a wildtype mouse.

66. The method of claim 57, wherein the mouse does not exhibit detectable dystrophin protein in heart or skeletal muscle.

67. An isolated cell obtained from the genetically engineered mouse of claim 46.

68. The cell of claim 67, further comprising a reporter gene located downstream of and in frame with exon 79 of the dystrophin gene, and upstream of a dystrophin 3′-UTR, wherein the reporter gene is expressed when exon 79 is translated in frame with exon 49, in particular wherein the reporter is luciferase.

69. The cell of claim 66, further comprising a protease coding sequence upstream of and in frame with the reporter gene, and downstream of and in frame with exon 79.

70. The cell of claim 69, wherein the protease is autocatalytic.

71. The cell of claim 69, wherein the protease is 2A protease.

72. The cell of claim 69, wherein the cell is heterozygous for the deletion.

73. The cell of claim 67, wherein the cell is homozygous for the deletion.

74. A genetically engineered mouse produced by a method comprising the steps of:
(a) contacting a fertilized oocyte with CRISPR/Cas9 elements and two single guide RNA (sgRNA) targeting sequences flanking exon 50 of the dystrophin gene, thereby creating a modified oocyte, wherein deletion of exon 50 by CRISPR/Cas9 results in an out of frame shift and a premature stop codon in exon 51 of the dystrophin gene;
(b) transferring the modified oocyte into a recipient female.

75. A method of screening a candidate substance for DMD exon-skipping activity comprising:
(a) contacting a mouse according to claim 43 with the candidate substance; and
(b) assessing in frame transcription and/or translation of exon 79 of the dystrophin gene,
wherein the presence of in frame transcription and/or translation of exon 79 indicates the candidate substance exhibits exon-skipping activity.

76. A method of producing the genetically engineered mouse of claim 47 comprising:
(a) contacting a fertilized oocyte with CRISPR/Cpf1 elements and two single guide RNA (sgRNA) targeting sequences flanking exon 50 of the dystrophin gene, thereby creating a modified oocyte, wherein deletion of exon 50 by CRISPR/Cpf1 results in an out of frame shift and a premature stop codon in exon 51 of the dystrophin gene;
(b) transferring the modified oocyte into a recipient female.

77. A genetically engineered mouse produced by a method comprising the steps of:
(a) contacting a fertilized oocyte with CRISPR/Cpf1 elements and two single guide RNA (sgRNA) targeting sequences flanking exon 50 of the dystrophin gene, thereby creating a modified oocyte, wherein deletion of exon 50 by CRISPR/Cpf1 results in an out of frame shift and a premature stop codon in exon 51 of the dystrophin gene;
(b) transferring the modified oocyte into a recipient female.


SEQUENCE LISTING (788 sequences)

SEQ ID NO: 1 - DNA, 26 bp, Artificial Sequence
  source: /note=“Description of Artificial Sequence: Synthetic primer”
caccgaaatgatgagtgaagttatat

SEQ ID NO: 2 - DNA, 26 bp, Artificial Sequence
  source: /note=“Description of Artificial Sequence: Synthetic primer”
aaacatataacttcactcatcatttc

SEQ ID NO: 3 - DNA, 25 bp, Artificial Sequence
  source: /note=“Description of Artificial Sequence: Synthetic primer”
caccggtttgttcaaaagcgtggct

SEQ ID NO: 4 - DNA, 24 bp, Artificial Sequence
  source: /note=“Description of Artificial Sequence: Synthetic primer”
aaacagccacgcttttgaacaaac

SEQ ID NO: 5 - DNA, 24 bp, Artificial Sequence
  source: /note=“Description of Artificial Sequence: Synthetic primer”
caccggacacaatgtaggaagcct

SEQ ID NO: 6 - DNA, 24 bp, Artificial Sequence
  source: /note=“Description of Artificial Sequence: Synthetic primer”
aaacaggcttcctacattgtgtcc

SEQ ID NO: 7 - DNA, 45 bp, Artificial Sequence
  source: /note=“Description of Artificial Sequence: Synthetic primer”
gaattgtaatacgactcactataggaatgatgagtgaagttatat

SEQ ID NO: 8 - DNA, 45 bp, Artificial Sequence
  source: /note=“Description of Artificial Sequence: Synthetic primer”
gaattgtaatacgactcactatagggtttgttcaaaagcgtggct

SEQ ID NO: 9 - DNA, 22 bp, Artificial Sequence
  source: /note=“Description of Artificial Sequence: Synthetic primer”
aaaagcaccgactcggtgccac

SEQ ID NO: 10 - DNA, 24 bp, Artificial Sequence
  source: /note=“Description of Artificial Sequence: Synthetic primer”
aaacagccacgcttttgaacaaac

SEQ ID NO: 11 - DNA, 40 bp, Artificial Sequence
  source: /note=“Description of Artificial Sequence: Synthetic primer”
gaattgtaatacgactcactggacacaatgtaggaagcct

SEQ ID NO: 12 - DNA, 22 bp, Artificial Sequence
  source: /note=“Description of Artificial Sequence: Synthetic primer”
aaaagcaccgactcggtgccac

SEQ ID NO: 13 - DNA, 25 bp, Artificial Sequence
  source: /note=“Description of Artificial Sequence: Synthetic primer”
ggattgactgaaatgatggccaagg

SEQ ID NO: 14 - DNA, 24 bp, Artificial Sequence
  source: /note=“Description of Artificial Sequence: Synthetic primer”
ctgccacgattactctgcttccag

SEQ ID NO: 15 - DNA, 20 bp, Artificial Sequence
  source: /note=“Description of Artificial Sequence: Synthetic primer”
agcaggcagagaaggtggta

SEQ ID NO: 16 - DNA, 22 bp, Artificial Sequence
  source: /note=“Description of Artificial Sequence: Synthetic primer”
gggcgtatctcttcatagcctt

SEQ ID NO: 17 - DNA, 19 bp, Artificial Sequence
  source: /note=“Description of Artificial Sequence: Synthetic primer”
gcgtgtgtgtttgtttagg

SEQ ID NO: 18-33 - skipped (16 entries)

SEQ ID NO: 34 - DNA, 57 bp, Artificial Sequence
  source: /note=“Description of Artificial Sequence: Synthetic primer”
caccgtaatttctactaagtgtagataggctctgcaaagttctttgaaagttttttt

SEQ ID NO: 35 - DNA, 57 bp, Artificial Sequence
  source: /note=“Description of Artificial Sequence: Synthetic primer”
aaacaaaaaaactttcaaagaactttgcagagcctatctacacttagtagaaattac

SEQ ID NO: 36 - DNA, 57 bp, Artificial Sequence
  source: /note=“Description of Artificial Sequence: Synthetic primer”
caccgtaatttctactaagtgtagataaagagcaacaaaatggcttcaacttttttt

SEQ ID NO: 37 - DNA, 57 bp, Artificial Sequence
  source: /note=“Description of Artificial Sequence: Synthetic primer”
aaacaaaaaaagttgaagccattttgttgctctttatctacacttagtagaaattac

SEQ ID NO: 38 - DNA, 57 bp, Artificial Sequence
  source: /note=“Description of Artificial Sequence: Synthetic primer”
caccgtaatttctactaagtgtagataaagagcaataaaatggcttcaacttttttt

SEQ ID NO: 39 - DNA, 57 bp, Artificial Sequence
  source: /note=“Description of Artificial Sequence: Synthetic primer”
aaacaaaaaaagttgaagccattttattgctctttatctacacttagtagaaattac

SEQ ID NO: 40 - DNA, 57 bp, Artificial Sequence
  source: /note=“Description of Artificial Sequence: Synthetic primer”
caccgtaatttctactaagtgtagataaagaactttgcagagcctcaaaattttttt

SEQ ID NO: 41 - DNA, 57 bp, Artificial Sequence
  source: /note=“Description of Artificial Sequence: Synthetic primer”
aaacaaaaaaattttgaggctctgcaaagttctttatctacacttagtagaaattac

SEQ ID NO: 42 - DNA, 57 bp, Artificial Sequence
  source: /note=“Description of Artificial Sequence: Synthetic primer”
caccgtaatttctactaagtgtagatctgaatatctatgcattaataactttttttt

SEQ ID NO: 43 - DNA, 57 bp, Artificial Sequence
  source: /note=“Description of Artificial Sequence: Synthetic primer”
aaacaaaaaaaagttattaatgcatagatattcagatctacacttagtagaaattac

SEQ ID NO: 44 - DNA, 57 bp, Artificial Sequence
  source: /note=“Description of Artificial Sequence: Synthetic primer”
caccgtaatttctactaagtgtagattattatattacagggcatattatattttttt

SEQ ID NO: 45 - DNA, 57 bp, Artificial Sequence
  source: /note=“Description of Artificial Sequence: Synthetic primer”
aaacaaaaaaatataatatgccctgtaatataataatctacacttagtagaaattac

SEQ ID NO: 46 - DNA, 57 bp, Artificial Sequence
  source: /note=“Description of Artificial Sequence: Synthetic primer”
caccgtaatttctactaagtgtagataggtaagccgaggtttggcctttattttttt

SEQ ID NO: 47 - DNA, 57 bp, Artificial Sequence
  source: /note=“Description of Artificial Sequence: Synthetic primer”
aaacaaaaaaataaaggccaaacctcggcttacctatctacacttagtagaaattac

SEQ ID NO: 48 - DNA, 57 bp, Artificial Sequence
  source: /note=“Description of Artificial Sequence: Synthetic primer”
caccgtaatttctactaagtgtagatcccagagtccttcaaagatattgattttttt

SEQ ID NO: 49 - DNA, 57 bp, Artificial Sequence
  source: /note=“Description of Artificial Sequence: Synthetic primer”
aaacaaaaaaatcaatatctttgaaggactctgggatctacacttagtagaaattac

SEQ ID NO: 50 - DNA, 47 bp, Artificial Sequence
  source: /note=“Description of Artificial Sequence: Synthetic primer”
gaattgtaatacgactcactatagggtaatttctactaagtgtagat

SEQ ID NO: 51 - DNA, 45 bp, Artificial Sequence
  source: /note=“Description of Artificial Sequence: Synthetic primer”
ctttcaaagaactttgcagagcctatctacacttagtagaaatta

SEQ ID NO: 52 - DNA, 45 bp, Artificial Sequence
  source: /note=“Description of Artificial Sequence: Synthetic primer”
gttgaagccattttattgctctttatctacacttagtagaaatta

SEQ ID NO: 53 - DNA, 45 bp, Artificial Sequence
  source: /note=“Description of Artificial Sequence: Synthetic primer”
ttttgaggctctgcaaagttctttatctacacttagtagaaatta

SEQ ID NO: 54 - DNA, 57 bp, Artificial Sequence
  source: /note=“Description of Artificial Sequence: Synthetic primer”
tataatatgccctgtaatataataatctacacttagtagaaattaccctatagtgag

SEQ ID NO: 55 - DNA, 57 bp, Artificial Sequence
  source: /note=“Description of Artificial Sequence: Synthetic primer”
tcaatatctttgaaggactctgggatctacacttagtagaaattaccctatagtgag

SEQ ID NO: 56 - DNA, 27 bp, Artificial Sequence
  source: /note=“Description of Artificial Sequence: Synthetic primer”
gagaaacttctgtgatgtgaggacata

SEQ ID NO: 57 - DNA, 22 bp, Artificial Sequence
  source: /note=“Description of Artificial Sequence: Synthetic primer”
caaacctcggcttacctgaaat

SEQ ID NO: 58 - DNA, 27 bp, Artificial Sequence
  source: /note=“Description of Artificial Sequence: Synthetic primer”
caatatctttgaaggactctgggtaaa

SEQ ID NO: 59 - DNA, 27 bp, Artificial Sequence
  source: /note=“Description of Artificial Sequence: Synthetic primer”
aattaatagaagtcaatgtagggaagg

SEQ ID NO: 60-382 - skipped (323 entries)

SEQ ID NO: 383 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 384-441 - skipped (58 entries)

SEQ ID NO: 442 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 443 - protein, 1228 aa, Lachnospiraceae bacterium ND2006
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 444-447 - skipped (4 entries)

SEQ ID NO: 448 - RNA, 24 nt, Artificial Sequence
  source: /note=“Description of Artificial Sequence: Synthetic oligonucleotide”
aaaaaggaaaaaagaagaaaaaga

SEQ ID NO: 449 - RNA, 24 nt, Artificial Sequence
  source: /note=“Description of Artificial Sequence: Synthetic oligonucleotide”
caaaaaggaaaaaagaagaaaaag

SEQ ID NO: 450 - RNA, 24 nt, Artificial Sequence
  source: /note=“Description of Artificial Sequence: Synthetic oligonucleotide”
gcaaaaaggaaaaaagaagaaaaa

SEQ ID NO: 451 - RNA, 24 nt, Artificial Sequence
  source: /note=“Description of Artificial Sequence: Synthetic oligonucleotide”
uuuugcaaaaaggaaaaaagaaga

SEQ ID NO: 452 - RNA, 24 nt, Artificial Sequence
  source: /note=“Description of Artificial Sequence: Synthetic oligonucleotide”
uuuuugcaaaaaggaaaaaagaag

SEQ ID NO: 453 - RNA, 24 nt, Artificial Sequence
  source: /note=“Description of Artificial Sequence: Synthetic oligonucleotide”
guuuuugcaaaaaggaaaaaagaa

SEQ ID NO: 454 - RNA, 24 nt, Artificial Sequence
  source: /note=“Description of Artificial Sequence: Synthetic oligonucleotide”
auuuuggguuuuugcaaaaaggaa

SEQ ID NO: 455 - RNA, 24 nt, Artificial Sequence
  source: /note=“Description of Artificial Sequence: Synthetic oligonucleotide”
uauuuuggguuuuugcaaaaagga

SEQ ID NO: 456 - RNA, 24 nt, Artificial Sequence
  source: /note=“Description of Artificial Sequence: Synthetic oligonucleotide”
auauuuuggguuuuugcaaaaagg

SEQ ID NO: 457 - RNA, 24 nt, Artificial Sequence
  source: /note=“Description of Artificial Sequence: Synthetic oligonucleotide”
aauauuuuggguuuuugcaaaaag

SEQ ID NO: 458 - RNA, 24 nt, Artificial Sequence
  source: /note=“Description of Artificial Sequence: Synthetic oligonucleotide”
gcuaaaauauuuuggguuuuugca

SEQ ID NO: 459 - RNA, 24 nt, Artificial Sequence
  source: /note=“Description of Artificial Sequence: Synthetic oligonucleotide”
agcuaaaauauuuuggguuuuugc

SEQ ID NO: 460 - RNA, 24 nt, Artificial Sequence
  source: /note=“Description of Artificial Sequence: Synthetic oligonucleotide”
gagcuaaaauauuuuggguuuuug

SEQ ID NO: 461 - RNA, 24 nt, Artificial Sequence
  source: /note=“Description of Artificial Sequence: Synthetic oligonucleotide”
agaguaacagucugaguaggagcu

SEQ ID NO: 462 - RNA, 24 nt, Artificial Sequence
  source: /note=“Description of Artificial Sequence: Synthetic oligonucleotide”
cagaguaacagucugaguaggagc

SEQ ID NO: 463 - RNA, 24 nt, Artificial Sequence
  source: /note=“Description of Artificial Sequence: Synthetic oligonucleotide”
gugacacaaccugugguuacuaag

SEQ ID NO: 464 - RNA, 23 nt, Artificial Sequence
  source: /note=“Description of Artificial Sequence: Synthetic oligonucleotide”
gguuacuaaggaaacugccaucu

SEQ ID NO: 465 - RNA, 24 nt, Artificial Sequence
  source: /note=“Description of Artificial Sequence: Synthetic oligonucleotide”
aaggaaacugccaucuccaaacua

SEQ ID NO: 466 - RNA, 24 nt, Artificial Sequence
  source: /note=“Description of Artificial Sequence: Synthetic oligonucleotide”
aucaucaagcagaagguaugagaa

SEQ ID NO: 467 - RNA, 24 nt, Artificial Sequence
  source: /note=“Description of Artificial Sequence: Synthetic oligonucleotide”
agcagaagguaugagaaaaaauga

SEQ ID NO: 468 - RNA, 24 nt, Artificial Sequence
  source: /note=“Description of Artificial Sequence: Synthetic oligonucleotide”
gcagaagguaugagaaaaaaugau

SEQ ID NO: 469 - RNA, 24 nt, Artificial Sequence
  source: /note=“Description of Artificial Sequence: Synthetic oligonucleotide”
uaaaaguuggcagaaguuuuucuu

SEQ ID NO: 470 - RNA, 24 nt, Artificial Sequence
  source: /note=“Description of Artificial Sequence: Synthetic oligonucleotide”
aaaaguuggcagaaguuuuucuuu

SEQ ID NO: 471 - RNA, 24 nt, Artificial Sequence
  source: /note=“Description of Artificial Sequence: Synthetic oligonucleotide”
gguggaaaaucuucauuuuaaaga

SEQ ID NO: 472 - RNA, 24 nt, Artificial Sequence
  source: /note=“Description of Artificial Sequence: Synthetic oligonucleotide”
ugguggaaaaucuucauuuuaaag

SEQ ID NO: 473 - RNA, 24 nt, Artificial Sequence
  source: /note=“Description of Artificial Sequence: Synthetic oligonucleotide”
uugguggaaaaucuucauuuuaaa

SEQ ID NO: 474 - RNA, 24 nt, Artificial Sequence
  source: /note=“Description of Artificial Sequence: Synthetic oligonucleotide”
gugauugguggaaaaucuucauuu

SEQ ID NO: 475 - RNA, 24 nt, Artificial Sequence
  source: /note=“Description of Artificial Sequence: Synthetic oligonucleotide”
cuaggagaguaaagugauuggugg

SEQ ID NO: 476 - RNA, 24 nt, Artificial Sequence
  source: /note=“Description of Artificial Sequence: Synthetic oligonucleotide”
ucuaggagaguaaagugauuggug

SEQ ID NO: 477 - RNA, 23 nt, Artificial Sequence
  source: /note=“Description of Artificial Sequence: Synthetic oligonucleotide”
cuggugggaaauggucuaggaga

SEQ ID NO: 478 - RNA, 24 nt, Artificial Sequence
  source: /note=“Description of Artificial Sequence: Synthetic oligonucleotide”
guagcacacuguuuaaucuuuucu

SEQ ID NO: 479 - RNA, 24 nt, Artificial Sequence
  source: /note=“Description of Artificial Sequence: Synthetic oligonucleotide”
cacacuguuuaaucuuuucucaaa

SEQ ID NO: 480 - RNA, 24 nt, Artificial Sequence
  source: /note=“Description of Artificial Sequence: Synthetic oligonucleotide”
acacuguuuaaucuuuucucaaau

SEQ ID NO: 481 - RNA, 24 nt, Artificial Sequence
  source: /note=“Description of Artificial Sequence: Synthetic oligonucleotide”
cacuguuuaaucuuuucucaaaua

SEQ ID NO: 482 - RNA, 24 nt, Artificial Sequence
  source: /note=“Description of Artificial Sequence: Synthetic oligonucleotide”
augucuuuuuauuugagaaaagau

SEQ ID NO: 483 - RNA, 24 nt, Artificial Sequence
  source: /note=“Description of Artificial Sequence: Synthetic oligonucleotide”
aagccccaugucuuuuuauuugag

SEQ ID NO: 484 - RNA, 24 nt, Artificial Sequence
  source: /note=“Description of Artificial Sequence: Synthetic oligonucleotide”
gaagccccaugucuuuuuauuuga

SEQ ID NO: 485 - RNA, 24 nt, Artificial Sequence
  source: /note=“Description of Artificial Sequence: Synthetic oligonucleotide”
guaagauaccaaaaaggcaaaaca

SEQ ID NO: 486 - RNA, 24 nt, Artificial Sequence
  source: /note=“Description of Artificial Sequence: Synthetic oligonucleotide”
uguaagauaccaaaaaggcaaaac

SEQ ID NO: 487 - RNA, 24 nt, Artificial Sequence
  source: /note=“Description of Artificial Sequence: Synthetic oligonucleotide”
cuguaagauaccaaaaaggcaaaa

SEQ ID NO: 488 - RNA, 24 nt, Artificial Sequence
  source: /note=“Description of Artificial Sequence: Synthetic oligonucleotide”
guuccuguaagauaccaaaaaggc

SEQ ID NO: 489 - RNA, 24 nt, Artificial Sequence
  source: /note=“Description of Artificial Sequence: Synthetic oligonucleotide”
aguuccuguaagauaccaaaaagg

SEQ ID NO: 490 - RNA, 24 nt, Artificial Sequence
  source: /note=“Description of Artificial Sequence: Synthetic oligonucleotide”
uccuggaguuccuguaagauacca

SEQ ID NO: 491 - RNA, 24 nt, Artificial Sequence
  source: /note=“Description of Artificial Sequence: Synthetic oligonucleotide”
auccuggaguuccuguaagauacc

SEQ ID NO: 492 - RNA, 24 nt, Artificial Sequence
  source: /note=“Description of Artificial Sequence: Synthetic oligonucleotide”
gggaagaaauaauucagcaauccu

SEQ ID NO: 493 - RNA, 24 nt, Artificial Sequence
  source: /note=“Description of Artificial Sequence: Synthetic oligonucleotide”
ggaagaaauaauucagcaauccuc

SEQ ID NO: 494 - RNA, 24 nt, Artificial Sequence
  source: /note=“Description of Artificial Sequence: Synthetic oligonucleotide”
gaagaaauaauucagcaauccuca

SEQ ID NO: 495 - RNA, 24 nt, Artificial Sequence
  source: /note=“Description of Artificial Sequence: Synthetic oligonucleotide”
aaaacagaugccaguauucuacag

SEQ ID NO: 496 - RNA, 24 nt, Artificial Sequence
  source: /note=“Description of Artificial Sequence: Synthetic oligonucleotide”
aaacagaugccaguauucuacagg

SEQ ID NO: 497 - RNA, 24 nt, Artificial Sequence
  source: /note=“Description of Artificial Sequence: Synthetic oligonucleotide”
aacagaugccaguauucuacagga

SEQ ID NO: 498 - RNA, 24 nt, Artificial Sequence
  source: /note=“Description of Artificial Sequence: Synthetic oligonucleotide”
gaaucugcgguggcaggaggucug

SEQ ID NO: 499 - RNA, 24 nt, Artificial Sequence
  source: /note=“Description of Artificial Sequence: Synthetic oligonucleotide”
aggucugcaaacagcugucagaca

SEQ ID NO: 500 - RNA, 24 nt, Artificial Sequence
  source: /note=“Description of Artificial Sequence: Synthetic oligonucleotide”
ggucugcaaacagcugucagacag

SEQ ID NO: 501 - RNA, 24 nt, Artificial Sequence
  source: /note=“Description of Artificial Sequence: Synthetic oligonucleotide”
gucugcaaacagcugucagacaga

SEQ ID NO: 502 - RNA, 24 nt, Artificial Sequence
  source: /note=“Description of Artificial Sequence: Synthetic oligonucleotide”
ucugcaaacagcugucagacagaa

SEQ ID NO: 503 - RNA, 24 nt, Artificial Sequence
  source: /note=“Description of Artificial Sequence: Synthetic oligonucleotide”
uagggcgacagaucuaauaggaau

SEQ ID NO: 504 - RNA, 24 nt, Artificial Sequence
  source: /note=“Description of Artificial Sequence: Synthetic oligonucleotide”
agggcgacagaucuaauaggaaug

SEQ ID NO: 505 - RNA, 24 nt, Artificial Sequence
  source: /note=“Description of Artificial Sequence: Synthetic oligonucleotide”
uaaagaaagcuuaaaaagucugcu

SEQ ID NO: 506 - RNA, 24 nt, Artificial Sequence
  source: /note=“Description of Artificial Sequence: Synthetic oligonucleotide”
cuaaagaaagcuuaaaaagucugc

SEQ ID NO: 507 - RNA, 24 nt, Artificial Sequence
  source: /note=“Description of Artificial Sequence: Synthetic oligonucleotide”
aaauauucuucuaaagaaagcuua

SEQ ID NO: 508 - RNA, 24 nt, Artificial Sequence
  source: /note=“Description of Artificial Sequence: Synthetic oligonucleotide”
gaaauauucuucuaaagaaagcuu

SEQ ID NO: 509 - RNA, 24 nt, Artificial Sequence
  source: /note=“Description of Artificial Sequence: Synthetic oligonucleotide”
ugaaauauucuucuaaagaaagcu

SEQ ID NO: 510 - RNA, 24 nt, Artificial Sequence
  source: /note=“Description of Artificial Sequence: Synthetic oligonucleotide”
ucucucaugaaauauucuucuaaa

SEQ ID NO: 511 - RNA, 24 nt, Artificial Sequence
  source: /note=“Description of Artificial Sequence: Synthetic oligonucleotide”
auaaucucucaugaaauauucuuc

SEQ ID NO: 512 - RNA, 24 nt, Artificial Sequence
  source: /note=“Description of Artificial Sequence: Synthetic oligonucleotide”
gcguauauuuuuugguuauacuga

SEQ ID NO: 513 - RNA, 24 nt, Artificial Sequence
  source: /note=“Description of Artificial Sequence: Synthetic oligonucleotide”
ucaagaaaaauagauggauuaugu

SEQ ID NO: 514 - RNA, 24 nt, Artificial Sequence
  source: /note=“Description of Artificial Sequence: Synthetic oligonucleotide”
aucaagaaaaauagauggauuaug

SEQ ID NO: 515 - RNA, 24 nt, Artificial Sequence
  source: /note=“Description of Artificial Sequence: Synthetic oligonucleotide”
cagguaaaagcauauggaucaaga

SEQ ID NO: 516 - RNA, 24 nt, Artificial Sequence
  source: /note=“Description of Artificial Sequence: Synthetic oligonucleotide”
gcagguaaaagcauauggaucaag

SEQ ID NO: 517 - RNA, 24 nt, Artificial Sequence
  source: /note=“Description of Artificial Sequence: Synthetic oligonucleotide”
ugcagguaaaagcauauggaucaa

SEQ ID NO: 518 - RNA, 24 nt, Artificial Sequence
  source: /note=“Description of Artificial Sequence: Synthetic oligonucleotide”
caggcgauuugacagaucuguuga

SEQ ID NO: 519 - RNA, 24 nt, Artificial Sequence
  source: /note=“Description of Artificial Sequence: Synthetic oligonucleotide”
agaucugucaaaucgccugcaggu

SEQ ID NO: 520 - RNA, 24 nt, Artificial Sequence
  source: /note=“Description of Artificial Sequence: Synthetic oligonucleotide”
cagaucugucaaaucgccugcagg

SEQ ID NO: 521 - RNA, 24 nt, Artificial Sequence
  source: /note=“Description of Artificial Sequence: Synthetic oligonucleotide”
gccgccauuucucaacagaucugu

SEQ ID NO: 522 - RNA, 24 nt, Artificial Sequence
  source: /note=“Description of Artificial Sequence: Synthetic oligonucleotide”
aauggcggcguuuucauuaugaua

SEQ ID NO: 523 - RNA, 24 nt, Artificial Sequence
  source: /note=“Description of Artificial Sequence: Synthetic oligonucleotide”
auuaaauaucuuuauaucauaaug

SEQ ID NO: 524 - RNA, 24 nt, Artificial Sequence
  source: /note=“Description of Artificial Sequence: Synthetic oligonucleotide”
ugagaauugggaacaugcuaaaua

SEQ ID NO: 525 - RNA, 24 nt, Artificial Sequence
  source: /note=“Description of Artificial Sequence: Synthetic oligonucleotide”
gguaagucuuugauuuguuuuuuc

SEQ ID NO: 526 - RNA, 24 nt, Artificial Sequence
  source: /note=“Description of Artificial Sequence: Synthetic oligonucleotide”
aaauacaauuucgaaaaaacaaau

SEQ ID NO: 527 - RNA, 24 nt, Artificial Sequence
  source: /note=“Description of Artificial Sequence: Synthetic oligonucleotide”
aagauaaauacaauuucgaaaaaa

SEQ ID NO: 528 - RNA, 24 nt, Artificial Sequence
  source: /note=“Description of Artificial Sequence: Synthetic oligonucleotide”
gcugaagauaaauacaauuucgaa

SEQ ID NO: 529 - RNA, 24 nt, Artificial Sequence
  source: /note=“Description of Artificial Sequence: Synthetic oligonucleotide”
ugcugaagauaaauacaauuucga

SEQ ID NO: 530 - RNA, 24 nt, Artificial Sequence
  source: /note=“Description of Artificial Sequence: Synthetic oligonucleotide”
gugcugaagauaaauacaauuucg

SEQ ID NO: 531 - RNA, 24 nt, Artificial Sequence
  source: /note=“Description of Artificial Sequence: Synthetic oligonucleotide”
ugugcugaagauaaauacaauuuc

SEQ ID NO: 532 - RNA, 24 nt, Artificial Sequence
  source: /note=“Description of Artificial Sequence: Synthetic oligonucleotide”
gcacaucuggacucuuuaacuucu

SEQ ID NO: 533 - RNA, 24 nt, Artificial Sequence
  source: /note=“Description of Artificial Sequence: Synthetic oligonucleotide”
uaaagaguccagaugugcugaaga

SEQ ID NO: 534 - RNA, 24 nt, Artificial Sequence
  source: /note=“Description of Artificial Sequence: Synthetic oligonucleotide”
aagaucagguucugaagggugaug

SEQ ID NO: 535 - RNA, 24 nt, Artificial Sequence
  source: /note=“Description of Artificial Sequence: Synthetic oligonucleotide”
uucagaaccugaucuuuaagaagu

SEQ ID NO: 536 - RNA, 24 nt, Artificial Sequence
  source: /note=“Description of Artificial Sequence: Synthetic oligonucleotide”
aauauaaugaugacaacaacaguc

SEQ ID NO: 537 - RNA, 24 nt, Artificial Sequence
  source: /note=“Description of Artificial Sequence: Synthetic oligonucleotide”
uaauauaaugaugacaacaacagu

SEQ ID NO: 538 - RNA, 24 nt, Artificial Sequence
  source: /note=“Description of Artificial Sequence: Synthetic oligonucleotide”
uuuauuuuuccuuuuauucuaguu

SEQ ID NO: 539 - RNA, 24 nt, Artificial Sequence
  source: /note=“Description of Artificial Sequence: Synthetic oligonucleotide”
aaaggaaaaauaaauauauaguag

SEQ ID NO: 540 - RNA, 24 nt, Artificial Sequence
  source: /note=“Description of Artificial Sequence: Synthetic oligonucleotide”
uuucaacuagaauaaaaggaaaaa

SEQ ID NO: 541 - RNA, 24 nt, Artificial Sequence
  source: /note=“Description of Artificial Sequence: Synthetic oligonucleotide”
auucuuucaacuagaauaaaagga

SEQ ID NO: 542 - RNA, 24 nt, Artificial Sequence
  source: /note=“Description of Artificial Sequence: Synthetic oligonucleotide”
aauucuuucaacuagaauaaaagg

SEQ ID NO: 543 - RNA, 24 nt, Artificial Sequence
  source: /note=“Description of Artificial Sequence: Synthetic oligonucleotide”
gaauucuuucaacuagaauaaaag

SEQ ID NO: 544 - RNA, 24 nt, Artificial Sequence
  source: /note=“Description of Artificial Sequence: Synthetic oligonucleotide”
auucugaauucuuucaacuagaau

SEQ ID NO: 545 - RNA, 24 nt, Artificial Sequence
  source: /note=“Description of Artificial Sequence: Synthetic oligonucleotide”
gauucugaauucuuucaacuagaa

SEQ ID NO: 546 - RNA, 24 nt, Artificial Sequence
  source: /note=“Description of Artificial Sequence: Synthetic oligonucleotide”
cagaaccggaggcaacaguugaau

SEQ ID NO: 547 - RNA, 24 nt, Artificial Sequence
  source: /note=“Description of Artificial Sequence: Synthetic oligonucleotide”
ggaggcaacaguugaaugaaaugu

SEQ ID NO: 548 - RNA, 24 nt, Artificial Sequence
  source: /note=“Description of Artificial Sequence: Synthetic oligonucleotide”
uauacaguagaugcaauccaaaag

SEQ ID NO: 549 - RNA, 24 nt, Artificial Sequence
  source: /note=“Description of Artificial Sequence: Synthetic oligonucleotide”
gaugcaauccaaaagaaaaucaca

SEQ ID NO: 550 - RNA, 24 nt, Artificial Sequence
  source: /note=“Description of Artificial Sequence: Synthetic oligonucleotide”
aaucacagaaaccaagguuaguau

SEQ ID NO: 551 - RNA, 23 nt, Artificial Sequence
  source: /note=“Description of Artificial Sequence: Synthetic oligonucleotide”
agguuaguaucaaagauaccuuu

SEQ ID NO: 552 - RNA, 24 nt, Artificial Sequence
  source: /note=“Description of Artificial Sequence: Synthetic oligonucleotide”
gguuaguaucaaagauaccuuuuu

SEQ ID NO: 553 - RNA, 24 nt, Artificial Sequence
  source: /note=“Description of Artificial Sequence: Synthetic oligonucleotide”
aguaucaaagauaccuuuuuaaaa

SEQ ID NO: 554 - RNA, 24 nt, Artificial Sequence
  source: /note=“Description of Artificial Sequence: Synthetic oligonucleotide”
guaucaaagauaccuuuuuaaaau

SEQ ID NO: 555 - RNA, 24 nt, Artificial Sequence
  source: /note=“Description of Artificial Sequence: Synthetic oligonucleotide”
uguuugugucccaguuugcauuaa

SEQ ID NO: 556 - RNA, 24 nt, Artificial Sequence
  source: /note=“Description of Artificial Sequence: Synthetic oligonucleotide”
cugggacacaaacauggcaauuua

SEQ ID NO: 557 - RNA, 24 nt, Artificial Sequence
  source: /note=“Description of Artificial Sequence: Synthetic oligonucleotide”
acugggacacaaacauggcaauuu

SEQ ID NO: 558 - RNA, 24 nt, Artificial Sequence
  source: /note=“Description of Artificial Sequence: Synthetic oligonucleotide”
aacugggacacaaacauggcaauu

SEQ ID NO: 559 - RNA, 24 nt, Artificial Sequence
  source: /note=“Description of Artificial Sequence: Synthetic oligonucleotide”
uauuuguuaaugcaaacugggaca

SEQ ID NO: 560 - RNA, 24 nt, Artificial Sequence
  source: /note=“Description of Artificial Sequence: Synthetic oligonucleotide”
acaaauaguuugagaacuauguug

SEQ ID NO: 561 - RNA, 24 nt, Artificial Sequence
  source: /note=“Description of Artificial Sequence: Synthetic oligonucleotide”
caaauaguuugagaacuauguugg

SEQ ID NO: 562 - RNA, 24 nt, Artificial Sequence
  source: /note=“Description of Artificial Sequence: Synthetic oligonucleotide”
aaauaguuugagaacuauguugga

SEQ ID NO: 563 - RNA, 24 nt, Artificial Sequence
  source: /note=“Description of Artificial Sequence: Synthetic oligonucleotide”
auaguuugagaacuauguuggaaa

SEQ ID NO: 564 - RNA, 24 nt, Artificial Sequence
  source: /note=“Description of Artificial Sequence: Synthetic oligonucleotide”
uaguuugagaacuauguuggaaaa

SEQ ID NO: 565 - RNA, 24 nt, Artificial Sequence
  source: /note=“Description of Artificial Sequence: Synthetic oligonucleotide”
aguuugagaacuauguuggaaaaa

SEQ ID NO: 566 - RNA, 24 nt, Artificial Sequence
  source: /note=“Description of Artificial Sequence: Synthetic oligonucleotide”
uaguucucaaacuauuuguuaaug

SEQ ID NO: 567 - RNA, 24 nt, Artificial Sequence
  source: /note=“Description of Artificial Sequence: Synthetic oligonucleotide”
uauuuuuuuuuccaacauaguucu

SEQ ID NO: 568 - RNA, 24 nt, Artificial Sequence
  source: /note=“Description of Artificial Sequence: Synthetic oligonucleotide”
cuucuuucuccaggcuagaagaac

SEQ ID NO: 569 - RNA, 24 nt, Artificial Sequence
  source: /note=“Description of Artificial Sequence: Synthetic oligonucleotide”
cuucuagccuggagaaagaagaau

SEQ ID NO: 570 - RNA, 24 nt, Artificial Sequence
  source: /note=“Description of Artificial Sequence: Synthetic oligonucleotide”
ucuucuagccuggagaaagaagaa

SEQ ID NO: 571 - RNA, 24 nt, Artificial Sequence
  source: /note=“Description of Artificial Sequence: Synthetic oligonucleotide”
auucuuuuguucuucuagccugga

SEQ ID NO: 572 - RNA, 24 nt, Artificial Sequence
  source: /note=“Description of Artificial Sequence: Synthetic oligonucleotide”
caaaagaauaucuugucagaauuu

SEQ ID NO: 573 - RNA, 24 nt, Artificial Sequence
  source: /note=“Description of Artificial Sequence: Synthetic oligonucleotide”
cuggaaaagagcagcaacuaaaag

SEQ ID NO: 574 - RNA, 24 nt, Artificial Sequence
  source: /note=“Description of Artificial Sequence: Synthetic oligonucleotide”
caagucaagguaauuuuauuuucu

SEQ ID NO: 575 - RNA, 24 nt, Artificial Sequence
  source: /note=“Description of Artificial Sequence: Synthetic oligonucleotide”
caaaucccccagggccugcuugca

SEQ ID NO: 576 - RNA, 24 nt, Artificial Sequence
  source: /note=“Description of Artificial Sequence: Synthetic oligonucleotide”
aggcccugggggauuugagaaaau

SEQ ID NO: 577 - RNA, 24 nt, Artificial Sequence
  source: /note=“Description of Artificial Sequence: Synthetic oligonucleotide”
caggcccugggggauuugagaaaa

SEQ ID NO: 578 - RNA, 24 nt, Artificial Sequence
  source: /note=“Description of Artificial Sequence: Synthetic oligonucleotide”
caagcaggcccugggggauuugag

SEQ ID NO: 579 - RNA, 24 nt, Artificial Sequence
  source: /note=“Description of Artificial Sequence: Synthetic oligonucleotide”
gcaagcaggcccugggggauuuga

SEQ ID NO: 580 - RNA, 24 nt, Artificial Sequence
  source: /note=“Description of Artificial Sequence: Synthetic oligonucleotide”
gcagaaaaccaaugauugaauuaa

SEQ ID NO: 581 - RNA, 24 nt, Artificial Sequence
  source: /note=“Description of Artificial Sequence: Synthetic oligonucleotide”
ggcagaaaaccaaugauugaauua

SEQ ID NO: 582 - RNA, 24 nt, Artificial Sequence
  source: /note=“Description of Artificial Sequence: Synthetic oligonucleotide”
gggcagaaaaccaaugauugaauu

SEQ ID NO: 583 - RNA, 24 nt, Artificial Sequence
  source: /note=“Description of Artificial Sequence: Synthetic oligonucleotide”
ugggcagaaaaccaaugauugaau

SEQ ID NO: 584 - RNA, 24 nt, Artificial Sequence
  source: /note=“Description of Artificial Sequence: Synthetic oligonucleotide”
auuagguuauucauaguuccuugc

SEQ ID NO: 585 - RNA, 24 nt, Artificial Sequence
  source: /note=“Description of Artificial Sequence: Synthetic oligonucleotide”
aacuaugaauaaccuaaugggcag

SEQ ID NO: 586 - RNA, 24 nt, Artificial Sequence
  source: /note=“Description of Artificial Sequence: Synthetic oligonucleotide”
gaacuaugaauaaccuaaugggca

SEQ ID NO: 587 - RNA, 24 nt, Artificial Sequence
  source: /note=“Description of Artificial Sequence: Synthetic oligonucleotide”
uauuuccuguuaaauuguuuucua

SEQ ID NO: 588 - RNA, 24 nt, Artificial Sequence
  source: /note=“Description of Artificial Sequence: Synthetic oligonucleotide”
gguuuauagaaaacaauuuaacag

SEQ ID NO: 589 - RNA, 24 nt, Artificial Sequence
  source: /note=“Description of Artificial Sequence: Synthetic oligonucleotide”
auacaguaacaucuuuuuuauuuc

SEQ ID NO: 590 - RNA, 24 nt, Artificial Sequence
  source: /note=“Description of Artificial Sequence: Synthetic oligonucleotide”
uacaguaacaucuuuuuuauuucu

SEQ ID NO: 591 - RNA, 24 nt, Artificial Sequence
  source: /note=“Description of Artificial Sequence: Synthetic oligonucleotide”
auguuacuguauaaggguuuauag

SEQ ID NO: 592 - RNA, 24 nt, Artificial Sequence
  source: /note=“Description of Artificial Sequence: Synthetic oligonucleotide”
gauguuacuguauaaggguuuaua

SEQ ID NO: 593 - RNA, 24 nt, Artificial Sequence
  source: /note=“Description of Artificial Sequence: Synthetic oligonucleotide”
cagccaaaacacuuuuagaaauaa

SEQ ID NO: 594 - RNA, 24 nt, Artificial Sequence
  source: /note=“Description of Artificial Sequence: Synthetic oligonucleotide”
ccagccaaaacacuuuuagaaaua

SEQ ID NO: 595 - RNA, 24 nt, Artificial Sequence
  source: /note=“Description of Artificial Sequence: Synthetic oligonucleotide”
accagccaaaacacuuuuagaaau

SEQ ID NO: 596 - RNA, 24 nt, Artificial Sequence
  source: /note=“Description of Artificial Sequence: Synthetic oligonucleotide”
gaccagccaaaacacuuuuagaaa

SEQ ID NO: 597 - RNA, 24 nt, Artificial Sequence
  source: /note=“Description of Artificial Sequence: Synthetic oligonucleotide”
gugagaccagccaaaacacuuuua

SEQ ID NO: 598 - RNA, 24 nt, Artificial Sequence
  source: /note=“Description of Artificial Sequence: Synthetic oligonucleotide”
aauuguacuuuacuuuguauuaug

SEQ ID NO: 599 - RNA, 24 nt, Artificial Sequence
  source: /note=“Description of Artificial Sequence: Synthetic oligonucleotide”
auuguacuuuacuuuguauuaugu

SEQ ID NO: 600 - RNA, 24 nt, Artificial Sequence
  source: /note=“Description of Artificial Sequence: Synthetic oligonucleotide”
uaaaguacaauugugagaccagcc

SEQ ID NO: 601 - RNA, 24 nt, Artificial Sequence
  source: /note=“Description of Artificial Sequence: Synthetic oligonucleotide”
guaaaguacaauugugagaccagc

SEQ ID NO: 602 - RNA, 24 nt, Artificial Sequence
  source: /note=“Description of Artificial Sequence: Synthetic oligonucleotide”
guauuccuuuuacauaauacaaag

SEQ ID NO: 603 - RNA, 24 nt, Artificial Sequence
  source: /note=“Description of Artificial Sequence: Synthetic oligonucleotide”
guuguguauuccuuuuacauaaua

SEQ ID NO: 604 - RNA, 24 nt, Artificial Sequence
  source: /note=“Description of Artificial Sequence: Synthetic oligonucleotide”
auccugcauuguugccuguaagaa

SEQ ID NO: 605 - RNA, 24 nt, Artificial Sequence
  source: /note=“Description of Artificial Sequence: Synthetic oligonucleotide”
uuccaacuggggacgccucuguuc

SEQ ID NO: 606 - RNA, 24 nt, Artificial Sequence
  source: /note=“Description of Artificial Sequence: Synthetic oligonucleotide”
uuggaagaacucauuaccgcugcc

SEQ ID NO: 607 - RNA, 24 nt, Artificial Sequence
  source: /note=“Description of Artificial Sequence: Synthetic oligonucleotide”
ucauuaccgcugcccaaaauuuga

SEQ ID NO: 608 - RNA, 24 nt, Artificial Sequence
  source: /note=“Description of Artificial Sequence: Synthetic oligonucleotide”
cucuugauugcuggucuuguuuuu

SEQ ID NO: 609 - RNA, 24 nt, Artificial Sequence
  source: /note=“Description of Artificial Sequence: Synthetic oligonucleotide”
guuuuuuaacaagcaugggacaca

SEQ ID NO: 610 - RNA, 24 nt, Artificial Sequence
  source: /note=“Description of Artificial Sequence: Synthetic oligonucleotide”
cuuugugugucccaugcuuguuaa

SEQ ID NO: 611 - RNA, 24 nt, Artificial Sequence
  source: /note=“Description of Artificial Sequence: Synthetic oligonucleotide”
gcuuugugugucccaugcuuguua

SEQ ID NO: 612 - RNA, 24 nt, Artificial Sequence
  source: /note=“Description of Artificial Sequence: Synthetic oligonucleotide”
ugcuuugugugucccaugcuuguu

SEQ ID NO: 613 - RNA, 24 nt, Artificial Sequence
  source: /note=“Description of Artificial Sequence: Synthetic oligonucleotide”
uugcuuugugugucccaugcuugu

SEQ ID NO: 614 - RNA, 24 nt, Artificial Sequence
  source: /note=“Description of Artificial Sequence: Synthetic oligonucleotide”
agcaagaugcaugacaaguuucaa

SEQ ID NO: 615 - RNA, 24 nt, Artificial Sequence
  source: /note=“Description of Artificial Sequence: Synthetic oligonucleotide”
gcaagaugcaugacaaguuucaau

SEQ ID NO: 616 - RNA, 24 nt, Artificial Sequence
  source: /note=“Description of Artificial Sequence: Synthetic oligonucleotide”
caagaugcaugacaaguuucaaua

SEQ ID NO: 617 - RNA, 24 nt, Artificial Sequence
  source: /note=“Description of Artificial Sequence: Synthetic oligonucleotide”
gauauaugaacuuaaguuuuuauu

SEQ ID NO: 618 - RNA, 24 nt, Artificial Sequence
  source: /note=“Description of Artificial Sequence: Synthetic oligonucleotide”
auagaaauccaauaauauauucac

SEQ ID NO: 619 - RNA, 24 nt, Artificial Sequence
  source: /note=“Description of Artificial Sequence: Synthetic oligonucleotide”
auuaagauguucaugaauuaucuu

SEQ ID NO: 620 - RNA, 24 nt, Artificial Sequence
  source: /note=“Description of Artificial Sequence: Synthetic oligonucleotide”
uaaguaauguguaugcuuuucugu

SEQ ID NO: 621 - RNA, 24 nt, Artificial Sequence
  source: /note=“Description of Artificial Sequence: Synthetic oligonucleotide”
aucuucuaacuuccucuuuaacag

SEQ ID NO: 622 - RNA, 24 nt, Artificial Sequence
  source: /note=“Description of Artificial Sequence: Synthetic oligonucleotide”
gaucuucuaacuuccucuuuaaca

SEQ ID NO: 623 - RNA, 24 nt, Artificial Sequence
  source: /note=“Description of Artificial Sequence: Synthetic oligonucleotide”
aucugagcucugaguggaaggcgg

SEQ ID NO: 624 - RNA, 24 nt, Artificial Sequence
  source: /note=“Description of Artificial Sequence: Synthetic oligonucleotide”
accguuuacuucaagagcugaggg

SEQ ID NO: 625 - RNA, 24 nt, Artificial Sequence
  source: /note=“Description of Artificial Sequence: Synthetic oligonucleotide”
cugcuuugcccucagcucuugaag

SEQ ID NO: 626 - RNA, 24 nt, Artificial Sequence
  source: /note=“Description of Artificial Sequence: Synthetic oligonucleotide”
ucucuuuggcucuagcuauuuguu

SEQ ID NO: 627 - RNA, 24 nt, Artificial Sequence
  source: /note=“Description of Artificial Sequence: Synthetic oligonucleotide”
cucuuuggcucuagcuauuuguuc

SEQ ID NO: 628 - RNA, 24 nt, Artificial Sequence
  source: /note=“Description of Artificial Sequence: Synthetic oligonucleotide”
cacuuuugaacaaauagcuagagc

SEQ ID NO: 629 - RNA, 24 nt, Artificial Sequence
  source: /note=“Description of Artificial Sequence: Synthetic oligonucleotide”
ucacuucauaguugcacuuuugaa

SEQ ID NO: 630 - RNA, 24 nt, Artificial Sequence
  source: /note=“Description of Artificial Sequence: Synthetic oligonucleotide”
augaagugaugacugggugagaga

SEQ ID NO: 631 - RNA, 24 nt, Artificial Sequence
  source: /note=“Description of Artificial Sequence: Synthetic oligonucleotide”
ugaagugaugacugggugagagag

SEQ ID NO: 632 - RNA, 24 nt, Artificial Sequence
  source: /note=“Description of Artificial Sequence: Synthetic oligonucleotide”
aagagaaaaauauauauauauaua

SEQ ID NO: 633 - RNA, 24 nt, Artificial Sequence
  source: /note=“Description of Artificial Sequence: Synthetic oligonucleotide”
gaauuagcugucuauagaaagaga

SEQ ID NO: 634 - RNA, 24 nt, Artificial Sequence
  source: /note=“Description of Artificial Sequence: Synthetic oligonucleotide”
ugaauuagcugucuauagaaagag

SEQ ID NO: 635 - RNA, 24 nt, Artificial Sequence
  source: /note=“Description of Artificial Sequence: Synthetic oligonucleotide”
agcuaauucauuuuuuuacuguuu

SEQ ID NO: 636 - RNA, 24 nt, Artificial Sequence
  source: /note=“Description of Artificial Sequence: Synthetic oligonucleotide”
augaauuagcugucuauagaaaga

SEQ ID NO: 637 - RNA, 24 nt, Artificial Sequence
  source: /note=“Description of Artificial Sequence: Synthetic oligonucleotide”
gcuaauucauuuuuuuacuguuuu

SEQ ID NO: 638 - RNA, 24 nt, Artificial Sequence
  source: /note=“Description of Artificial Sequence: Synthetic oligonucleotide”
aaaaaaaugaauuagcugucuaua

SEQ ID NO: 639 - RNA, 24 nt, Artificial Sequence
  source: /note=“Description of Artificial Sequence: Synthetic oligonucleotide”
uuaaaauuuuuauauuacagaaua

SEQ ID NO: 640 - RNA, 24 nt, Artificial Sequence
  source: /note=“Description of Artificial Sequence: Synthetic oligonucleotide”
uaaaauuuuuauauuacagaauau

SEQ ID NO: 641 - RNA, 24 nt, Artificial Sequence
  source: /note=“Description of Artificial Sequence: Synthetic oligonucleotide”
auauaaaaauuuuaaaacaguaaa

SEQ ID NO: 642 - RNA, 24 nt, Artificial Sequence
  source: /note=“Description of Artificial Sequence: Synthetic oligonucleotide”
aauauaaaaauuuuaaaacaguaa

SEQ ID NO: 643 - RNA, 24 nt, Artificial Sequence
  source: /note=“Description of Artificial Sequence: Synthetic oligonucleotide”
uaauauaaaaauuuuaaaacagua

SEQ ID NO: 644 - RNA, 24 nt, Artificial Sequence
  source: /note=“Description of Artificial Sequence: Synthetic oligonucleotide”
guaauauaaaaauuuuaaaacagu

SEQ ID NO: 645 - RNA, 24 nt, Artificial Sequence
  source: /note=“Description of Artificial Sequence: Synthetic oligonucleotide”
uguaauauaaaaauuuuaaaacag

SEQ ID NO: 646 - RNA, 24 nt, Artificial Sequence
  source: /note=“Description of Artificial Sequence: Synthetic oligonucleotide”
uauauucuguaauauaaaaauuuu

SEQ ID NO: 647 - RNA, 24 nt, Artificial Sequence
  source: /note=“Description of Artificial Sequence: Synthetic oligonucleotide”
uuauauucuguaauauaaaaauuu

SEQ ID NO: 648 - RNA, 24 nt, Artificial Sequence
  source: /note=“Description of Artificial Sequence: Synthetic oligonucleotide”
cagaauauaaaagauagucuacaa

SEQ ID NO: 649 - RNA, 24 nt, Artificial Sequence
  source: /note=“Description of Artificial Sequence: Synthetic oligonucleotide”
cuaucuuuuauauucuguaauaua

SEQ ID NO: 650 - RNA, 24 nt, Artificial Sequence
  source: /note=“Description of Artificial Sequence: Synthetic oligonucleotide”
acuaucuuuuauauucuguaauau

SEQ ID NO: 651 - RNA, 24 nt, Artificial Sequence
  source: /note=“Description of Artificial Sequence: Synthetic oligonucleotide”
gacuaucuuuuauauucuguaaua

SEQ ID NO: 652 - RNA, 24 nt, Artificial Sequence
  source: /note=“Description of Artificial Sequence: Synthetic oligonucleotide”
cauagcaagaagacagcagcauug

SEQ ID NO: 653 - RNA, 24 nt, Artificial Sequence
  source: /note=“Description of Artificial Sequence: Synthetic oligonucleotide”
cauuuuguuaacuuuuucccauug

SEQ ID NO: 654 - RNA, 24 nt, Artificial Sequence
  source: /note=“Description of Artificial Sequence: Synthetic oligonucleotide”
cauauauuuuucuugauacuugca

SEQ ID NO: 655 - RNA, 24 nt, Artificial Sequence
  source: /note=“Description of Artificial Sequence: Synthetic oligonucleotide”
aaaucauuucugcaaguaucaaga

SEQ ID NO: 656 - RNA, 24 nt, Artificial Sequence
  source: /note=“Description of Artificial Sequence: Synthetic oligonucleotide”
caaaucauuucugcaaguaucaag

SEQ ID NO: 657 - RNA, 24 nt, Artificial Sequence
  source: /note=“Description of Artificial Sequence: Synthetic oligonucleotide”
acaaaucauuucugcaaguaucaa

SEQ ID NO: 658 - RNA, 24 nt, Artificial Sequence
  source: /note=“Description of Artificial Sequence: Synthetic oligonucleotide”
auaaauucuacaguucccugaaaa

SEQ ID NO: 659 - RNA, 24 nt, Artificial Sequence
  source: /note=“Description of Artificial Sequence: Synthetic oligonucleotide”
gaauuuauuucaguacccuccaug

SEQ ID NO: 660 - RNA, 24 nt, Artificial Sequence
  source: /note=“Description of Artificial Sequence: Synthetic oligonucleotide”
aauuuauuucaguacccuccaugg

SEQ ID NO: 661 - RNA, 24 nt, Artificial Sequence
  source: /note=“Description of Artificial Sequence: Synthetic oligonucleotide”
ugaaauaaauucuacaguucccug

SEQ ID NO: 662 - RNA, 24 nt, Artificial Sequence
  source: /note=“Description of Artificial Sequence: Synthetic oligonucleotide”
auuuauuucaguacccuccaugga

SEQ ID NO: 663 - RNA, 24 nt, Artificial Sequence
  source: /note=“Description of Artificial Sequence: Synthetic oligonucleotide”
cugaaauaaauucuacaguucccu

SEQ ID NO: 664 - RNA, 24 nt, Artificial Sequence
  source: /note=“Description of Artificial Sequence: Synthetic oligonucleotide”
uuuauuucaguacccuccauggaa

SEQ ID NO: 665 - RNA, 24 nt, Artificial Sequence
  source: /note=“Description of Artificial Sequence: Synthetic oligonucleotide”
uacccuccauggaaaaaagacagg

SEQ ID NO: 666 - RNA, 24 nt, Artificial Sequence
  source: /note=“Description of Artificial Sequence: Synthetic oligonucleotide”
acccuccauggaaaaaagacaggg

SEQ ID NO: 667 - RNA, 24 nt, Artificial Sequence
  source: /note=“Description of Artificial Sequence: Synthetic oligonucleotide”
cccuccauggaaaaaagacaggga

SEQ ID NO: 668 - RNA, 24 nt, Artificial Sequence
  source: /note=“Description of Artificial Sequence: Synthetic oligonucleotide”
uuuuuuccauggaggguacugaaa

SEQ ID NO: 669 - RNA, 24 nt, Artificial Sequence
  source: /note=“Description of Artificial Sequence: Synthetic oligonucleotide”
ugucuuuuuuccauggaggguacu

SEQ ID NO: 670 - RNA, 24 nt, Artificial Sequence
  source: /note=“Description of Artificial Sequence: Synthetic oligonucleotide”
ccuugagcaagaaccaugcaaacu

SEQ ID NO: 671 - RNA, 24 nt, Artificial Sequence
  source: /note=“Description of Artificial Sequence: Synthetic oligonucleotide”
ugcucaaggaaugcauuuucuuau

SEQ ID NO: 672 - RNA, 24 nt, Artificial Sequence
  source: /note=“Description of Artificial Sequence: Synthetic oligonucleotide”
gcucaaggaaugcauuuucuuaug

SEQ ID NO: 673 - RNA, 24 nt, Artificial Sequence
  source: /note=“Description of Artificial Sequence: Synthetic oligonucleotide”
ugcauuccuugagcaagaaccaug

SEQ ID NO: 674 - RNA, 24 nt, Artificial Sequence
  source: /note=“Description of Artificial Sequence: Synthetic oligonucleotide”
gaaaauuuauuuccacauguaggu

SEQ ID NO: 675 - RNA, 24 nt, Artificial Sequence
  source: /note=“Description of Artificial Sequence: Synthetic oligonucleotide”
aaaauuuauuuccacauguagguc

SEQ ID NO: 676 - RNA, 24 nt, Artificial Sequence
  source: /note=“Description of Artificial Sequence: Synthetic oligonucleotide”
aaauuuauuuccacauguagguca

SEQ ID NO: 677 - RNA, 24 nt, Artificial Sequence
  source: /note=“Description of Artificial Sequence: Synthetic oligonucleotide”
cauguggaaauaaauuuucauaag

SEQ ID NO: 678 - RNA, 24 nt, Artificial Sequence
  source: /note=“Description of Artificial Sequence: Synthetic oligonucleotide”
acauguggaaauaaauuuucauaa

SEQ ID NO: 679 - RNA, 24 nt, Artificial Sequence
  source: /note=“Description of Artificial Sequence: Synthetic oligonucleotide”
ccacauguaggucaaaaauguaau

SEQ ID NO: 680 - RNA, 24 nt, Artificial Sequence
  source: /note=“Description of Artificial Sequence: Synthetic oligonucleotide”
cacauguaggucaaaaauguaaug

SEQ ID NO: 681 - RNA, 24 nt, Artificial Sequence
  source: /note=“Description of Artificial Sequence: Synthetic oligonucleotide”
acauguaggucaaaaauguaauga

SEQ ID NO: 682 - RNA, 24 nt, Artificial Sequence
  source: /note=“Description of Artificial Sequence: Synthetic oligonucleotide”
acauuuuugaccuacauguggaaa

SEQ ID NO: 683 - RNA, 24 nt, Artificial Sequence
  source: /note=“Description of Artificial Sequence: Synthetic oligonucleotide”
cauuacauuuuugaccuacaugug

SEQ ID NO: 684 - RNA, 24 nt, Artificial Sequence
  source: /note=“Description of Artificial Sequence: Synthetic oligonucleotide”
aaaaauaucauggcuggauugcaa

SEQ ID NO: 685 - RNA, 24 nt, Artificial Sequence
  source: /note=“Description of Artificial Sequence: Synthetic oligonucleotide”
gcuggauugcaacaaaccaacagu

SEQ ID NO: 686 - RNA, 24 nt, Artificial Sequence
  source: /note=“Description of Artificial Sequence: Synthetic oligonucleotide”
cuggauugcaacaaaccaacagug

SEQ ID NO: 687 - RNA, 24 nt, Artificial Sequence
  source: /note=“Description of Artificial Sequence: Synthetic oligonucleotide”
ccuaugacuauggaugagagcauu

SEQ ID NO: 688 - RNA, 24 nt, Artificial Sequence
  source: /note=“Description of Artificial Sequence: Synthetic oligonucleotide”
uagguaagaagauuacugagacau

SEQ ID NO: 689 - RNA, 24 nt, Artificial Sequence
  source: /note=“Description of Artificial Sequence: Synthetic oligonucleotide”
auuacugagacauuaaauaacuug

SEQ ID NO: 690 - RNA, 24 nt, Artificial Sequence
  source: /note=“Description of Artificial Sequence: Synthetic oligonucleotide”
uuacugagacauuaaauaacuugu

SEQ ID NO: 691 - RNA, 24 nt, Artificial Sequence
  source: /note=“Description of Artificial Sequence: Synthetic oligonucleotide”
ggggaaaaauaugucaucagaguc

SEQ ID NO: 692 - RNA, 24 nt, Artificial Sequence
  source: /note=“Description of Artificial Sequence: Synthetic oligonucleotide”
caugaucuggaaccauacugggga

SEQ ID NO: 693 - RNA, 24 nt, Artificial Sequence
  source: /note=“Description of Artificial Sequence: Synthetic oligonucleotide”
acaugaucuggaaccauacugggg

SEQ ID NO: 694 - RNA, 24 nt, Artificial Sequence
  source: /note=“Description of Artificial Sequence: Synthetic oligonucleotide”
gacaugaucuggaaccauacuggg

SEQ ID NO: 695 - RNA, 24 nt, Artificial Sequence
  source: /note=“Description of Artificial Sequence: Synthetic oligonucleotide”
uacacacauacacaaagacaaaua

SEQ ID NO: 696 - RNA, 24 nt, Artificial Sequence
  source: /note=“Description of Artificial Sequence: Synthetic oligonucleotide”
uacacauacacacauacacaaaga

SEQ ID NO: 697 - RNA, 24 nt, Artificial Sequence
  source: /note=“Description of Artificial Sequence: Synthetic oligonucleotide”
aacacauacacauacacacauaca

SEQ ID NO: 698 - RNA, 24 nt, Artificial Sequence
  source: /note=“Description of Artificial Sequence: Synthetic oligonucleotide”
auuccagucaaauaggucuggccu

SEQ ID NO: 699 - RNA, 24 nt, Artificial Sequence
  source: /note=“Description of Artificial Sequence: Synthetic oligonucleotide”
uauuccagucaaauaggucuggcc

SEQ ID NO: 700 - RNA, 24 nt, Artificial Sequence
  source: /note=“Description of Artificial Sequence: Synthetic oligonucleotide”
gcuggcaaaccacacuauuccagu

SEQ ID NO: 701 - RNA, 24 nt, Artificial Sequence
  source: /note=“Description of Artificial Sequence: Synthetic oligonucleotide”
agucguuguguggcugacugcugg

SEQ ID NO: 702 - RNA, 24 nt, Artificial Sequence
  source: /note=“Description of Artificial Sequence: Synthetic oligonucleotide”
cgccagauaucaauuaggcauaga

SEQ ID NO: 703 - RNA, 24 nt, Artificial Sequence
  source: /note=“Description of Artificial Sequence: Synthetic oligonucleotide”
aaacuacucgauccugaagguugg

SEQ ID NO: 704 - RNA, 24 nt, Artificial Sequence
  source: /note=“Description of Artificial Sequence: Synthetic oligonucleotide”
cauacuaaaagcagugguagucca

SEQ ID NO: 705 - RNA, 24 nt, Artificial Sequence
  source: /note=“Description of Artificial Sequence: Synthetic oligonucleotide”
gaaaacauuaaacucuaccauacu

SEQ ID NO: 706 - RNA, 24 nt, Artificial Sequence
  source: /note=“Description of Artificial Sequence: Synthetic oligonucleotide”
ugaaaacauuaaacucuaccauac

SEQ ID NO: 707 - RNA, 24 nt, Artificial Sequence
  source: /note=“Description of Artificial Sequence: Synthetic oligonucleotide”
uuguucauuauccuuuuagagucu

SEQ ID NO: 708 - RNA, 24 nt, Artificial Sequence
  source: /note=“Description of Artificial Sequence: Synthetic oligonucleotide”
aaaggauaaugaacaaaucaaagu

SEQ ID NO: 709 - RNA, 24 nt, Artificial Sequence
  source: /note=“Description of Artificial Sequence: Synthetic oligonucleotide”
uauccuuuuagagucucaaauaua

SEQ ID NO: 710 - RNA, 24 nt, Artificial Sequence
  source: /note=“Description of Artificial Sequence: Synthetic oligonucleotide”
acucuaaaaggauaaugaacaaau

SEQ ID NO: 711 - RNA, 24 nt, Artificial Sequence
  source: /note=“Description of Artificial Sequence: Synthetic oligonucleotide”
uuuuagagucucaaauauagaaac

SEQ ID NO: 712 - RNA, 24 nt, Artificial Sequence
  source: /note=“Description of Artificial Sequence: Synthetic oligonucleotide”
uuuagagucucaaauauagaaacc

SEQ ID NO: 713 - RNA, 24 nt, Artificial Sequence
  source: /note=“Description of Artificial Sequence: Synthetic oligonucleotide”
uuagagucucaaauauagaaacca

SEQ ID NO: 714 - RNA, 24 nt, Artificial Sequence
  source: /note=“Description of Artificial Sequence: Synthetic oligonucleotide”
uugagacucuaaaaggauaaugaa

SEQ ID NO: 715 - RNA, 24 nt, Artificial Sequence
  source: /note=“Description of Artificial Sequence: Synthetic oligonucleotide”
uuugguuucuauauuugagacucu

SEQ ID NO: 716 - RNA, 24 nt, Artificial Sequence
  source: /note=“Description of Artificial Sequence: Synthetic oligonucleotide”
uuuugguuucuauauuugagacuc

SEQ ID NO: 717 - RNA, 24 nt, Artificial Sequence
  source: /note=“Description of Artificial Sequence: Synthetic oligonucleotide”
agcauugaagccauccaggaagug

SEQ ID NO: 718 - RNA, 24 nt, Artificial Sequence
  source: /note=“Description of Artificial Sequence: Synthetic oligonucleotide”
gcuucaaugcucacuuguugaggc

SEQ ID NO: 719 - RNA, 24 nt, Artificial Sequence
  source: /note=“Description of Artificial Sequence: Synthetic oligonucleotide”
ggcuucaaugcucacuuguugagg

SEQ ID NO: 720 - RNA, 24 nt, Artificial Sequence
  source: /note=“Description of Artificial Sequence: Synthetic oligonucleotide”
aguggaaauguugccaaggccacc

SEQ ID NO: 721 - RNA, 24 nt, Artificial Sequence
  source: /note=“Description of Artificial Sequence: Synthetic oligonucleotide”
guugccaaggccaccuaaagugac

SEQ ID NO: 722 - RNA, 24 nt, Artificial Sequence
  source: /note=“Description of Artificial Sequence: Synthetic oligonucleotide”
gaagaacauuuucaguuacaucau

SEQ ID NO: 723 - RNA, 24 nt, Artificial Sequence
  source: /note=“Description of Artificial Sequence: Synthetic oligonucleotide”
aucaaaugcacuauucucaacagg

SEQ ID NO: 724 - RNA, 24 nt, Artificial Sequence
  source: /note=“Description of Artificial Sequence: Synthetic oligonucleotide”
auagugcauuugaugauguaacug

SEQ ID NO: 725 - RNA, 24 nt, Artificial Sequence
  source: /note=“Description of Artificial Sequence: Synthetic oligonucleotide”
aauagugcauuugaugauguaacu

SEQ ID NO: 726 - RNA, 24 nt, Artificial Sequence
  source: /note=“Description of Artificial Sequence: Synthetic oligonucleotide”
acuauucucaacagguaaagugug

SEQ ID NO: 727 - RNA, 24 nt, Artificial Sequence
  source: /note=“Description of Artificial Sequence: Synthetic oligonucleotide”
uaccuaaaaaugcauauaaaacag

SEQ ID NO: 728 - RNA, 24 nt, Artificial Sequence
  source: /note=“Description of Artificial Sequence: Synthetic oligonucleotide”
auaccuaaaaaugcauauaaaaca

SEQ ID NO: 729 - RNA, 24 nt, Artificial Sequence
  source: /note=“Description of Artificial Sequence: Synthetic oligonucleotide”
cacguaauaccuaaaaaugcauau

SEQ ID NO: 730 - RNA, 24 nt, Artificial Sequence
  source: /note=“Description of Artificial Sequence: Synthetic oligonucleotide”
gcacguaauaccuaaaaaugcaua

SEQ ID NO: 731 - RNA, 24 nt, Artificial Sequence
  source: /note=“Description of Artificial Sequence: Synthetic oligonucleotide”
auauauaugugcacguaauaccua

SEQ ID NO: 732 - RNA, 24 nt, Artificial Sequence
  source: /note=“Description of Artificial Sequence: Synthetic oligonucleotide”
uauauauaugugcacguaauaccu

SEQ ID NO: 733 - RNA, 24 nt, Artificial Sequence
  source: /note=“Description of Artificial Sequence: Synthetic oligonucleotide”
auauauauaugugcacguaauacc

SEQ ID NO: 734 - RNA, 24 nt, Artificial Sequence
  source: /note=“Description of Artificial Sequence: Synthetic oligonucleotide”
cugcacaauauuauaguuguugcu

SEQ ID NO: 735 - RNA, 24 nt, Artificial Sequence
  source: /note=“Description of Artificial Sequence: Synthetic oligonucleotide”
auaaaaagagaaagauggaggaac

SEQ ID NO: 736 - RNA, 24 nt, Artificial Sequence
  source: /note=“Description of Artificial Sequence: Synthetic oligonucleotide”
caccuagugaacuccauaaaaaga

SEQ ID NO: 737 - RNA, 24 nt, Artificial Sequence
  source: /note=“Description of Artificial Sequence: Synthetic oligonucleotide”
auggugcaccuagugaacuccaua

SEQ ID NO: 738 - RNA, 24 nt, Artificial Sequence
  source: /note=“Description of Artificial Sequence: Synthetic oligonucleotide”
aauggugcaccuagugaacuccau

SEQ ID NO: 739 - RNA, 24 nt, Artificial Sequence
  source: /note=“Description of Artificial Sequence: Synthetic oligonucleotide”
gaauggugcaccuagugaacucca

SEQ ID NO: 740 - RNA, 24 nt, Artificial Sequence
  source: /note=“Description of Artificial Sequence: Synthetic oligonucleotide”
gaccaaauguucagaugcaauuau

SEQ ID NO: 741 - RNA, 24 nt, Artificial Sequence
  source: /note=“Description of Artificial Sequence: Synthetic oligonucleotide”
ucgcucacucacccugcaaaggac

SEQ ID NO: 742 - RNA, 24 nt, Artificial Sequence
  source: /note=“Description of Artificial Sequence: Synthetic oligonucleotide”
agugagcgagaggcugcuuuggaa

SEQ ID NO: 743 - RNA, 24 nt, Artificial Sequence
  source: /note=“Description of Artificial Sequence: Synthetic oligonucleotide”
gcagccucucgcucacucacccug

SEQ ID NO: 744 - RNA, 24 nt, Artificial Sequence
  source: /note=“Description of Artificial Sequence: Synthetic oligonucleotide”
uugcaguaaucuaugaguuucuuc

SEQ ID NO: 745 - RNA, 24 nt, Artificial Sequence
  source: /note=“Description of Artificial Sequence: Synthetic oligonucleotide”
cugcaacaguucccccuggaccug

SEQ ID NO: 746 - RNA, 24 nt, Artificial Sequence
  source: /note=“Description of Artificial Sequence: Synthetic oligonucleotide”
ugcaacaguucccccuggaccugg

SEQ ID NO: 747 - RNA, 24 nt, Artificial Sequence
  source: /note=“Description of Artificial Sequence: Synthetic oligonucleotide”
uuucuugccuggcuuacagaagcu

SEQ ID NO: 748 - RNA, 24 nt, Artificial Sequence
  source: /note=“Description of Artificial Sequence: Synthetic oligonucleotide”
uuucagcuucuguaagccaggcaa

SEQ ID NO: 749 - RNA, 24 nt, Artificial Sequence
  source: /note=“Description of Artificial Sequence: Synthetic oligonucleotide”
guccuacaggaugcuacccguaag

SEQ ID NO: 750 - RNA, 24 nt, Artificial Sequence
  source: /note=“Description of Artificial Sequence: Synthetic oligonucleotide”
ggcuccuagaagacuccaagggag

SEQ ID NO: 751 - RNA, 24 nt, Artificial Sequence
  source: /note=“Description of Artificial Sequence: Synthetic oligonucleotide”
gcuccuagaagacuccaagggagu

SEQ ID NO: 752 - RNA, 24 nt, Artificial Sequence
  source: /note=“Description of Artificial Sequence: Synthetic oligonucleotide”
cuccaagggaguaaaagagcugau

SEQ ID NO: 753 - RNA, 24 nt, Artificial Sequence
  source: /note=“Description of Artificial Sequence: Synthetic oligonucleotide”
uggauccacaagagugcuaaagcg

SEQ ID NO: 754 - RNA, 24 nt, Artificial Sequence
  source: /note=“Description of Artificial Sequence: Synthetic oligonucleotide”
guucaauuggauccacaagagugc

SEQ ID NO: 755 - RNA, 24 nt, Artificial Sequence
  source: /note=“Description of Artificial Sequence: Synthetic oligonucleotide”
uacuuguaacugacaagccaggga

SEQ ID NO: 756 - RNA, 24 nt, Artificial Sequence
  source: /note=“Description of Artificial Sequence: Synthetic oligonucleotide”
acuuguaacugacaagccagggac

SEQ ID NO: 757 - RNA, 24 nt, Artificial Sequence
  source: /note=“Description of Artificial Sequence: Synthetic oligonucleotide”
guaacugacaagccagggacaaaa

SEQ ID NO: 758 - RNA, 24 nt, Artificial Sequence
  source: /note=“Description of Artificial Sequence: Synthetic oligonucleotide”
uaacugacaagccagggacaaaac

SEQ ID NO: 759 - RNA, 24 nt, Artificial Sequence
  source: /note=“Description of Artificial Sequence: Synthetic oligonucleotide”
ucccuggcuugucaguuacaagua

SEQ ID NO: 760 - RNA, 24 nt, Artificial Sequence
  source: /note=“Description of Artificial Sequence: Synthetic oligonucleotide”
cagaguaacagucugaguaggagc

SEQ ID NO: 761 - RNA, 24 nt, Artificial Sequence
  source: /note=“Description of Artificial Sequence: Synthetic oligonucleotide”
uacuuuguuuagcaauacauggua

SEQ ID NO: 762 - RNA, 24 nt, Artificial Sequence
  source: /note=“Description of Artificial Sequence: Synthetic oligonucleotide”
uggcucaaauuguuacucuucaau

SEQ ID NO: 763 - RNA, 24 nt, Artificial Sequence
  source: /note=“Description of Artificial Sequence: Synthetic oligonucleotide”
cuuucaaagaacuuugcagagccu

SEQ ID NO: 764 - RNA, 24 nt, Artificial Sequence
  source: /note=“Description of Artificial Sequence: Synthetic oligonucleotide”
guugaagccauuuuguugcucuuu

SEQ ID NO: 765 - RNA, 24 nt, Artificial Sequence
  source: /note=“Description of Artificial Sequence: Synthetic oligonucleotide”
guugaagccauuuuauugcucuuu

SEQ ID NO: 766 - RNA, 24 nt, Artificial Sequence
  source: /note=“Description of Artificial Sequence: Synthetic oligonucleotide”
uuuugaggcucugcaaaguucuuu

SEQ ID NO: 767 - RNA, 24 nt, Artificial Sequence
  source: /note=“Description of Artificial Sequence: Synthetic oligonucleotide”
aguuauuaaugcauagauauucag

SEQ ID NO: 768 - RNA, 24 nt, Artificial Sequence
  source: /note=“Description of Artificial Sequence: Synthetic oligonucleotide”
uauaauaugcccuguaauauaaua

SEQ ID NO: 769 - RNA, 24 nt, Artificial Sequence
  source: /note=“Description of Artificial Sequence: Synthetic oligonucleotide”
uaaaggccaaaccucggcuuaccu

SEQ ID NO: 770 - RNA, 24 nt, Artificial Sequence
  source: /note=“Description of Artificial Sequence: Synthetic oligonucleotide”
ucaauaucuuugaaggacucuggg

SEQ ID NO: 771 - DNA, 25 bp, Artificial Sequence
  source: /note=“Description of Artificial Sequence: Synthetic primer”
caccgcactagagtaacagtctgac

SEQ ID NO: 772 - DNA, 24 bp, Artificial Sequence
  source: /note=“Description of Artificial Sequence: Synthetic primer”
aaacccagtcagactgttactctc

SEQ ID NO: 773 - DNA, 59 bp, Artificial Sequence
  source: /note=“Description of Artificial Sequence: Synthetic primer”
tcgtcggcagcgtcagatgtgtataagagacaggaaattttacctcaaactgttgcttc

SEQ ID NO: 774 - DNA, 60 bp, Artificial Sequence
  source: /note=“Description of Artificial Sequence: Synthetic primer”
gtctcgtgggctcggagatgtgtataagagacaggagggaaatggaaagtgacaatatac

SEQ ID NO: 775 - DNA, 43 bp, Artificial Sequence
  source: /note=“Description of Artificial Sequence: Synthetic primer”
aatgatacggcgaccaccgagatctacactcgtcggcagcgtc

SEQ ID NO: 776 - DNA, 45 bp, Artificial Sequence
  source: /note=“Description of Artificial Sequence: Synthetic primer”
caagcagaagacggcatacgagatacatcggtctcgtgggctcgg

SEQ ID NO: 777 - DNA, 45 bp, Artificial Sequence
  source: /note=“Description of Artificial Sequence: Synthetic primer”
caagcagaagacggcatacgagattggtcagtctcgtgggctcgg

SEQ ID NO: 778 - DNA, 45 bp, Artificial Sequence
  source: /note=“Description of Artificial Sequence: Synthetic primer”
caagcagaagacggcatacgagatcactgtgtctcgtgggctcgg

SEQ ID NO: 779 - DNA, 45 bp, Artificial Sequence
  source: /note=“Description of Artificial Sequence: Synthetic primer”
caagcagaagacggcatacgagatattggcgtctcgtgggctcgg

SEQ ID NO: 780 - DNA, 45 bp, Artificial Sequence
  source: /note=“Description of Artificial Sequence: Synthetic primer”
caagcagaagacggcatacgagatgatctggtctcgtgggctcgg

SEQ ID NO: 781 - DNA, 45 bp, Artificial Sequence
  source: /note=“Description of Artificial Sequence: Synthetic primer”
caagcagaagacggcatacgagattacaaggtctcgtgggctcgg

SEQ ID NO: 782 - DNA, 45 bp, Artificial Sequence
  source: /note=“Description of Artificial Sequence: Synthetic primer”
caagcagaagacggcatacgagatcgtgatgtctcgtgggctcgg

SEQ ID NO: 783 - DNA, 45 bp, Artificial Sequence
  source: /note=“Description of Artificial Sequence: Synthetic primer”
caagcagaagacggcatacgagatgcctaagtctcgtgggctcgg

SEQ ID NO: 784 - DNA, 45 bp, Artificial Sequence
  source: /note=“Description of Artificial Sequence: Synthetic primer”
caagcagaagacggcatacgagattcaagtgtctcgtgggctcgg

SEQ ID NO: 785 - DNA, 45 bp, Artificial Sequence
  source: /note=“Description of Artificial Sequence: Synthetic primer”
caagcagaagacggcatacgagatagctaggtctcgtgggctcgg

SEQ ID NO: 786 - protein, 2158 aa, Mus musculus
MWWVDCYRDVKKTTKWNASKGKHDNSDDGKRDGTGKKKGSTRVHANNVNKARVKNNVDVNGSTDVDGNHKTGWNHWVKNVMKTMAGTNSKSWVRSTRNYVNVNTSSWSDGANAHSHRDDWNSVVSHSATRHANAKCGKDDVATTYDKKSMYTSVVSAVMRTSSKVTRHHHMHYSTVSAGYTSSSKRKSYATAAYVATSDSTSYSHARDKSDSSMTVNDSYTAVSWSADTRAGSNDVVKHAHGMMDTSHGVGNVGSVGKGKSDAVMNNSRWCRVASMKSKHKVMDNKKDDWTKTRTKKMGDDKCVHKVDVRVNSTHMVVVVDSSGDHATAAKVGDRWANCRWTDRWVDKWHTCSTWSKDAMKNTSGKDNMMSSHKSTKDKKKTMKSSNDSAKNKSVTKMWMNARWDNTKKSSASAVTTTSTTTVMTVTMVTTRMVKHAKKRTVDSRKRDVDTHSWTRSAVSSAVYRKGNSDKVNAARKAKRKDASRSAAVMANGVNASRASNSRWTCSRVNWYTNTYNMTTTANKTSTTSTAKSKCKDVNRSAKSKKGGMDADVATNHNHDGVRAKKTDTMRYTMSSRTWSSKSVYSVTYMRGKASSKNGNYSDTVKMAKKASCKYSGHWKKSSVSCKHMNKRKNHKTKWMAVDVKWAGDAKKKCRVGDTSNSVNGGKKSAASRTRNTWDHCRVYTRKAKAGDKTVSKDSMHWMTAYRDYKTDTAVMKRAKAKTKVKTTVNSVAHASAAKKTTTNYWCTRNGKCKTVWACWHSYKANKWNVKKTMNVAGTVSNMHHSNNRATTDGGVMDNTNSRWRHAVRKKSSAKSHSDKAAYTDKVDAAMAKSDTSHSMKKHNGKDANRVSDVAKKDVSMKRKANRSKMDVKMHATKSVVSSHCVNYKSSVKSVMVKTGRVKKTNKDRVTAKHYNGAKVTRKKCKSRKMRKMNVTWAATDTTKRSAVGMSNDSVAWGKATKKKAHKSVTGSKMVGKKTVDKSNSNWAVTSRVWNYKHMTDNTKWHADDSKKKKDKRKAMNDMRKVDSTRDAAKMANRGDHCRKVVSNRRAASHRKTGKASKNSDKAGVNKDNKDMSDNGTVNRGDNRTDRKRKKTKHNAKDRSRRKKASHWYYKRADDKCDKKASRDRKKDRKKKNAVRRAGSNGAAMAVTSKRWRSNARRNAHTHTMVVTTDMDVSYVSTYTSHASVDHNTCAKDDKSKNKDNSGRDHKKKTAASATSMKVKVAVAMDGKHRMYKRGRDRSVKWRHHYDMKVNWNVKKTNNWHAKYKWYKDGGRAVVRTNATGSSKTDVNKGSSRWHDCKARRKRKNVSRDNVWADNATGDKVKARGKNTGGAVVSARDKKKKTNWKVSRAKGVHKDRDHWSRNYNSAGDKVTVHGKADVRSKGHYKKSTVKRKDRSWAVNHRRTKDRAGSTTGASASTVTVTSVVTKTVSKMSSVAADNRAWTTDWSDRVKSRVMVGDDNMKKATDRRTAANKNKTSNARTTDRRWDVNRRNMKDSTWAKAVGVRGKDSWKGHTVDAKKTTKAKDRRSVDVANDAKRDYSADDTRKVHMTNNTSWGNHKRVSAATHRDKSWTATTANVDASRKKDSRGVRMKWDGTHTDYHNDNGKRSGSDARRDNMNKWSKKSNRSHASSDWKRHSVWKDDSRAGGDAVKNDHRAKRKTKVMSTTVRTGKYRRANVTRRKAVNAWDKNRSADWRKDARAADDKRAVKGSWVGDDSDHKVKARGAKNVNRVNDAHTTGSYNSTDNTRWRVAVDRVRHAHRDGASHSTSVGWRASNKVYYNHTTTCWDHKMTYSADNNVRSAYRTAMKRRKACDSSAACDADHNKNDMDNCTTYDRHNNVNVCVDMCNWNVYDTGRTGRRVSKTGSCKAHDKYRYKVASSTGCDRRGHDSRGVASGGSNSVRSCANNKAADWMRSMVWVHRVAAATAKHAKCNCKCGRYRSKHNYDCSCSGRVAKGHKMHYMVYCTTTSGDVRDAKVKNKRTKRYAKHRMGYVTVGDNMTVTNWVDSAASSSHDDTHSRHYASRAMNSNGSYNDSSNSDDHHYCSNDSSRSASSRGRADNRNAYDRKHHKGSSMMTSSRDAAAKRHKGRARMDHNKSHRRAAKVNGTTVSSSTSRSDSSMRVVGSTSSMGDSDTSTGVMNNSSSRGRNAGKMRDTM

SEQ ID NO: 787 - DNA, 450 bp, Unknown
  source: /note=“Description of Unknown: CK8 promoter sequence”
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 788 - DNA, 436 bp, Unknown
  source: /note=“Description of Unknown: CK8e promoter sequence”
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc